(19) United States
(12) Patent Application Publication
(10) Pub. No.: US 2026/0234116 A1
(43) Pub. Date: Aug. 13, 2026

(54) CYTOTOXIC EPOXYKETONE COMPOUNDS, PRECURSORS, COMPOSITIONS, AND METHODS OF USE

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Derek S. Tan, New York, NY (US); Broderick Cameron Corless, New York, NY (US); Raphael Geiben, New York, NY (US)

(21) Appl. No.: 19/474,999

(22) PCT Filed: Apr. 10, 2024
(86) PCT No.: PCT/US2024/023859

§ 371 Date: Oct. 13, 2025

Related U.S. Application Data
(60) Provisional application No. 63/459,444, filed on Apr. 14, 2023.

(51) Int. Cl. C07D 303/36; A61K 31/164; A61K 31/336; C07C 237/22

(52) U.S. Cl. CPC C07D 303/36; A61K 31/164; A61K 31/336; C07C 237/22

(57) ABSTRACT

The present disclosure relates to epoxyketone compounds according to Formula (I) or a pharmaceutically acceptable salt and/or solvate thereof, P-ketoacid precursors of such epoxyketone compounds, as well as compositions and uses thereof.

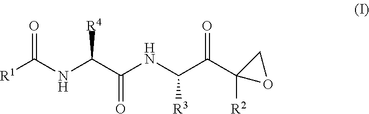

### CROSS-REFERENCE TO RELATED APPLICATIONS

This application claims the benefit of and priority to U.S. Provisional Appl. No. 63/459,444, filed Apr. 14, 2023, the contents of which are incorporated herein by reference in their entirety for any and all purposes.

### U.S. GOVERNMENT SUPPORT

This invention was made with government support under P01 CA023766 and P30 CA008748 awarded by the National Institutes of Health. The government has certain rights in the invention.

### FIELD

The present technology is directed to epoxyketone compounds as well as β-ketoacid precursors of such epoxyketone compounds. The present technology is also directed to compositions including such epoxyketone compounds, compositions including such β-ketoacid precursors, and respective methods of use.

### SUMMARY

In an aspect, the present technology provides an epoxyketone compound of Formula (I):

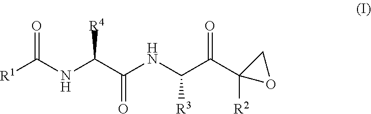

or a pharmaceutically acceptable salt and/or solvate thereof, wherein
   R¹ is alkyl, unsubstituted phenyl, or substituted phenyl;
   R² is H or C₁₋₆ alkyl optionally substituted with OH;
   R³ is C₁₋₆ alkyl optionally substituted with C₂₋₆ alkenyl or phenyl;
   R⁴ is C₁₋₆ alkyl optionally substituted with OH or phenyl; and
optionally wherein the epoxyketone compound is not one or more of

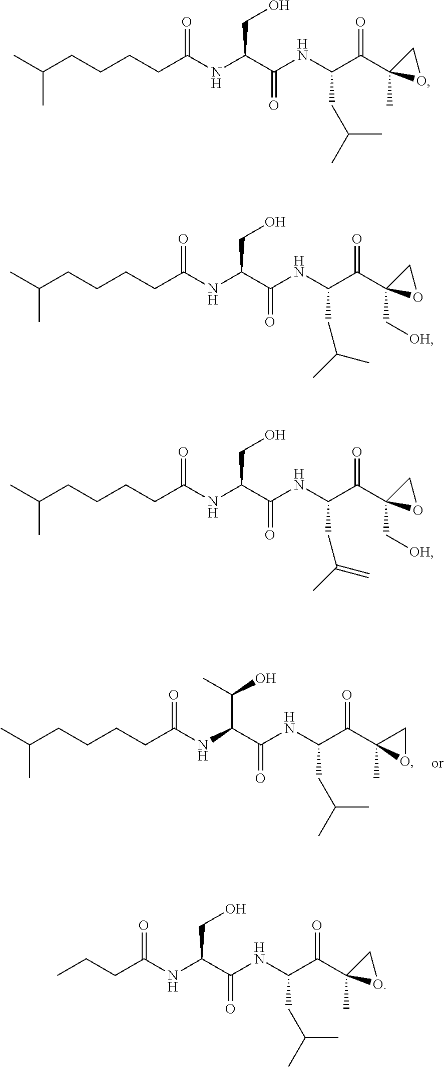 [SMILES: CC(C)CCCCC(=O)N[C@@H](CO)C(=O)N[C@@H](CC(C)C)C(=O)[C@@]1(C)CO1 ; CC(C)CCCCC(=O)N[C@@H](CO)C(=O)N[C@@H](CC(C)C)C(=O)[C@@]1(CO)CO1 ; C=C(C)C[C@H](NC(=O)[C@H](CO)NC(=O)CCCCC(C)C)C(=O)[C@@]1(CO)CO1 ; CC(C)CCCCC(=O)N[C@H](C(=O)N[C@@H](CC(C)C)C(=O)[C@@]1(C)CO1)[C@@H](C)O ; CCCC(=O)N[C@@H](CO)C(=O)N[C@@H](CC(C)C)C(=O)[C@@]1(C)CO1]

In another aspect, the present disclosure also provides a β-ketoacid useful in generating an epoxyketone compound of any embodiment described herein, the β-ketoacid being of Formula (A)

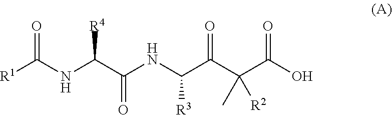

or a stereoisomer or a tautomer thereof, or a pharmaceutically acceptable salt and/or solvate thereof, wherein
   R¹ is alkyl, unsubstituted phenyl, or substituted phenyl;
   R² is H or C₁₋₆ alkyl optionally substituted with OH;
   R³ is C₁₋₆ alkyl optionally substituted with C₂₋₆ alkenyl or phenyl;
   R⁴ is C₁₋₆ alkyl optionally substituted with OH or phenyl.

In an aspect, a composition is provided that includes an epoxyketone compound of any embodiment disclosed herein or a β-ketoacid of any embodiment disclosed herein (collectively, “a compound of the present technology” or the like), as well as a pharmaceutically acceptable carrier or one or more excipients, fillers or agents (collectively referred to hereafter as “pharmaceutically acceptable carrier” unless otherwise indicated and/or specified).

In a related aspect, a medicament for treating a disease associated with proteasome activity such as cancer (e.g., hematopoietic cancer, acute lymphoblastic leukemia (ALL), or multiple myeloma) in a subject is provided that includes a compound of any embodiment disclosed herein and optionally a pharmaceutically acceptable carrier.

In a related aspect, a pharmaceutical composition is provided that includes (i) an effective amount of a compound of any embodiment disclosed herein, wherein the effective amount of the compound is effective to treat a disease associated with proteasome activity such as cancer (e.g., hematopoietic cancer, acute lymphoblastic leukemia (ALL), or multiple myeloma); and (ii) a pharmaceutically acceptable carrier.

In further related aspects, the present technology provides methods including a compound of any aspect or embodiment disclosed herein and/or a composition of any embodiment disclosed herein and/or a medicament of any embodiment disclosed herein. Such methods include a method of treating a subject suffering from a disease associated with proteasome activity such as cancer (e.g., hematopoietic cancer, acute lymphoblastic leukemia (ALL), or multiple myeloma), where the method includes administering to the subject an effective amount of a compound of any embodiment disclosed herein.

In another aspect, the present disclosure also provides a process for preparing an epoxyketone compound of any embodiment disclosed herein, the process comprising contacting a β-ketoacid of any embodiment disclosed herein with an epoxyketone synthase to provide the epoxyketone compound.

### BRIEF DESCRIPTION OF THE DRAWINGS

FIG. 1 relates to biochemical evaluation of β-ketoacids 11a-d of the present technology as native substrate candidates for flavin-dependent decarboxylase-dehydrogenase-monooxygenase (“EpnF”), according to the working examples. In particular, FIG. 1 provides the results of EpnF-catalyzed formation of epoxyketones from β-ketoacids 11a-d as determined using the semiquantitative epoxide capture assay, where the EpnF substrate 11n was used as a positive control (pos), vehicle was used as a negative control (neg), the individual diastereomers 11c/c′ and 11d/d′ were tested separately (relative stereochemistry not determined), experiments were performed with 1 μM EpnF and 250 μM β-ketoacid substrate, and data represent mean±s.e.m. of n=3 biological replicates (each with 3 technical replicates).

FIG. 2 relates to biochemical evaluation of β-ketoacid substrate analogues 11e-r as substrates for EpnF, according to the working examples. EpnF-catalyzed formation of epoxyketones from β-ketoacids 11e-r as determined using the semiquantitative epoxide capture assay (β-ketoacid 11n was omitted). The epoxyketone product 12n (without EpnF) was used as a positive control (epoxide) and vehicle was used as a negative control (neg). Experiments were performed with 1 μM EpnF and 240 μM β-ketoacid substrate. Data represent mean±s.e.m. of n=2 biological replicates (each with 3 technical replicates).

FIG. 3 illustrates the results of EL4 cell viability after incubation with epoxyketone 12a, b-ketoacid 11a, or isopropyl ketone decarboxylation product 22a at various concentrations, according to the working examples. Neither the β-ketoacid substrate nor the isopropyl ketone byproduct exhibited cytotoxicity at up to 50 mM concentration (CellTitre Glo assay, 48 h).

FIG. 4 illustrates the results of EL4 cell viability after incubation with EpnF at various concentrations, according to the working examples. The enzyme exhibited no cytotoxicity at up to 3 μM concentration (CellTitre-Glo assay, 48 h).

FIGS. 5A-5B illustrate anticancer activity of the epoxyketones of the present technology, according to the working examples. FIG. 5A provides the results of proteasome inhibition by isolated epoxyketones in Jurkat cell lysates (abcam proteasome activity assay). Known inhibitors MG-132 and epoxomicin were used as positive controls. Fluorescence intensity normalized to DMSO negative control (neg). Data represent mean±s.e.m. of n=3 biological replicates (each with 2 technical replicates). Statistical significance relative to DMSO control assessed by one-way ANOVA with Dunnett's comparison test: ***p≤0.001, **p≤0.01, ns=not significant. FIG. 5B illustrates the cytotoxicity of β-ketoacid 11a activated in situ by recombinant, purified EpnF (0.5 μM), compared to isolated epoxyketone product 12a, against U266 multiple myeloma cells (CellTiter-Glo assay, 48 h). Data represent mean f standard deviation of n=3 biological replicates.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C, A and B, A and C, B and C, or the combination of A, B, and C.”

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; and nitriles (i.e., CN).

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4- 2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂ among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups may be substituted or unsubstituted. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, and —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. Heterocyclyl groups may be substituted or unsubstituted. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. The phrase includes heterocyclyl groups that have other groups, such as alkyl, oxo or halo groups, bonded to one of the ring members, referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups may be substituted or unsubstituted. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O⁻. A “hydroxyalkyl” group is a hydroxyl-substituted alkyl group, such as HO—CH₂—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “isocyano” refers to —NC.

The term “isothiocyano” refers to —NCS.

The term “pentafluorosulfanyl” refers to —SF₅.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

As understood by one of ordinary skill in the art, “molecular weight” (also known as “relative molar mass”) is a dimensionless quantity but is converted to molar mass by multiplying by 1 gram/mole or by multiplying by 1 Da—for example, a compound with a weight-average molecular weight of 5,000 has a weight-average molar mass of 5,000 g/mol and a weight-average molar mass of 5,000 Da.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism, and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

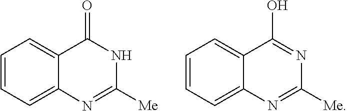

As another example, guanidines may exhibit the following isomeric forms in protic organic solution, also referred to as tautomers of each other:

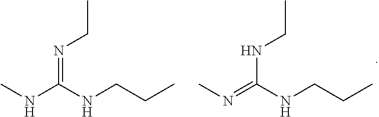

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

Throughout this disclosure, various publications, patents and published patent specifications are referenced by an identifying citation. Also within this disclosure are Arabic numerals referring to referenced citations, the full bibliographic details of which are provided subsequent to the Examples section. The disclosures of these publications, patents and published patent specifications are hereby incorporated by reference into the present disclosure to more fully describe the present technology.

### THE PRESENT TECHNOLOGY

### Compounds of the Present Technology

In an aspect, the present technology provides an epoxyketone compound according to Formula (I)

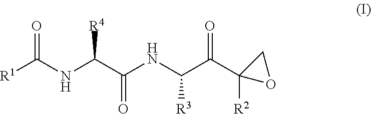

or a stereoisomer or a tautomer thereof, or a pharmaceutically acceptable salt and/or solvate thereof, wherein
   R¹ is alkyl, unsubstituted phenyl, or substituted phenyl;
   R² is H or C₁₋₆ alkyl optionally substituted with OH;
   R³ is C₁₋₆ alkyl optionally substituted with C₂₋₆ alkenyl or phenyl;
   R⁴ is C₁₋₆ alkyl optionally substituted with OH or phenyl.

In any embodiment herein, it may be that an epoxyketone compound according to Formula (I) is not one or more of

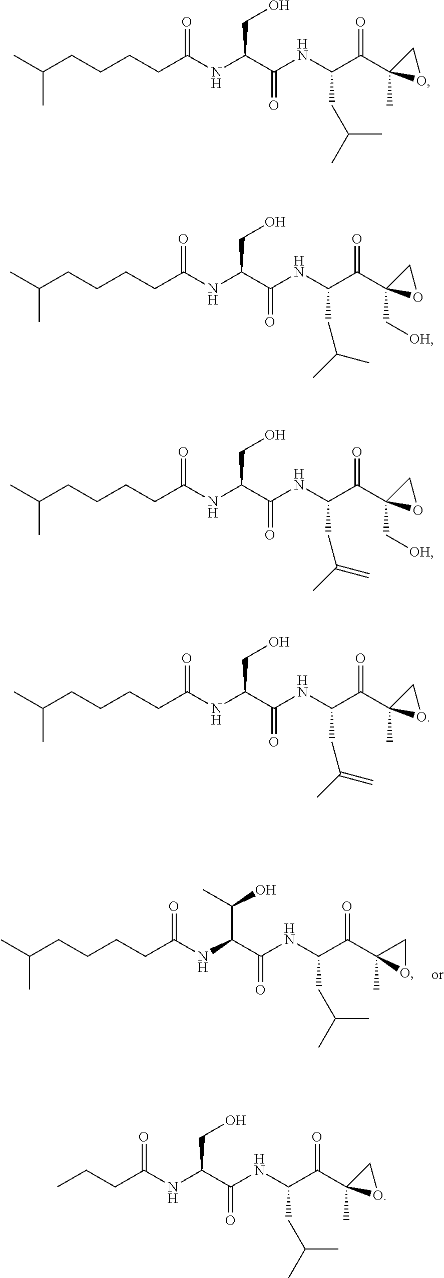 [SMILES: CC(C)CCCCC(=O)N[C@@H](CO)C(=O)N[C@@H](CC(C)C)C(=O)[C@@]1(C)CO1 ; CC(C)CCCCC(=O)N[C@@H](CO)C(=O)N[C@@H](CC(C)C)C(=O)[C@@]1(CO)CO1 ; C=C(C)C[C@H](NC(=O)[C@H](CO)NC(=O)CCCCC(C)C)C(=O)[C@@]1(CO)CO1 ; C=C(C)C[C@H](NC(=O)[C@H](CO)NC(=O)CCCCC(C)C)C(=O)[C@@]1(C)CO1 ; CC(C)CCCCC(=O)N[C@H](C(=O)N[C@@H](CC(C)C)C(=O)[C@@]1(C)CO1)[C@@H](C)O ; CCCC(=O)N[C@@H](CO)C(=O)N[C@@H](CC(C)C)C(=O)[C@@]1(C)CO1]

In any embodiment herein, it may be the epoxyketone compound of Formula (I) is of Formula (Ia)

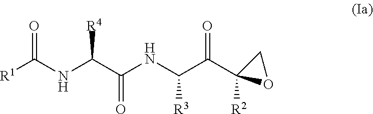

or a pharmaceutically acceptable salt and/or solvate thereof.

In any embodiment herein, it may be that R¹ is unsubstituted phenyl or substituted phenyl. In any embodiment herein, it may be that the epoxyketone compound of Formula (I) is of Formula II

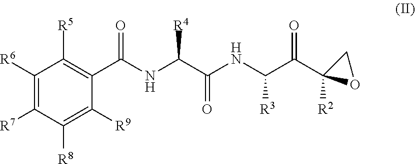

or a pharmaceutically acceptable salt and/or solvate thereof, wherein
R⁵, R⁶, R⁷, R⁸, and R⁹ are each independently selected from —H, —F, —Cl, —Br, —I, alkoxy, —OH, —CF₃, —CN, —COOH, —COOR¹⁰, —C(O)NR¹¹R¹², —CHNOH, —NR¹³R¹⁴, —NHNH₂, —NO₂, —N₃, —NR¹⁵C(O)R¹⁶, —NR¹⁷C(O)OR¹⁸, —OC(O)NR¹⁹R²⁰, —SH, —SR²¹, —S(O)R²², —SO₂R²³, —SO₂OR²⁴, alkyl, heteroalkyl, alkenyl, alkynyl, heterocycloalkyl, aryl, and heteroaryl.

In any embodiment herein, it may be that R⁵, R⁶, R⁷, R⁸, and R⁹ are each independently —H.

In any embodiment herein, it may be that R¹ is alkyl. In some embodiments, R¹ is unsubstituted alkyl (e.g., unsubstituted C₁₋₁₈ alkyl).

In any embodiment herein, it may be that R⁴ is unsubstituted C₁₋₆ alkyl. In some embodiments, R⁴ is C₁₋₆ alkyl substituted with a phenyl group.

In any embodiment herein, it may be that the epoxyketone compound of Formula (I) is of Formula (III)

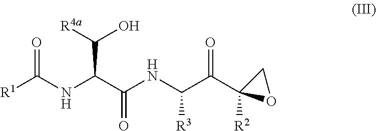

or a pharmaceutically acceptable salt and/or solvate thereof, wherein
R¹ is unsubstituted C₁₋₁₈ alkyl; and
R^{4a} is H or C₁₋₆ alkyl.

In any embodiment herein, it may be that R^{4a} is H. In any embodiment herein, R^{4a} may be unsubstituted C₁₋₆ alkyl.

In any embodiment herein, it may be that the epoxyketone compound (e.g., a compound of Formula (III)) is not one or more of

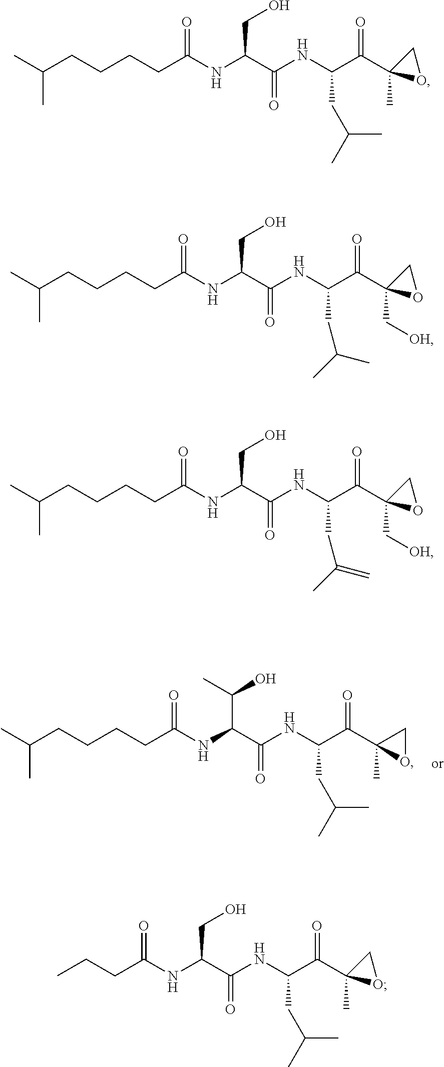 [SMILES: CC(C)CCCCC(=O)N[C@@H](CO)C(=O)N[C@@H](CC(C)C)C(=O)[C@@]1(C)CO1 ; CC(C)CCCCC(=O)N[C@@H](CO)C(=O)N[C@@H](CC(C)C)C(=O)[C@@]1(CO)CO1 ; C=C(C)C[C@H](NC(=O)[C@H](CO)NC(=O)CCCCC(C)C)C(=O)[C@@]1(CO)CO1 ; CC(C)CCCCC(=O)N[C@H](C(=O)N[C@@H](CC(C)C)C(=O)[C@@]1(C)CO1)[C@@H](C)O ; CCCC(=O)N[C@@H](CO)C(=O)N[C@@H](CC(C)C)C(=O)[C@@]1(C)CO1]

In any embodiment herein, it may be that when R² is unsubstituted C₁₋₆ alkyl, R³ is not unsubstituted C₁₋₆ alkyl.

In any embodiment herein, it may be that when R² is hydroxyl substituted C₁₋₆ alkyl, R³ is not unsubstituted C₁₋₆ alkyl or C₁₋₆ alkyl substituted with a C₂₋₆ alkenyl group.

In any embodiment herein, it may be that R² is H, CH₃, or CH₂OH.

In any embodiment herein, it may be that R³ is

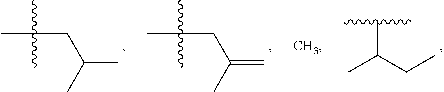

CH(CH₃)₂, or benzyl.

In any embodiment herein, it may be that R⁴ is CH₂OH, benzyl, CH(OH)CH₃, CH(CH₃)₂, or CH₃.

In any embodiment herein, it may be that the epoxyketone compound is a compound of Table A or a pharmaceutically acceptable salt and/or solvate thereof.

TABLE A

| Compound | |
|---|---|
| Number | Structure |
| 12a | 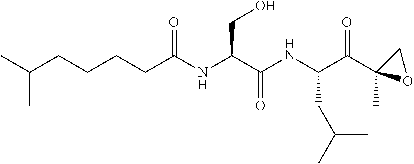 [SMILES: CC(C)CCCCC(=O)N[C@@H](CO)C(=O)N[C@@H](CC(C)C)C(=O)[C@@]1(C)CO1] |
| 12b | 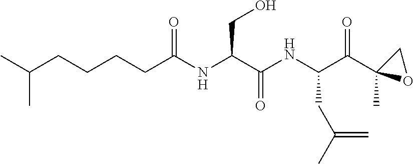 [SMILES: C=C(C)C[C@H](NC(=O)[C@H](CO)NC(=O)CCCCC(C)C)C(=O)[C@@]1(C)CO1] |
| 12c | 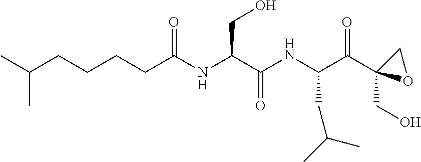 [SMILES: CC(C)CCCCC(=O)N[C@@H](CO)C(=O)N[C@@H](CC(C)C)C(=O)[C@@]1(CO)CO1] |
| 12d | 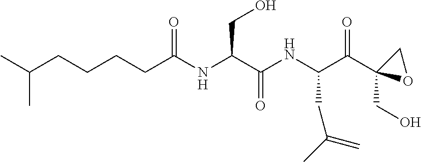 [SMILES: C=C(C)C[C@H](NC(=O)[C@H](CO)NC(=O)CCCCC(C)C)C(=O)[C@@]1(CO)CO1] |
| 12e | 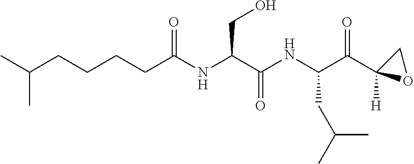 [SMILES: CC(C)CCCCC(=O)N[C@@H](CO)C(=O)N[C@@H](CC(C)C)C(=O)[C@H]1CO1] |
| 12f | 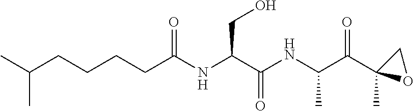 [SMILES: CC(C)CCCCC(=O)N[C@@H](CO)C(=O)N[C@@H](C)C(=O)[C@@]1(C)CO1] |
| 12g | 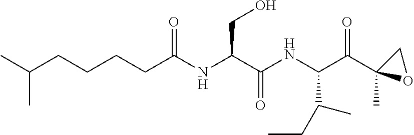 [SMILES: CCC(C)[C@H](NC(=O)[C@H](CO)NC(=O)CCCCC(C)C)C(=O)[C@@]1(C)CO1] |
| 12h | 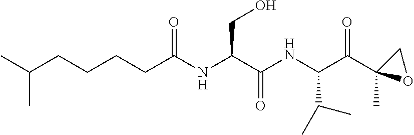 [SMILES: CC(C)CCCCC(=O)N[C@@H](CO)C(=O)N[C@H](C(=O)[C@@]1(C)CO1)C(C)C] |
| 12i | 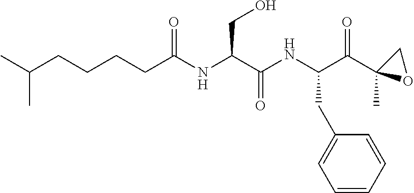 [SMILES: CC(C)CCCCC(=O)N[C@@H](CO)C(=O)N[C@@H](Cc1ccccc1)C(=O)[C@@]1(C)CO1] |
| 12j | 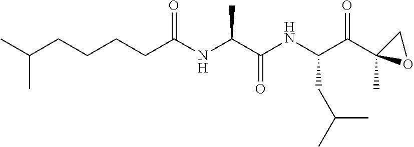 [SMILES: CC(C)CCCCC(=O)N[C@@H](C)C(=O)N[C@@H](CC(C)C)C(=O)[C@@]1(C)CO1] |
| 12k | 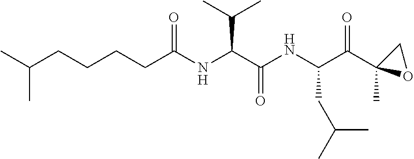 [SMILES: CC(C)CCCCC(=O)N[C@H](C(=O)N[C@@H](CC(C)C)C(=O)[C@@]1(C)CO1)C(C)C] |
| 12l | 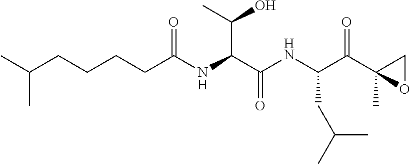 [SMILES: CC(C)CCCCC(=O)N[C@H](C(=O)N[C@@H](CC(C)C)C(=O)[C@@]1(C)CO1)[C@@H](C)O] |
| 12m | 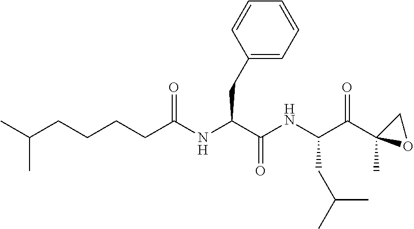 [SMILES: CC(C)CCCCC(=O)N[C@@H](Cc1ccccc1)C(=O)N[C@@H](CC(C)C)C(=O)[C@@]1(C)CO1] |
| 12n | 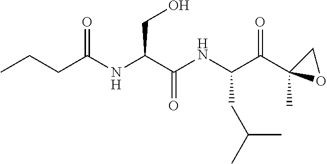 [SMILES: CCCC(=O)N[C@@H](CO)C(=O)N[C@@H](CC(C)C)C(=O)[C@@]1(C)CO1] |
| 12o | 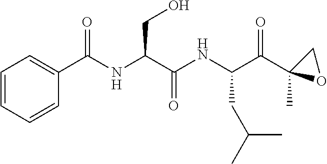 [SMILES: CC(C)C[C@H](NC(=O)[C@H](CO)NC(=O)c1ccccc1)C(=O)[C@@]1(C)CO1] |
| 12p | 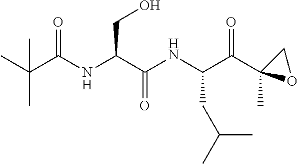 [SMILES: CC(C)C[C@H](NC(=O)[C@H](CO)NC(=O)C(C)(C)C)C(=O)[C@@]1(C)CO1] |
| 12q | 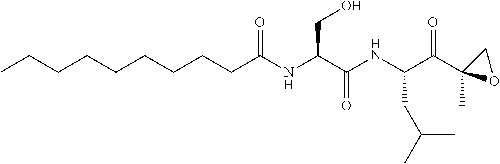 [SMILES: CCCCCCCCCC(=O)N[C@@H](CO)C(=O)N[C@@H](CC(C)C)C(=O)[C@@]1(C)CO1] |
| 12r | 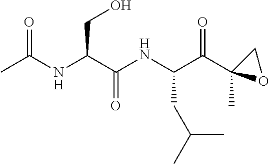 [SMILES: CC(=O)N[C@@H](CO)C(=O)N[C@@H](CC(C)C)C(=O)[C@@]1(C)CO1] |

In another aspect, the present disclosure also provides a β-ketoacid useful in generating an epoxyketone compound of any embodiment described herein, the β-ketoacid being of Formula (A)

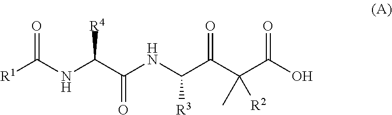

or a stereoisomer or a tautomer thereof, or a pharmaceutically acceptable salt and/or solvate thereof, wherein
R¹ is alkyl, unsubstituted phenyl, or substituted phenyl;
R² is H or C₁₋₆ alkyl optionally substituted with OH;
R³ is C₁₋₆ alkyl optionally substituted with C₂₋₆ alkenyl or phenyl;
R⁴ is C₁₋₆ alkyl optionally substituted with OH or phenyl.

In any embodiment herein of the β-ketoacid, it may be that the β-ketoacid is not one or more of

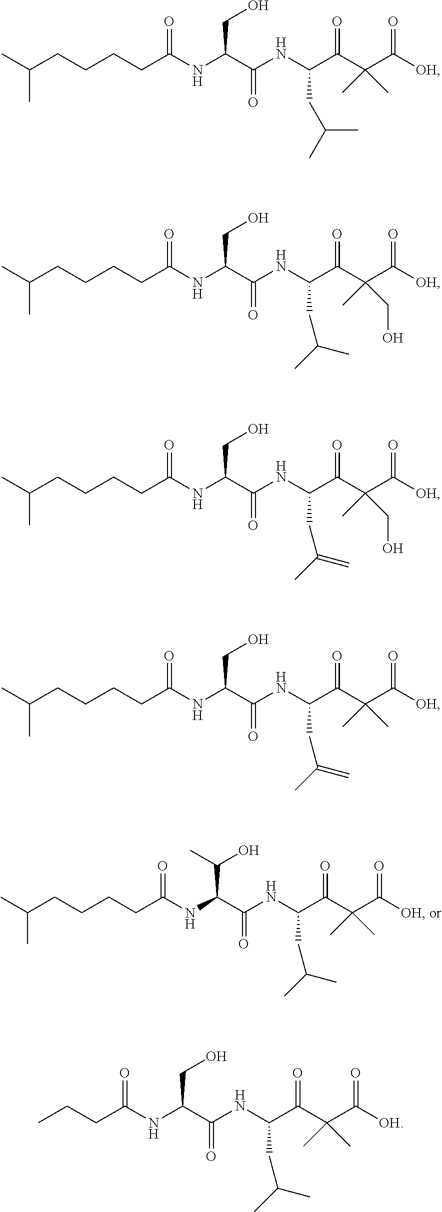

In any embodiment herein of the β-ketoacid, it may be that R¹ is unsubstituted phenyl or substituted phenyl. In any embodiment herein the β-ketoacid, it may be that the β-ketoacid is of Formula (B)

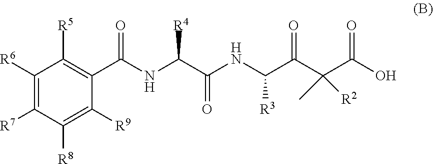

or a pharmaceutically acceptable salt and/or solvate thereof, wherein
R⁵, R⁶, R⁷, R⁸, and R⁹ are each independently selected from —H, —F, —Cl, —Br, —I, alkoxy, —OH, —CF₃, —CN, —COOH, —COOR¹⁰, —C(O)NR¹¹R¹², —CHNOH, —NR¹³R¹⁴, —NHNH₂, —NO₂, —N₃, —NR¹⁵C(O)R¹⁶, —NR¹⁷C(O)OR¹⁸, —OC(O)NR¹⁹R²⁰, —SH, —SR²¹, —S(O)R²², —SO₂R²³, —SO₂OR²⁴, alkyl, heteroalkyl, alkenyl, alkynyl, heterocycloalkyl, aryl, and heteroaryl.

In any embodiment herein of the β-ketoacid, it may be that R⁵, R⁶, R⁷, R⁸, and R⁹ are each independently —H.

In any embodiment herein of the β-ketoacid, it may be that R¹ is alkyl. In some embodiments, R¹ is unsubstituted alkyl (e.g., unsubstituted C₁₋₁₈ alkyl).

In any embodiment herein of the β-ketoacid, it may be that R⁴ is unsubstituted C₁₋₆ alkyl. In some embodiments, R⁴ is C₁₋₆ alkyl substituted with a phenyl group.

In any embodiment herein of the β-ketoacid, it may be that the of the β-ketoacid is of Formula (C)

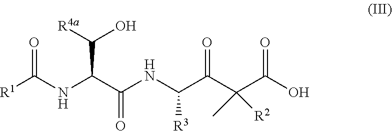

or a pharmaceutically acceptable salt and/or solvate thereof, wherein
R¹ is unsubstituted C₁₋₁₈ alkyl; and
R⁴ is H or C₁₋₆ alkyl.

In any embodiment herein of the β-ketoacid, it may be that R⁴ is H. In any embodiment herein of the β-ketoacid, R⁴ may be unsubstituted C₁₋₆ alkyl.

In any embodiment herein of the β-ketoacid, it may be that when R² is unsubstituted C₁₋₆ alkyl, R³ is not unsubstituted C₁₋₆ alkyl.

In any embodiment herein of the β-ketoacid, it may be that when R² is hydroxyl substituted C₁₋₆ alkyl, R³ is not unsubstituted C₁₋₆ alkyl or C₁₋₆ alkyl substituted with a C₂₋₆ alkenyl group.

In any embodiment herein of the β-ketoacid, it may be that R² is H, CH₃, or CH₂OH.

In any embodiment herein of the β-ketoacid, it may be that R³ is

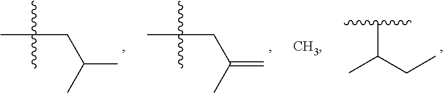

CH(CH₃)₂, or benzyl.

In any embodiment herein of the β-ketoacid, it may be that R⁴ is CH₂OH, benzyl, CH(OH)CH₃, CH(CH₃)₂, or CH₃.

In any embodiment herein, it may be that the β-ketoacid is a compound of Table B or a pharmaceutically acceptable salt and/or solvate thereof.

TABLE B

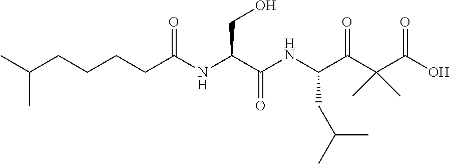 [SMILES: CC(C)CCCCC(=O)N[C@@H](CO)C(=O)N[C@@H](CC(C)C)C(=O)C(C)(C)C(=O)O]

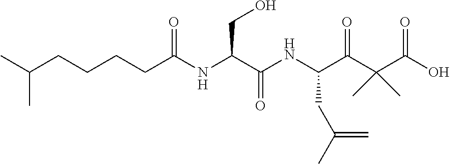 [SMILES: C=C(C)C[C@H](NC(=O)[C@H](CO)NC(=O)CCCCC(C)C)C(=O)C(C)(C)C(=O)O]

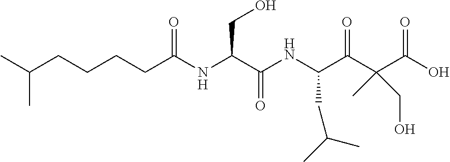 [SMILES: CC(C)CCCCC(=O)N[C@@H](CO)C(=O)N[C@@H](CC(C)C)C(=O)C(C)(CO)C(=O)O]

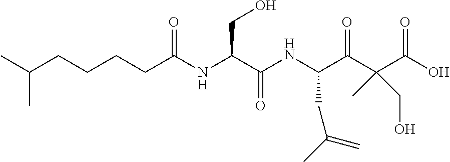 [SMILES: C=C(C)C[C@H](NC(=O)[C@H](CO)NC(=O)CCCCC(C)C)C(=O)C(C)(CO)C(=O)O]

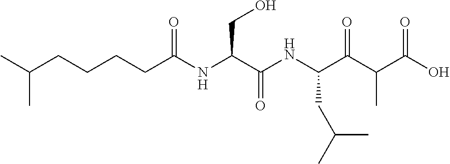 [SMILES: CC(C)CCCCC(=O)N[C@@H](CO)C(=O)N[C@@H](CC(C)C)C(=O)C(C)C(=O)O]

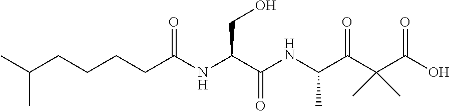 [SMILES: CC(C)CCCCC(=O)N[C@@H](CO)C(=O)N[C@@H](C)C(=O)C(C)(C)C(=O)O]

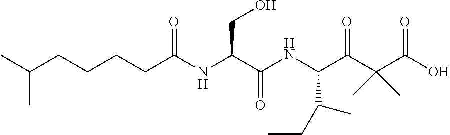 [SMILES: CCC(C)[C@H](NC(=O)[C@H](CO)NC(=O)CCCCC(C)C)C(=O)C(C)(C)C(=O)O]

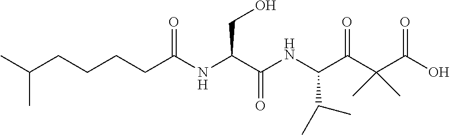 [SMILES: CC(C)CCCCC(=O)N[C@@H](CO)C(=O)N[C@H](C(=O)C(C)(C)C(=O)O)C(C)C]

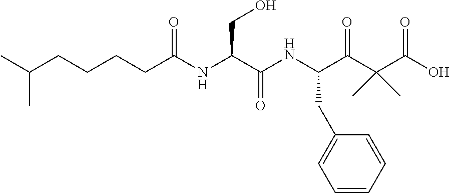 [SMILES: CC(C)CCCCC(=O)N[C@@H](CO)C(=O)N[C@@H](Cc1ccccc1)C(=O)C(C)(C)C(=O)O]

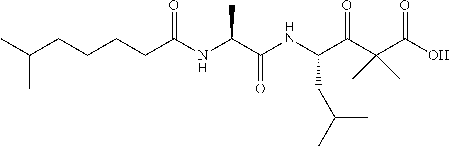 [SMILES: CC(C)CCCCC(=O)N[C@@H](C)C(=O)N[C@@H](CC(C)C)C(=O)C(C)(C)C(=O)O]

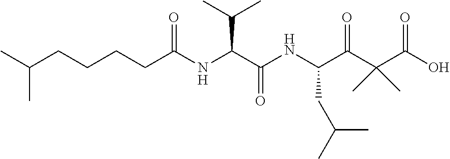 [SMILES: CC(C)CCCCC(=O)N[C@H](C(=O)N[C@@H](CC(C)C)C(=O)C(C)(C)C(=O)O)C(C)C]

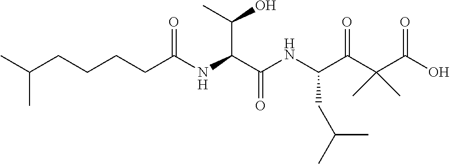 [SMILES: CC(C)CCCCC(=O)N[C@H](C(=O)N[C@@H](CC(C)C)C(=O)C(C)(C)C(=O)O)[C@@H](C)O]

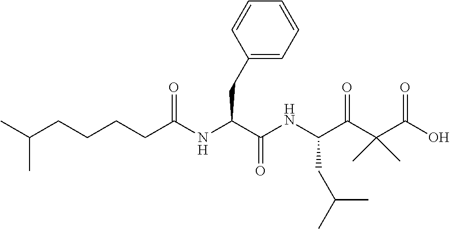 [SMILES: CC(C)CCCCC(=O)N[C@@H](Cc1ccccc1)C(=O)N[C@@H](CC(C)C)C(=O)C(C)(C)C(=O)O]

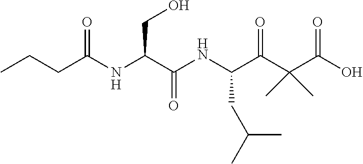 [SMILES: CCCC(=O)N[C@@H](CO)C(=O)N[C@@H](CC(C)C)C(=O)C(C)(C)C(=O)O]

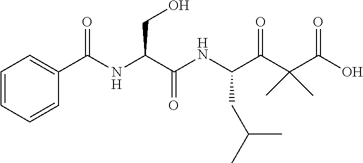 [SMILES: CC(C)C[C@H](NC(=O)[C@H](CO)NC(=O)c1ccccc1)C(=O)C(C)(C)C(=O)O]

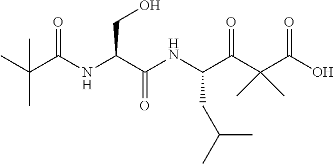 [SMILES: CC(C)C[C@H](NC(=O)[C@H](CO)NC(=O)C(C)(C)C)C(=O)C(C)(C)C(=O)O]

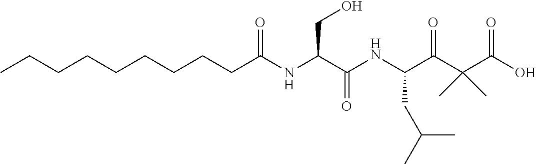 [SMILES: CCCCCCCCCC(=O)N[C@@H](CO)C(=O)N[C@@H](CC(C)C)C(=O)C(C)(C)C(=O)O]

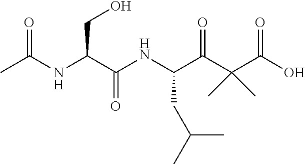 [SMILES: CC(=O)N[C@@H](CO)C(=O)N[C@@H](CC(C)C)C(=O)C(C)(C)C(=O)O]

### Compositions and Methods

For ease of reference, the compounds included in any aspect or embodiment herein (an epoxyketone compound of the present technology and a β-ketoacid of the present technology) may be referred to anywhere in this disclosure as “a compound of the present technology,” “compounds of the present technology,” or the like. Similarly for ease of reference, the compositions, medicaments, and pharmaceutical compositions of the present technology may collectively be referred to herein as “compositions,” “compositions of the present technology,” or the like.

In an aspect, a composition is provided that includes a compound of any aspect or embodiment disclosed herein as well as a pharmaceutically acceptable carrier or one or more excipients, fillers or agents (collectively referred to hereafter as “pharmaceutically acceptable carrier” unless otherwise indicated and/or specified). In a related aspect, a medicament for treating a disease described herein (e.g. a disease associated with proteasome activity such as cancer (e.g., hematopoietic cancer, acute lymphoblastic leukemia (ALL), or multiple myeloma)) in a subject is provided that includes a compound of any embodiment disclosed herein and optionally a pharmaceutically acceptable carrier. The medicament of any embodiment herein may include an effective amount of the compound for treating the disease described herein. In a related aspect, a pharmaceutical composition is provided that includes (i) an effective amount of a compound of any embodiment disclosed herein, wherein the effective amount of the compound may be effective to treat a disease described herein (e.g. a disease associated with proteasome activity such as cancer (e.g., hematopoietic cancer, acute lymphoblastic leukemia (ALL), or multiple myeloma)); and (ii) a pharmaceutically acceptable carrier. In any embodiment herein, the disease may be cancer (e.g., hematopoietic cancer, acute lymphoblastic leukemia (ALL), or multiple myeloma).

“Effective amount” refers to the amount of a compound or composition required to produce a desired effect. One example of an effective amount includes amounts or dosages that yield acceptable toxicity and bioavailability levels for therapeutic (pharmaceutical) use including, but not limited to, reduction of a tumor mass. In any aspect or embodiment disclosed herein (collectively referred to herein as “any embodiment herein,” “any embodiment disclosed herein,” or the like) of the compositions, pharmaceutical compositions, and methods including compounds of the present technology, the effective amount may be an amount effective in treating a disease described herein (e.g. a disease associated with proteasome activity such as cancer (e.g., hematopoietic cancer, acute lymphoblastic leukemia (ALL), or multiple myeloma)). By way of example, the effective amount of any embodiment herein including a compound of the present technology may be from about 0.01 μg to about 200 mg of the compound (such as from about 0.1 μg to about 50 mg of the compound or about 10 μg to about 20 mg of the compound). The methods and uses according to the present technology may include an effective amount of a compound of any embodiment disclosed herein. In any aspect or embodiment disclosed herein, the effective amount may be determined in relation to a subject. As used herein, a “subject” or “patient” is a mammal, such as a cat, dog, rodent or primate. Typically the subject is a human, and, preferably, a human suffering from or suspected of suffering from pain. The term “subject” and “patient” can be used interchangeably.

Thus, the instant present technology provides pharmaceutical compositions and medicaments including a compound of any embodiment disclosed herein (or a composition of any embodiment disclosed herein) and a pharmaceutically acceptable carrier. The compositions may be used in the methods and treatments described herein. The pharmaceutical composition may be packaged in unit dosage form. The unit dosage form may be effective in treating a disease described herein (e.g. a disease associated with proteasome activity such as cancer (e.g., hematopoietic cancer, acute lymphoblastic leukemia (ALL), or multiple myeloma)). The unit dosage form may be effective in treating a tumor by reducing a tumor volume when administered to a subject in need thereof. Generally, a unit dosage including a compound of the present technology will vary depending on patient considerations. Such considerations include, for example, age, protocol, condition, sex, extent of disease, contraindications, concomitant therapies and the like. An exemplary unit dosage based on these considerations may also be adjusted or modified by a physician skilled in the art. For example, a unit dosage for a patient comprising a compound of the present technology may vary from 1×10⁻⁴ g/kg to 1 g/kg, preferably, 1×10⁻³ g/kg to 1.0 g/kg. Dosage of a compound of the present technology may also vary from 0.01 mg/kg to 100 mg/kg or, preferably, from 0.1 mg/kg to 10 mg/kg. Suitable unit dosage forms, include, but are not limited to parenteral solutions, oral solutions, powders, tablets, pills, gelcaps, capsules, lozenges, suppositories, patches, nasal sprays, injectables, implantable sustained-release formulations, mucoadherent films, topical varnishes, lipid complexes, liquids, etc.

The pharmaceutical compositions and medicaments may be prepared by mixing one or more compounds and/or compositions of the present technology with pharmaceutically acceptable carriers, excipients, binders, diluents or the like. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars and/or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts until, for example, there is a reduction in the mass of a tumor in a subject. The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the B-cell malignancy (e.g., non-Hodgkin lymphoma or chronic lymphocytic leukemia) associated with the tumor, and the pharmacological activity of the compound being used. The determination of optimum dosages for a particular patient is well known to those skilled in the art.

Various assays and model systems can be readily employed to determine the therapeutic effectiveness of the treatment according to the present technology. Effectiveness of the compositions (as well as determination of effective amounts) and methods of the present technology may also be demonstrated by a decrease in the mass of a tumor and/or slowing the growth of a tumor and/or affecting an increase in the therapeutic responsiveness of a B-cell malignancy to an anti-CD20 monoclonal antibody.

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

The compounds of the present technology can also be administered to a patient along with other conventional therapeutic agents that may be useful in the treatment of tumors or in vaccination. The administration may include oral administration, parenteral administration, or nasal administration. In any of these embodiments, the administration may include intratumoral injections, subcutaneous injections, intravenous injections, intraperitoneal injections, or intramuscular injections. In any of these embodiments, the administration may include oral administration. The methods of the present technology can also include administering, either sequentially or in combination with one or more compounds of the present technology, a conventional therapeutic agent in an amount that can potentially or synergistically be effective for the treatment of a disease described herein (e.g. a disease associated with proteasome activity such as cancer (e.g., hematopoietic cancer, acute lymphoblastic leukemia (ALL), or multiple myeloma)).

In an aspect, a compound of the present technology is administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

In an aspect, the present technology provides a method of treating a disease where the method includes administering to a subject an effective amount of a β-ketoacid of any embodiment disclosed herein and administering to the subject an effective amount of an epoxyketone synthase. Suitable epoxyketone synthases include those disclosed in Huang, C. “Parallelized gene cluster editing illuminates mechanisms of epoxyketone proteasome inhibitor biosynthesis” Nucleic Acids Research 2023, 51(3), 1488-1499; Zabala, D. et al. “A flavin-dependent decarboxylase-dehydrogenase-monooxygenase assembles the warhead of α,β-epoxyketone proteasome inhibitors” J. Am. Chem. Soc. 2016, 138, 4342-4345; Keller, L. et al. (2015) Macyranones: Structure, biosynthesis, and binding mode of an unprecedented epoxyketone that targets the 20S proteasome. J. Am. Chem. Soc. 2015, 137, 8121-8130; and Owen, J. G. et al. “Multiplexed metagenome mining using short DNA sequence tags facilitates targeted discovery of epoxyketone proteasome inhibitors” Proc. Natl. Acad. Sci. U.S.A. 2015, 112, 4221-4226; as well as recombinant forms thereof. The disease may associated with proteasome activity, and in any embodiment herein the disease may be cancer (e.g., a hematopoietic cancer, acute lymphoblastic leukemia (ALL), and/or multiple myeloma). In any embodiment herein, the method may include administering the epoxyketone synthase locally to a tumor comprising the cancer and/or may include administering the β-ketoacid locally to a tumor comprising the cancer. In any embodiment herein, the method may include intratumoral injection of the epoxyketone synthase and/or the β-ketoacid. In any embodiment herein, the epoxyketone synthase may be EpnF, EpxF, TmcF, MynC, AR412 ACAD, AR456 ACAD, AB162 ACAD, NM1663 ACAD, AZ379 ACAD, AB9 ACAD, AB1383 ACAD, AB1241 ACAD, and/or AZ40 ACAD, or a recombinant variant thereof.

In another aspect, the present technology provides methods of identifying a target of interest including contacting the target of interest with a detectable or imaging effective quantity of a labeled compound of the present technology. A detectable or imaging effective quantity is a quantity of a labeled compound of the present technology necessary to be detected by the detection method chosen. For example, a detectable quantity can be an administered amount sufficient to enable detection of binding of the labeled compound to a target of interest. Suitable labels are known by those skilled in the art and can include, for example, radioisotopes, radionuclides, isotopes, fluorescent groups, biotin (in conjunction with streptavidin complexation), and chemiluminescent groups. Upon binding of the labeled compound to the target of interest, the target may be isolated, purified and further characterized such as by determining the amino acid sequence.

The terms “associated” and/or “binding” can mean a chemical or physical interaction, for example, between a compound of the present technology and a target of interest. Examples of associations or interactions include covalent bonds, ionic bonds, hydrophilic-hydrophilic interactions, hydrophobic-hydrophobic interactions and complexes. Associated can also refer generally to “binding” or “affinity” as each can be used to describe various chemical or physical interactions. Measuring binding or affinity is also routine to those skilled in the art. For example, compounds of the present technology can bind to or interact with a target of interest or precursors, portions, fragments and peptides thereof and/or their deposits.

As indicated previously in this disclosure, in an aspect a method of treating a subject suffering from a disease associated with proteasome activity such as cancer (e.g., hematopoietic cancer, acute lymphoblastic leukemia (ALL), or multiple myeloma) is provided, where the method includes administering to the subject an effective amount of a compound of any embodiment disclosed herein or administering a composition of any embodiment disclosed herein. In any embodiment herein of the method, the administering may include intratumoral administration. In any embodiment herein, the disease may be hematopoietic cancer, acute lymphoblastic leukemia (ALL), or multiple myeloma.

In any embodiment herein, the administering may include local administration of the compound to a site in the subject including the cancer or local administration of the composition to a site in the subject including the cancer. In any embodiment herein, the administering may include oral, rectal, nasal, vaginal, transdermal, intravenous, intramuscular, or inhalation administration. In any embodiment herein, the administering may include injection of the compound into the site in the subject including the cancer or proximal to the site in the subject including the cancer.

In another aspect, the present disclosure also provides a process for preparing an epoxyketone compound of any embodiment disclosed herein, the process comprising contacting a β-ketoacid of any embodiment disclosed herein with an epoxyketone synthase to provide the epoxyketone compound. In any embodiment herein, the contacting may occur at a temperature of about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., or about 45° C., or any range including and/or in between any two of the foregoing values. In any embodiment herein, the contacting may occur at a temperature of about 37° C. In any embodiment herein, the contacting may occur at a pH of about 7 to about 8, including about 7.0, about 7.2, about 7.4, about 7.6, about 7.8, about 8.0, or any range including and/or in between any two of the foregoing values. In any embodiment herein, the contacting may occur at a pH of about 7.5. In any embodiment herein, the contacting may occur in a buffer solution such as a tris(hydroxymethyl)aminomethane (also referred to as “Tris”) solution. In any embodiment herein, the epoxyketone synthase may be EpnF, EpxF, TmcF, MynC, AR412 ACAD, AR456 ACAD, AB162 ACAD, NM1663 ACAD, AZ379 ACAD, AB9 ACAD, AB1383 ACAD, AB1241 ACAD, and/or AZ40 ACAD, or a recombinant variant thereof.

The examples herein are provided to illustrate advantages of the present technology and to further assist a person of ordinary skill in the art with preparing or using the compounds and compositions of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects, or embodiments of the present technology.

### EXAMPLES

### Example 1: Exemplary Synthetic Procedures and Characterization of the Compounds

Reagents. Reagents were obtained from Aldrich Chemical or Acros Organics and used without further purification. Optima or HPLC grade solvents were obtained from Fisher Scientific, degassed with Ar, and purified on a solvent drying system unless otherwise indicated.

Reactions. All reactions were performed in flame-dried glassware under positive Ar pressure with magnetic stirring unless otherwise noted. Liquid reagents and solutions were transferred thru rubber septa via syringes flushed with Ar prior to use. Cold baths were generated as follows: 0° C., wet ice/water −78° C., dry ice/acetone.

Chromatography. TLC was performed on 0.25 mm E. Merck silica gel 60 F254 plates and visualized under UV light (254 nm) or by staining with potassium permanganate (KMnO₄), Seebach's staining, or p-anisaldehyde. Chromatography was performed on an ISCO CombiFlash Next Gen 300+ instrument with RediSep silica gel normal-phase columns using variable flow rates depending on column size and a gradient of 0-100% EtOAc in hexanes or 0-10% MeOH in CH₂Cl₂ with UV detection at 254 nm. Analytical to semi-preparative scale HPLC was carried out on a Waters Alliance 2695 HPLC with 2996 diode array detector with a Higgins Analytical Targa C18 reverse phase column (5 cm×4.6 mm, 3 μm, 120 Å), using a flow rate of 1.0 mL/min and a gradient of 5-95% CH₃CN in 0.1% aq TFA over 5 min with UV detection at 254 nm. Preparative scale HPLC purification was carried out on a Waters 2545 HPLC with 2996 diode array detector using a Waters xBridge BEH C18 OBD Prep Column, (25 cm×19 mm, 5 μm, 130 Å) using a flow rate of 15.0 mL/min and a gradient of 5-95% CH₃CN in 0.1% aq TFA over 20 min with UV detection at 254 nm.

Analytical LC-MS was carried out on a Waters Acuity SQD LC-MS in electrospray ionization (ESI) mode, with a Waters Acuity UPLC BEH C18 reverse-phase column (10 cm×2.1 mm, 1.7 μm, 130 Å), using a flow rate of 0.3 mL/min and a gradient of 5-95% CH3CN in 0.1% aq TFA over 8 min. Lyophilization of larger aqueous samples was performed using a Labconco Freezone 2.5 instrument.

Analytical Instrumentation. IR spectra were recorded on a Bruker Optics Tensor 27 FTIR spectrometer using an attenuated total reflection (ATR) attachment with peaks reported in cm⁻¹. NMR spectra were recorded on a Bruker UltraShield Plus 500 MHz Avance III NMR or UltraShield Plus 600 MHz Avance III NMR with DCH CryoProbe at 24° C. in CDCl₃ unless otherwise indicated. Chemical shifts are expressed in ppm relative to TMS (¹H, 0 ppm) or solvent signals: CDCl₃ (¹H, 7.26 ppm, ¹³C, 77.0 ppm), CD₃OD (¹H, 3.31, ¹³C, 49.00), (CD₃)₂SO (¹H, 2.50 ppm, ¹³C, 39.52 ppm); coupling constants are expressed in Hz. NMR spectra were processed using Bruker TopSpin, Mnova (www.mestrelab.com/software/mnova-nmr), or nucleomatica iNMR (www.inmr.net) software.

Mass spectra were obtained on a Waters Acuity SQD LC-MS or PE SCIEX API-100 by electrospray (ESI) ionization or atmospheric pressure chemical ionization (AP-CI). High-resolution mass spectra were obtained on a Waters Acuity Premiere XE TOF LC-MS by electrospray ionization (ESI).

### Synthesis of β-Ketoacid Substrates

Synthesis of α,α-Dimethyl-β-ketoacid Substrates (11a, 11b). Compounds 11a and 11b were prepared according to below scheme.

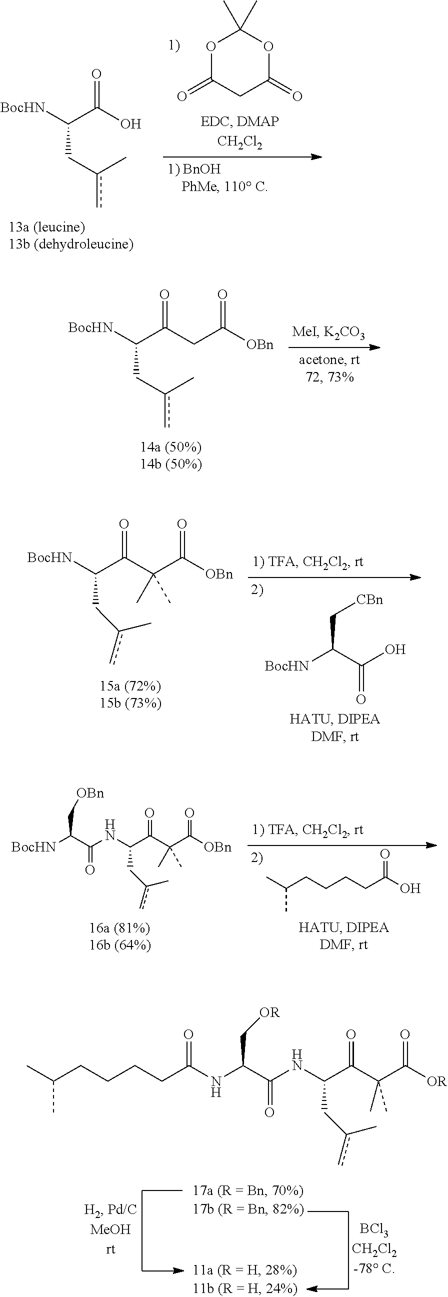

### Synthesis of Boc-Protected β-Ketoester Fragments (14a, 14b)

General Protocol. In a roundbottom flask, Boc-protected amino acid 13a or 13b (1 equiv), DMAP (1.1 equiv), and Meldrum's acid (1.1 equiv) were suspended in anhyd CH₂Cl₂ (0.1 M), then EDC (3 equiv) was added portion-wise and the reaction was stirred until complete conversion as determined by TLC analysis. The reaction mixture was poured over 1 N HCl and extracted with CH₂Cl₂ (2×). The combined organic extracts were washed with brine, dried (NaSO₄), filtered, and concentrated by rotary evaporation. The crude Meldrum's acid adduct and benzyl alcohol (1.1 equiv) were dissolved in anhyd toluene (0.1 M) and heated to 110° C. for 4 h, then concentrated by rotary evaporation. Purification by silica flash chromatography (9:1 hexanes/EtOAc) afforded the O-benzyl β-ketoester 14a or 14b.

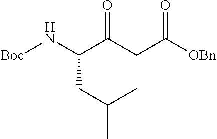 [SMILES: CC(C)C[C@H](NC(=O)OC(C)(C)C)C(=O)CC(=O)OCc1ccccc1]

Benzyl (4S)-4-{[(tert-butoxy)carbonyl]amino}-6-methyl-3-oxoheptanoate (14a). 7.6 g (48% over two steps), white crystalline solid. TLC: R_{f} 0.4 (9:1 hexanes/EtOAc). IR (ZnSe) 3378.98; 3070.35; 3034.93; 2978.10; 2934.29; 1745.33; 1709.69; 1498.28; 1455.38; 1392.31; 1367.24; 1315.84; 1248.44; 1163. ¹H NMR (500 MHz, CDCl₃) δ 7.41-7.30 (m, 5H), 5.23-5.14 (m, 2H), 4.97 (d, J=7.1 Hz, 1H), 4.87 (s, 1H), 4.75 (s, 1H), 4.39 (s, 1H), 3.70 (d, J=16.1 Hz, 1H), 3.60 (d, J=16.0 Hz, 1H), 2.59-2.51 (m, 1H), 2.29-2.21 (m, 1H), 1.73 (s, 3H), 1.44 (s, 9H). ¹³C NMR (126 MHz, CDCl₃) δ 202.96, 166.92, 155.62, 135.38, 128.58, 128.40, 128.37, 79.97, 67.07, 58.26, 46.16, 39.52, 28.30, 24.75, 23.21, 21.50. HRMS (ESI) m/z calcd for C₂₀H₂₉NO₅ Na ([M+Na]+) 386.1943. found 386.1935.

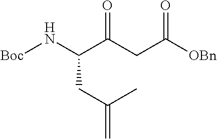 [SMILES: C=C(C)C[C@H](NC(=O)OC(C)(C)C)C(=O)CC(=O)OCc1ccccc1]

Benzyl (S)-4-((tert-butoxycarbonyl)amino)-6-methyl-3-oxohept-6-enoate (14b). 4.3 g (50% over two steps), white crystalline solid. TLC: R_{f} 0.4 (9:1 hexanes/EtOAc). IR (ZnSe) 3378.98; 3070.35; 3034.93; 2978.10; 2934.29; 1745.33; 1709.69; 1498.28; 1455.38; 1392.31; 1367.24; 1315.84; 1248.44; 1163. ¹H NMR (500 MHz, CDCl₃) δ 7.41-7.30 (m, 5H), 5.23-5.14 (m, 2H), 4.97 (d, J=7.1 Hz, 1H), 4.87 (s, 1H), 4.75 (s, 1H), 4.39 (s, 1H), 3.70 (d, J=16.1 Hz, 1H), 3.60 (d, J=16.0 Hz, 1H), 2.59-2.51 (m, 1H), 2.29-2.21 (m, 1H), 1.73 (s, 3H), 1.44 (s, 9H). ¹³C NMR (126 MHz, CDCl₃) δ 202.39, 166.90, 155.54, 140.56, 135.33, 128.61, 128.45, 128.40, 128.23, 114.68, 80.23, 67.15, 57.75, 46.33, 38.97, 28.31, 28.26, 21.78. HRMS (ESI) m/z calcd for C₂₀H₂₇NO₅ Na ([M+Na]⁺) 384.1787. found 384.1779.

### Dimethylation of β-Ketoesters (15a, 15b)

General Protocol. In a roundbottom flask, β-ketoester 14a or 14b (1 equiv) and K₂CO₃ (2.1 equiv) were suspended in anhyd acetone (0.1 M). Iodomethane (9 equiv) was added dropwise via syringe and the reaction was stirred at rt overnight until complete conversion as determined by TLC analysis. Water was added to the mixture and the aq layer was extracted with EtOAc (3×). The combined organic extracts were washed with brine, dried (Na₂SO₄), filtered, and concentrated by rotary evaporation. Purification by silica flash chromatography (9:1 hexanes/EtOAc) afforded the α,α-dimethyl-β-ketoester 15a or 15b.

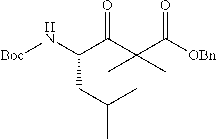 [SMILES: CC(C)C[C@H](NC(=O)OC(C)(C)C)C(=O)C(C)(C)C(=O)OCc1ccccc1]

Benzyl (4S)-4-{[(tert-butoxy)carbonyl]amino}-2,2,6-trimethyl-3-oxoheptanoate (15a). 400 mg (72%), colorless oil. TLC: R_{f} 0.89 (2:1 hexanes/EtOAc) IR (ZnSe, ATR): 3382.18; 2959.37; 2871.30; 1706.02; 1499.00; 1455.95; 1366.91; 1160.92; 1144.20; 1042.70; 1026.00 ¹H NMR (500 MHz, CDCl₃): δ 7.38-7.28 (m, 4H), 5.16 (q, J=12.4 Hz, 2H), 4.78 (d, J=9.7 Hz, 1H), 4.64 (td, J=10.3, 3.1 Hz, 1H), 1.66-1.57 (m, 1H), 1.48-1.39 (m, 16H), 1.29 (dq, J=14.3, 5.8, 3.9 Hz, 1H), 0.89 (d, J=6.5 Hz, 3H), 0.85 (d, J=6.6 Hz, 3H). ¹³C NMR (126 MHz): δ 208.63, 172.79, 155.13, 135.44, 128.62, 128.36, 128.19, 79.65, 66.99, 55.05, 54.63, 54.13, 41.80, 41.56, 28.26, 24.60, 24.27, 23.42, 22.38, 22.00, 21.23. HRMS (ESI) m/z calcd for C₂₂H₃₃NO₅ ([M+Na]⁺) 414.2256. found 414.2236.

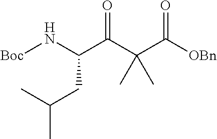 [SMILES: CC(C)C[C@H](NC(=O)OC(C)(C)C)C(=O)C(C)(C)C(=O)OCc1ccccc1]

Benzyl (4S)-4-{[(tert-butoxy)carbonyl]amino}-2,2,6-trimethyl-3-oxohept-6-enoate (15b). 405 mg (73%), colorless oil. TLC: R_{f} 0.85 (2:1 hexanes/EtOAc). IR (ZnSe) 2980.92; 2930.17; 1713.43; 1499.65; 1456.57; 1392.08; 1368.30; 1250.49; 1216.53; 1040.50; 908.95; 858.03 ¹H NMR (500 MHz, CDCl₃) δ 7.35 (qd, J=5.8, 2.8 Hz, 5H), 5.27-5.04 (m, 2H), 4.77 (s, 1H), 4.70 (td, J=9.9, 3.9 Hz, 1H), 4.62 (s, 1H), 4.53 (d, J=9.4 Hz, 1H), 2.46 (dd, J=14.4, 3.8 Hz, 1H), 2.02 (dd, J=14.3, 10.3 Hz, 1H), 1.67 (s, 3H), 1.56 (d, J=5.5 Hz, 1H), 1.43 (d, J=11.1 Hz, 6H), 1.39 (s, 8H). ¹³C NMR (126 MHz, CDCl₃) δ 172.96, 140.80, 128.72, 128.47, 128.30, 114.25, 79.90, 67.07, 54.60, 54.12, 40.76, 28.23, 22.59, 21.85, 21.78. HRMS (ESI) m/z calcd for C₂₂H₃₁NO₅ Na ([M+Na]⁺) 412.2100. found 412.2099.

Boc Deprotection of β-Ketoesters and Coupling with Protected Serine (16a,16b).

General Protocol. In a roundbottom flask, Boc-protected β-ketoester 15a or 15b (1 equiv) was dissolved in a mixture of anhyd CH₂Cl₂ and trifluoroacetic acid (4:1) and the reaction was stirred until complete conversion as determined by LC-MS analysis. The mixture was concentration by rotary evaporation and the crude amine was carried forward directly to the next step without further purification.

In a glass vial, the crude amine was dissolved in DMF (0.2 M) and cooled at 0° C. In a roundbottom flask, O-benzyl-N-(tert-butoxycarbonyl)-L-serine (1.1 equiv) and HATU (1.1 equiv) were suspended in anhyd DMF (0.2 M) and stirred at 0° C. for 10 min. The crude amine solution in DMF was added dropwise to the stirring solution of HATU and O-benzyl-N-(tert-butoxycarbonyl)-L-serine (0.1 M final conc) and stirred for 10 min. N,N-Diisopropylethylamine (3 equiv) was added dropwise. The reaction was allowed to warm to rt and stirred until complete conversion as determined by LC-MS analysis. The mixture was diluted with EtOAc and H₂O, the layers were separated, and the aq layer was extracted with EtOAc (2×). The combined organic extracts were washed with satd NaHCO₃, H₂O, and brine, dried (Na₂SO₄), filtered, and concentrated by rotary evaporation. Purification by silica flash chromatography (7:3 hexanes/EtOAc) afforded the dipeptide β-ketoester 16a or 16b.

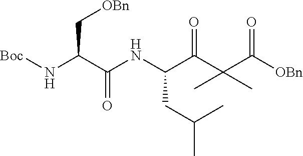 [SMILES: CC(C)C[C@H](NC(=O)[C@H](COCc1ccccc1)NC(=O)OC(C)(C)C)C(=O)C(C)(C)C(=O)OCc1ccccc1]

Benzyl (4S)-4-[(2S)-3-(benzyloxy)-2-([(tert-butoxy)carbonyl]amino)propanamido]-2,2,6-trimethyl-3-oxoheptanoate (16a). 460 mg (81%), colorless oil. TLC: R_{f} 0.58 (1:2 hexanes/EtOAc) IR (ZnSe, ATR): 3338.41; 2975.59; 2932.79; 2869.87; 2251.67; 1713.94; 1676.22; 1497.08; 1470.16; 1454.74; 1390.50; 1366.34; 1250.03; 1211.23; 1160.87; 1111.32; 1045.89; 1025.99 ¹H NMR (500 MHz, CDCl₃): δ 7.41-7.28 (m, 10H), 6.85 (d, J=9.3 Hz, 1H), 5.40-5.35 (m, 1H), 5.21 (d, J=12.4 Hz, 1H), 5.15 (d, J=12.4 Hz, 1H), 5.05 (q, J=7.9 Hz, 1H), 4.54 (s, 2H), 4.25 (s, 1H), 3.90 (dd, J=9.2, 3.8 Hz, 1H), 3.50 (dd, J=9.2, 6.5 Hz, 1H), 1.57 (dt, J=13.4, 6.6 Hz, 1H), 1.48 (s, 3H), 1.47 (s, 9H), 1.44 (s, 3H), 1.37 (t, J=8.0, 6.0 Hz, 2H), 0.89 (d, J=6.5 Hz, 3H), 0.81 (d, J=6.6 Hz, 3H). ¹³C NMR (126 MHz, CDCl₃) δ 207.60, 172.74, 169.70, 155.42, 137.37, 135.42, 128.61, 128.54, 128.48, 128.37, 128.18, 127.94, 127.87, 80.29, 73.56, 69.77, 67.28, 55.12, 53.90, 52.32, 41.57, 28.28, 24.56, 23.45, 22.21, 22.12, 21.21. HRMS (ESI) m/z calcd for C₃₂H₄₅N₂O₇ ([M+H]⁺) 569.3227. found 569.3224.

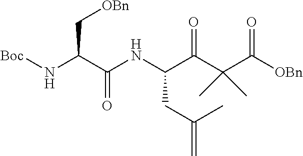 [SMILES: C=C(C)C[C@H](NC(=O)[C@H](COCc1ccccc1)NC(=O)OC(C)(C)C)C(=O)C(C)(C)C(=O)OCc1ccccc1]

Benzyl (S)-4-[(S)-3-(benzyloxy)-2-([tert-butoxycarbonyl]amino)propanamido]-2,2,6-trimethyl-3-oxohept-6-enoate (16b). 96 mg (64%), colorless oil. TLC: R_{f} 0.72 (2:1 hexanes/EtOAc). IR (ZnSe) 3324.35; 3066.54; 3033.31; 2978.50; 2934.57; 2869.13; 1714.07; 1676.39; 1497.14; 1454.57; 1390.82; 1366.37; 1248.64; 1162.92; 1145.78; 1105.96; 1043.11; 1026.57; 992.45; 955.92; 905.78; 861.80 ¹H NMR (500 MHz, CDCl₃) δ 7.41-7.24 (m, 10H), 6.85 (d, J=9.3 Hz, 1H), 5.41-5.36 (m, 1H), 5.20-5.10 (m, 2H), 5.04 (q, J=7.8 Hz, 1H), 4.56-4.42 (m, 3H), 4.24 (s, 1H), 3.88 (dd, J=9.2, 3.8 Hz, 1H), 3.50 (dd, J=9.2, 6.4 Hz, 1H), 1.57 (td, J=14.5, 13.5, 7.8 Hz, 1H), 1.46 (d, J=5.0 Hz, 14H), 1.44-1.34 (m, 4H), 1.27 (q, J=7.2 Hz, OH), 0.88 (d, J=6.4 Hz, 3H), 0.80 (d, J=6.6 Hz, 3H). ¹³C NMR (126 MHz, CDCl₃) δ 206.97, 172.80, 169.69, 140.23, 137.40, 135.38, 128.67, 128.62, 128.46, 128.40, 128.30, 128.19, 127.93, 127.90, 114.43, 73.47, 69.61, 67.31, 55.19, 53.61, 52.11, 40.53, 28.28, 22.25, 22.16, 21.74, 21.71. HRMS (ESI) m/z calcd for C₃₂H₄₂N₂O₇ Na ([M+Na]⁺) 589.2890. found 589.2873.

### Boc Deprotection of Dipeptides and N-Acylation (17a, 17b)

General Protocol. In a roundbottom flask, Boc-protected dipeptide β-ketoester 16a or 16b (1 equiv) was dissolved in a mixture of anhyd CH₂Cl₂ and trifluoroacetic acid (4:1) and the reaction was stirred until complete conversion as determined by LC-MS analysis. The solvent was removed by rotary evaporation and the crude amine was carried forward directly to the next step without further purification.

In a glass vial, the crude amine was dissolved in DMF (0.2 M) and cooled to 0° C. 6-Methylheptanoic acid (1.1 equiv) and HATU (1.1 equiv) were suspended in anhyd DMF (0.2 M). The crude amine solution was added dropwise to the stirring solution of HATU and 6-methylheptanoic acid (0.1 M final conc) and stirred for 10 min. N,N-Diisopropylethylamine (3 equiv) was added dropwise. The reaction was allowed to warm to rt and stirred until complete conversion as determined by LC-MS analysis. The mixture was diluted with EtOAc and H₂O and separated, and the aq layer was extracted with EtOAc (2×). The combined organic extracts were washed with satd NaHCO₃, H₂O, and brine, dried (Na₂SO₄), filtered, and concentrated by rotary evaporation. Purification by silica flash chromatography (7:3 hexanes/EtOAc) afforded the protected N-acyl dipeptide β-ketoester 17a or 17b.

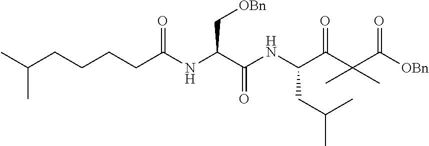 [SMILES: CC(C)CCCCC(=O)N[C@@H](COCc1ccccc1)C(=O)N[C@@H](CC(C)C)C(=O)C(C)(C)C(=O)OCc1ccccc1]

Benzyl (4S)-4-[(2S)-3-(benzyloxy)-2-(6-methylheptanamido)propanamido]-2,2,6-trimethyl-3-oxoheptanoate (17a). 120 mg (70%), colorless oil. TLC: R_{f} 0.4 (2:1 hexanes/EtOAc). IR (ZnSe) 3356.73; 2922.39; 2831.61; 1735.61; 1640.03; 1450.81; 1396.78; 1113.21; 1023.74 ¹H NMR (600 MHz, CDCl₃) δ 7.30-7.18 (m, 10H), 6.84 (d, J=9.0 Hz, 1H), 6.37 (dd, J=12.5, 6.8 Hz, 1H), 5.13-4.98 (m, 2H), 4.95-4.87 (m, 1H), 4.51-4.43 (m, 1H), 4.46 (s, 2H), 3.72 (dd, J=9.2, 4.2 Hz, 1H), 3.31 (dd, J=9.2, 7.7 Hz, 1H), 2.13 (td, J=7.4, 1.8 Hz, 2H), 1.56-1.49 (m, 2H), 1.51-1.39 (m, 2H), 1.39-1.31 (m, 6H), 1.33-1.16 (m, 4H), 1.13-1.06 (m, 2H), 0.78 (dd, J=6.6, 1.5 Hz, 9H), 0.72 (d, J=6.7 Hz, 3H). ¹³C NMR (151 MHz, CDCl₃) δ 207.44, 175.53, 173.46, 172.77, 169.80, 169.54, 137.26, 135.37, 128.63, 128.53, 128.50, 128.39, 128.11, 128.09, 128.02, 127.99, 127.84, 73.58, 73.56, 69.40, 69.32, 67.29, 67.25, 55.08, 55.00, 52.66, 52.64, 52.30, 52.02, 41.56, 41.35, 38.60, 36.53, 27.79, 27.01, 25.80, 24.68, 24.58, 23.41, 22.59, 22.56, 22.27, 22.21, 22.18, 21.15, 21.13, 20.74. HRMS (ESI) m/z calcd for C₃₅H₅₁N₂O₆ ([M+H]⁺) 595.3747. found 595.3755.

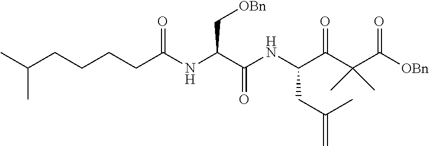 [SMILES: C=C(C)C[C@H](NC(=O)[C@H](COCc1ccccc1)NC(=O)CCCCC(C)C)C(=O)C(C)(C)C(=O)OCc1ccccc1]

Benzyl (S)-4-((S)-3-(benzyloxy)-2-(6-methylheptanamido)propanamido)-2,2,6-trimethyl-3-oxohept-6-enoate (17b). 300 mg (82%), colorless oil. TLC: R_{f} 0.6 (2:1 hexanes/EtOAc). IR (ZnSe) 3290.57; 3067.05; 3033.65; 2952.95; 2931.05; 2867.32; 1743.40; 1716.25; 1640.62; 1541.00; 1498.49; 1455.00; 1386.15; 1365.99; 1250.29; 1214.91; 1142.46; 1107.59; 1029.41; 991.27; 957.78; 897.45; 734.95; 696.61 ¹H NMR (500 MHz, CDCl₃) δ 7.44-7.19 (m, 10H), 6.94 (d, J=8.5 Hz, 1H), 6.42 (d, J=6.7 Hz, 1H), 5.22-5.11 (m, 2H), 5.10-4.99 (m, 1H), 4.77-4.67 (m, 1H), 4.60-4.46 (m, 4H), 3.82-3.71 (m, 1H), 3.45 (dd, J=9.4, 7.6 Hz, 1H), 2.40 (dd, J=14.4, 3.9 Hz, 1H), 2.27-2.16 (m, 2H), 1.65-1.58 (m, 5H), 1.58-1.50 (m, 1H), 1.48 (d, J=2.7 Hz, 3H), 1.43 (d, J=3.0 Hz, 3H), 1.29 (dddd, J=14.1, 11.4, 8.8, 5.7 Hz, 3H), 1.21-1.15 (m, 2H), 0.87 (dd, J=7.1, 2.7 Hz, 6H). ¹³C NMR (126 MHz, CDCl₃) δ 206.74, 174.08, 173.36, 172.82, 169.75, 140.17, 137.35, 135.36, 128.62, 128.50, 128.46, 128.41, 128.13, 127.96, 127.94, 127.90, 127.81, 127.65, 114.45, 73.47, 73.25, 69.77, 69.20, 67.30, 60.39, 55.11, 52.48, 52.38, 51.91, 40.40, 38.63, 38.60, 36.50, 27.81, 27.79, 27.00, 26.98, 25.81, 25.77, 22.59, 22.34, 22.29, 22.18, 21.66, 21.03, 20.69, 14.20, 14.13. HRMS (ESI) m/z calcd for C₃₅H₄₉N₂O₆ ([M+H]⁺) 593.3591. found 593.3578.

### Global Deprotection of β-Ketoester by Pd-Catalyzed Hydrogenolysis (1Ja)

General Protocol. In a round bottom flask, protected β-ketoester 17a (1 equiv) was dissolved in MeOH (0.1 M). Pd/C (1:1 w/w) was added and the mixture was stirred under H2 at atmospheric pressure at rt for 2 h until complete conversion as determined by LC-MS analysis. The mixture was filtered through a 0.2 μm syringe filter to remove Pd/C and concentrated by rotary evaporation. The crude product was dissolved in 50:50 CH₃CN/water. Purification by HPLC (45-95% CH₃CN in H2O, 0.1% TFA) afforded the free β-ketoacid 11a.

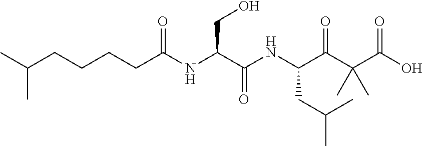 [SMILES: CC(C)CCCCC(=O)N[C@@H](CO)C(=O)N[C@@H](CC(C)C)C(=O)C(C)(C)C(=O)O]

(4S)-4-[(2S)-3-Hydroxy-2-(6-methylheptanamido)propanamido]-2,2,6-trimethyl-3-oxoheptanoic acid (11a). 59 mg (28%), white powder. TLC: R_{f} 0.57 (9:1 CH₂Cl₂/MeOH). IR (ZnSe) 3320.09; 2945.01; 2832.77; 1645.04; 1538.25; 1449.67; 1416.65; 1115.40; 1023.19 ¹H NMR (500 MHz, CDCl₃) δ 7.77 (d, J=8.0 Hz, 1H), 7.58 (d, J=9.4 Hz, 1H), 5.04 (td, J=9.7, 4.1 Hz, 1H), 4.76 (dt, J=8.5, 4.3 Hz, 1H), 3.99-3.89 (m, 1H), 3.84-3.70 (m, 1H), 2.34 (t, J=7.7 Hz, 2H), 1.68-1.46 (m, 5H), 1.44 (s, 3H), 1.40 (s, 3H), 1.36-1.23 (m, 3H), 1.23-1.13 (m, 3H), 1.00-0.86 (m, 6H), 0.86 (dd, J=6.4, 4.5 Hz, 7H). ¹³C NMR (126 MHz, CDCl₃) δ 207.81, 175.49, 174.61, 170.50, 62.63, 58.14, 58.06, 57.98, 54.67, 54.23, 54.21, 53.24, 53.15, 41.14, 38.57, 36.25, 27.77, 26.97, 25.83, 24.63, 23.33, 23.30, 22.49, 22.27, 22.19, 21.76, 21.68, 21.28, 21.25, 18.20, 18.17, 18.14, 18.09, 18.06, 18.03. HRMS (ESI) m/z calcd for C₂₁H₃₈N₂O₆ ([M+Na]⁺) 437.2628. found 437.2620.

Global Deprotection of β-Ketoester with Boron Trichloride (11b).

General Protocol. In a roundbottom flask, protected β-ketoester 17b (1 equiv) was dissolved in anhyd CH₂Cl₂ (0.1 M) and cooled to −78° C. Boron trichloride (6 equiv) was added dropwise and after 20 min, full conversion was detected via TLC analysis. The solvent was removed by rotary evaporation and the mixture was dissolved in CH₃CN/water (1:1). Purification by HPLC (5-65% CH₃CN in H₂O with 0.1% TFA, 20 min) afforded the free β-ketoacid 11b.

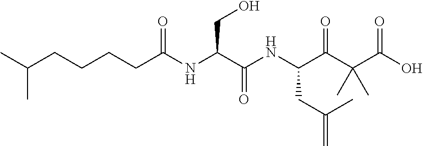 [SMILES: C=C(C)C[C@H](NC(=O)[C@H](CO)NC(=O)CCCCC(C)C)C(=O)C(C)(C)C(=O)O]

(S)-4-((S)-3-Hydroxy-2-(6-methylheptanamido)propanamido)-2,2,6-trimethyl-3-oxohept-6-enoic acid (11b). 50 mg (24%), white powder. TLC: R_{f} 0.53 (9:1 CH₂Cl₂/MeOH). IR (ZnSe)¹H NMR (500 MHz, CDCl₃) δ 7.44-7.18 (m, 2H), 5.09 (td, J=9.8, 4.1 Hz, 1H), 4.87-4.74 (m, 2H), 4.72-4.54 (m, 2H), 3.99 (ddd, J=34.4, 11.6, 3.9 Hz, 1H), 3.78-3.58 (m, 1H), 2.57 (ddd, J=29.7, 14.3, 4.5 Hz, 1H), 2.33-2.21 (m, 3H), 1.75 (d, J=19.3 Hz, 3H), 1.65-1.51 (m, 3H), 1.42 (d, J=6.8 Hz, 4H), 1.30 (dddd, J=24.1, 16.8, 13.1, 8.2 Hz, 3H), 1.19 (dd, J=7.8, 3.5 Hz, 3H), 0.87 (dd, J=6.6, 2.1 Hz, 6H). 13C NMR (126 MHz, CDCl₃) δ 212.74, 206.56, 176.12, 174.74, 174.11, 170.80, 170.54, 140.56, 140.16, 114.35, 63.00, 54.54, 54.33, 53.85, 53.43, 40.44, 38.58, 36.46, 36.27, 27.83, 27.02, 25.80, 22.57, 22.54, 21.90. HRMS (ESI) m/z calcd for C₂₁H₃₆N₂O₆ ([M+Na]⁺) 435.2471. found 435.2473.

Synthesis of α-Hydroxymethyl-α-methyl-β-ketoacid Substrates (11c, 11d). Compounds 11c and 11d were prepared according to below scheme.

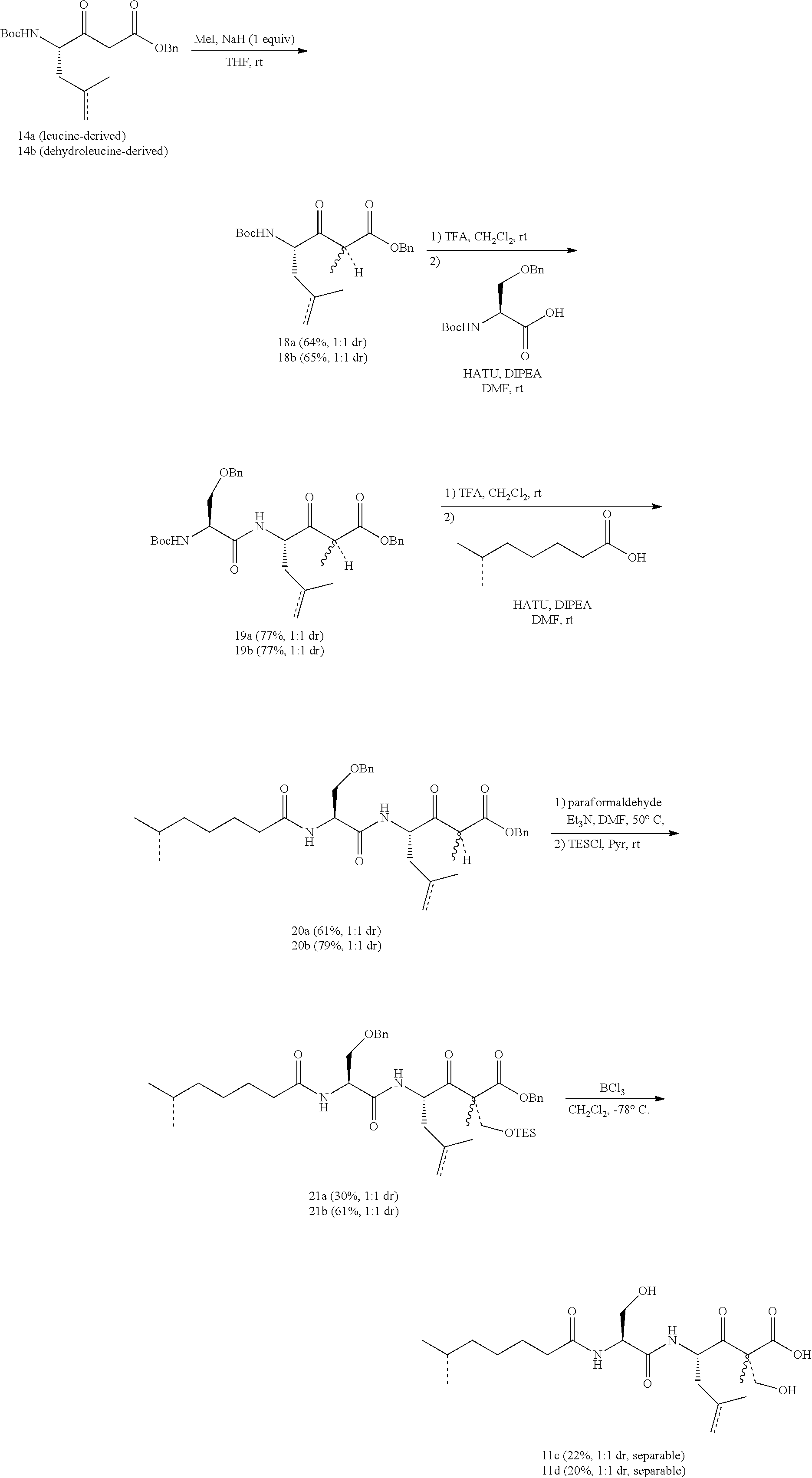

### Monomethylation of β-Ketoesters (18a, 18b)

General Protocol. In a roundbottom flask, β-ketoester 14a or 14b (1 equiv) and iodomethane (9 equiv) were suspended in acetone (0.1 M). Sodium hydride (60% w/w, 1.01 equiv) was washed 5× with pentane, dried in vacuo and resuspended in acetone then added dropwise to the reaction. The mixture was stirred at rt overnight until complete conversion as determined by LC-MS analysis. The mixture was diluted in H₂O and the aq layer was extracted with EtOAc (3×). The combined organic extracts were washed with brine, dried (Na₂SO₄), filtered, and concentrated by rotary evaporation. Purification by silica flash chromatography (9:1 hexanes/EtOAc) afforded α-monomethyl-β-ketoester 18a or 18b.

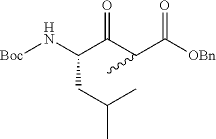 [SMILES: CC(C)C[C@H](NC(=O)OC(C)(C)C)C(=O)C(C)C(=O)OCc1ccccc1]

Benzyl (4S)-4-((tert-butoxycarbonyl)amino)-2,6-dimethyl-3-oxoheptanoate (18a). 4.2 g (64%), colorless oil. TLC: R_{f} 0.84 (2:1 hexanes/EtOAc). IR (ZnSe) 3378.90; 2959.18; 2871.87; 1748.12; 1709.23; 1499.58; 1455.22; 1367.23; 1318.19; 1249.52; 1166.85; 1116.80; 1081.91; 1025.91; 951.39; 912.04; 780.82; 738.75; 698.07 ¹H NMR (500 MHz, CDCl₃) δ 7.33-7.21 (m, 5H), 5.17-5.01 (m, 2H), 4.80-4.66 (m, 1H), 4.39-4.29 (m, 1H), 3.74 (p, J=6.9 Hz, 1H), 1.67-1.43 (m, 1H), 1.35 (d, J=2.8 Hz, 10H), 1.30-1.14 (m, 3H), 0.92-0.84 (m, 1H), 0.84-0.79 (m, 6H). ¹³C NMR (126 MHz, CDCl₃) δ 206.06, 205.96, 170.06, 169.92, 155.49, 135.41, 135.35, 128.67, 128.59, 128.51, 128.47, 128.39, 128.27, 79.96, 67.20, 67.08, 57.87, 57.21, 49.96, 49.09, 40.48, 39.68, 28.38, 28.29, 24.80, 23.30, 23.25, 21.44, 13.31, 12.79. HRMS (ESI) m/z calcd for C₂₁H₃₁NO₅ Na ([M+Na]⁺) 400.2100. found 400.2098.

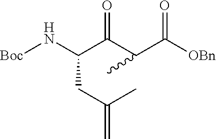 [SMILES: C=C(C)C[C@H](NC(=O)OC(C)(C)C)C(=O)C(C)C(=O)OCc1ccccc1]

Benzyl (4S)-4-((tert-butoxycarbonyl)amino)-2,6-dimethyl-3-oxohept-6-enoate (18b). 2.9 g (65%), colorless oil. TLC: R_{f} 0.81 (2:1 hexanes/EtOAc). IR (ZnSe) 3384.66; 3072.21; 3034.70; 2979.02; 2939.91; 2255.01; 1747.18; 1709.92; 1650.69; 1497.99; 1455.11; 1367.28; 1318.65; 1247.91; 1167.72; 1080.63; 1053.30; 1012.42; 951.91; 906.95; 858.26 ¹H NMR (500 MHz, CDCl₃) δ 7.33-7.22 (m, 5H), 5.17-5.00 (m, 2H), 4.74 (d, J=12.1 Hz, 2H), 4.67-4.51 (m, 1H), 4.39 (td, J=6.6, 4.1 Hz, 1H), 3.84-3.75 (m, 1H), 2.51-2.40 (m, 1H), 2.14-2.05 (m, 1H), 1.64-1.58 (m, 2H), 1.35 (d, J=4.6 Hz, 11H), 1.29 (t, J=7.1 Hz, 2H) ¹³C NMR (126 MHz, CDCl₃) δ 205.52, 205.25, 170.14, 170.02, 155.53, 155.41, 140.85, 140.60, 135.39, 128.69, 128.61, 128.59, 128.54, 128.45, 128.42, 128.30, 114.49, 114.36, 80.11, 80.08, 67.24, 67.10, 57.51, 56.64, 50.19, 49.22, 39.86, 38.74, 28.27, 28.25, 21.78, 21.72, 13.22, 12.67. HRMS (ESI) m/z calcd for C₂₁H₂₉NO₅ Na ([M+Na]⁺) 398.1943. found 398.1940.

Boc Deprotection of β-Ketoesters and Coupling with Protected Serine (19a, 19b).

General Protocol. In a roundbottom flask, Boc-protected β-ketoester 18a or 18b (1 equiv) was dissolved in anhyd CH₂Cl₂ and trifluoroacetic acid (4:1) and the reaction was stirred until complete conversion as determined by LC-MS analysis. The solvent was removed by rotary evaporation and the crude amine was carried forward directly to the next step without further purification.

In a glass vial, the crude amine was dissolved in DMF (0.2 M) and cooled to 0° C. O-Benzyl-N-(tert-butoxycarbonyl)-L-serine (1.1 equiv) and HATU (1.1 equiv) were suspended in anhyd DMF (0.2 M). The crude amine solution in DMF was added dropwise to the stirring solution of HATU and O-benzyl-N-(tert-butoxycarbonyl)-L-serine (0.1 M final conc) and stirred for 10 min. N,N-Diisopropylethylamine (3 equiv) was added dropwise. The reaction was allowed to warm to rt and stirred until complete conversion as determined by LC-MS analysis. The mixture was diluted with EtOAc and H₂O, the layers were separated, and the aq layer was extracted with EtOAc (2×). The combined organic extracts were washed with satd NaHCO₃, H₂O, and brine, dried (Na₂SO₄), filtered, and concentrated by rotary evaporation. Purification by silica flash chromatography (7:3 hexanes/EtOAc) afforded the dipeptide β-ketoester 19a or 19b.

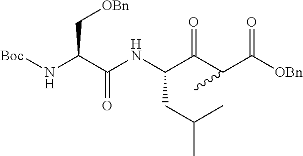 [SMILES: CC(C)C[C@H](NC(=O)[C@H](COCc1ccccc1)NC(=O)OC(C)(C)C)C(=O)C(C)C(=O)OCc1ccccc1]

Benzyl (4S)-4-((S)-3-(benzyloxy)-2-((tert-butoxycarbonyl)amino)propanamido)-2,6-dimethyl-3-oxoheptanoate (19a). 4.75 g (77%), colorless oil. TLC: R_{f} 0.58 (2:1 hexanes/EtOAc). IR (ZnSe) 3418.75; 3338.33; 2958.88; 2870.79; 2253.18; 1747.17; 1715.01; 1674.60; 1496.68; 1454.56; 1367.38; 1323.60; 1238.88; 1165.29; 1111.42; 1081.52; 1044.98; 1025.50; 907.21; 730.12; 697.67; 648.35 ¹H NMR (500 MHz, CDCl₃) δ 7.35-7.10 (m, 10H), 7.02-6.74 (m, 1H), 5.29 (s, 1H), 5.07 (d, J=6.7 Hz, 2H), 4.81-4.54 (m, 1H), 4.46 (d, J=8.0 Hz, 2H), 4.21 (s, 1H), 3.93-3.64 (m, 2H), 3.58-3.36 (m, 1H), 1.51-1.44 (m, 2H), 1.37 (s, 9H), 1.29 (d, J=7.3 Hz, 2H), 1.25 (d, J=6.7 Hz, 2H), 0.87-0.59 (m, 6H). ¹³C NMR (126 MHz, CDCl₃) δ 204.69, 170.51, 170.37, 169.82, 137.32, 135.38, 135.22, 128.65, 128.60, 128.50, 128.47, 128.40, 128.26, 127.99, 127.96, 127.89, 127.85, 80.34, 73.61, 73.57, 69.86, 67.43, 67.16, 56.48, 56.11, 49.92, 49.09, 40.48, 39.31, 28.28, 24.77, 24.72, 23.26, 21.34, 21.31, 13.33, 12.52. HRMS (ESI) m/z calcd for C₃₁H₄₂N₂O₇ Na ([M+Na]⁺) 577.2890. found 577.2864.

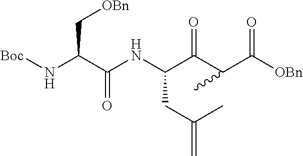 [SMILES: C=C(C)C[C@H](NC(=O)[C@H](COCc1ccccc1)NC(=O)OC(C)(C)C)C(=O)C(C)C(=O)OCc1ccccc1]

Benzyl (4S)-4-((S)-3-(benzyloxy)-2-((tert-butoxycarbonyl)amino)propanamido)-2,6-dimethyl-3-oxohept-6-enoate (19b). 3.3 g (77%), colorless oil. TLC: R_{f} 0.46 (2:1 hexanes/EtOAc). IR (ZnSe) 3326.39; 2977.75; 2928.94; 2870.58; 2101.59; 1747.17; 1715.76; 1673.85; 1497.27; 1454.31; 1366.67; 1238.54; 1216.43; 1165.79; 1105.59; 1085.99; 1046.46; 1025.25; 951.20; 905.01; 861.53 ¹H NMR (500 MHz, CDCl₃) δ 7.41-7.27 (m, 10H), 7.11-6.81 (m, 1H), 5.38 (s, 1H), 5.16 (d, J=5.2 Hz, 2H), 4.88-4.82 (m, 1H), 4.77-4.66 (m, 2H), 4.55 (tt, J=11.5, 6.7 Hz, 3H), 4.30 (s, 1H), 3.95-3.84 (m, 2H), 3.57 (ddd, J=9.2, 6.8, 4.3 Hz, 1H), 2.53 (ddd, J=13.4, 7.8, 4.7 Hz, 1H), 2.24-2.12 (m, 1H), 1.66 (s, 1H), 1.62 (s, 1H), 1.46 (d, J=2.9 Hz, 9H), 1.40 (d, J=7.1 Hz, 1H), 1.34 (d, J=7.0 Hz, 1H), 1.28 (s, 1H). 13C NMR (126 MHz, CDCl₃) δ 204.44, 204.09, 170.50, 170.38, 169.88, 169.68, 155.54, 140.51, 140.20, 137.38, 137.34, 135.36, 135.22, 128.67, 128.61, 128.53, 128.47, 128.43, 128.30, 127.98, 127.95, 127.91, 127.85, 114.54, 114.48, 80.27, 73.53, 73.47, 69.80, 69.73, 67.44, 67.17, 56.18, 55.68, 53.86, 53.50, 50.19, 49.29, 39.68, 38.33, 29.70, 29.66, 28.29, 21.73, 13.22, 12.43. HRMS (ESI) m/z calcd for C₃₁H₄₀N₂O₇ Na ([M+Na]⁺) 575.2733. found 575.2721.

### Boc Deprotection of Dipeptides and N-Acylation (20a, 20b).

General Protocol. In a roundbottom flask, Boc-protected dipeptide O-ketoester 19a or 19b (1 equiv) was dissolved in a mixture of anhyd CH₂Cl₂ and trifluoroacetic acid (4:1) and the reaction was stirred until complete conversion as determined by LC-MS analysis. The solvent was removed by rotary evaporation and the crude amine was carried forward directly to the next step without further purification.

In a glass vial, the crude amine was dissolved in DMF (0.2 M) and cooled at 0° C. 6-Methylheptanoic acid (1.1 equiv) and HATU (1.1 equiv) were suspended in anhyd DMF (0.2 M). The crude amine solution in DMF was added dropwise to the stirring solution of HATU and 6-methylheptanoic acid (0.1 M final conc) and stirred for 10 min. N,N-Diisopropylethylamine (3 equiv) was added dropwise. The reaction was allowed to warm to rt and stirred until complete conversion as determined by LC-MS analysis. The mixture was diluted with EtOAc and H₂O, the layers were separated, and the aq layer was extracted with EtOAc (2×). The combined organic extracts were washed with satd NaHCO₃, H₂O, and brine, dried (Na₂SO₄), filtered, and concentrated by rotary evaporation. Purification by silica flash chromatography (7:3 hexanes/EtOAc) afforded the protected N-acyl dipeptide β-ketoester 20a or 20b.

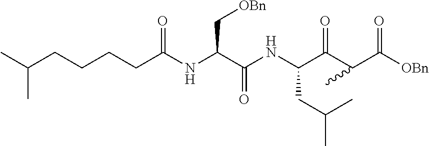 [SMILES: CC(C)CCCCC(=O)N[C@@H](COCc1ccccc1)C(=O)N[C@@H](CC(C)C)C(=O)C(C)C(=O)OCc1ccccc1]

Benzyl (4S)-4-((S)-3-(benzyloxy)-2-(6-methylheptanamido)propanamido)-2,6-dimethyl-3-oxoheptanoate (20a). 330 mg (61%), colorless oil. TLC: R_{f} 0.53 (2:1 hexanes/EtOAc). IR (ZnSe) 3301.36; 2953.33; 1752.40; 1642.26; 1548.35; 1455.39; 1385.93; 1340.41; 1242.88; 1203.57; 1190.34; 1114.86; 1080.85; 1029.31 ¹H NMR (500 MHz, CDCl₃) δ 7.45-7.20 (m, 10H), 7.15 (dd, J=27.6, 8.2 Hz, 1H), 6.48 (dd, J=13.5, 6.7 Hz, 1H), 5.20-5.08 (m, 2H), 4.73-4.51 (m, 3H), 3.92-3.77 (m, 2H), 3.53 (t, J=8.4 Hz, 1H), 2.23 (td, J=7.6, 2.6 Hz, 2H), 1.67-1.42 (m, 5H), 1.41-1.23 (m, 7H), 1.18 (q, J=7.5 Hz, 2H), 1.01-0.72 (m, 12H). ¹³C NMR (126 MHz, CDCl₃) δ 204.88, 204.69, 173.40, 173.35, 170.54, 170.37, 169.82, 156.78, 138.33, 137.33, 137.29, 135.93, 135.37, 135.24, 128.64, 128.59, 128.51, 128.49, 128.44, 128.40, 128.35, 128.30, 128.25, 128.06, 128.02, 128.00, 127.96, 127.93, 127.91, 127.86, 127.79, 127.57, 77.30, 73.60, 73.56, 72.98, 69.49, 68.45, 67.39, 67.16, 66.55, 66.41, 60.39, 56.67, 56.37, 52.24, 51.96, 49.86, 49.05, 41.12, 40.23, 39.10, 38.61, 38.48, 36.54, 34.14, 29.51, 28.93, 27.80, 27.02, 25.81, 25.33, 24.86, 24.82, 24.72, 23.21, 22.59, 22.32, 22.18, 21.35, 21.29, 13.37, 12.54. HRMS (ESI) m/z calcd for C₃₄H₄₇N₂O₇ ([M+H]⁺) 581.3591. found 581.3591.

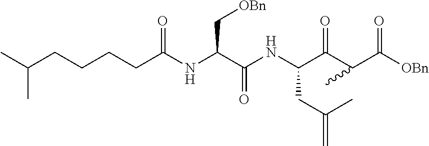 [SMILES: C=C(C)C[C@H](NC(=O)[C@H](COCc1ccccc1)NC(=O)CCCCC(C)C)C(=O)C(C)C(=O)OCc1ccccc1]

Benzyl (4S)-4-((S)-3-(benzyloxy)-2-(6-methylheptanamido)propanamido)-2,6-dimethyl-3-oxohept-6-enoate (20b). 167 mg (79%), colorless oil. TLC: R_{f} 0.59 (2:1 hexanes/EtOAc). IR (ZnSe) 3319.20; 2944.25; 2832.43; 1640.79; 1453.21; 1115.57; 1023.49 ¹H NMR (500 MHz, CDCl₃) δ 7.42-7.25 (m, 10H), 6.99 (d, J=7.2 Hz, 1H), 6.35 (t, J=7.1 Hz, 1H), 5.17 (d, J=6.8 Hz, 2H), 4.79-4.65 (m, 2H), 4.57 (qd, J=13.1, 6.7 Hz, 4H), 3.93-3.82 (m, 2H), 3.52 (q, J=7.6 Hz, 1H), 2.52 (ddd, J=14.9, 11.1, 4.7 Hz, 1H), 2.22 (dd, J=9.5, 6.0 Hz, 3H), 1.72-1.58 (m, 5H), 1.48-1.27 (m, 6H), 1.19 (q, J=7.3 Hz, 2H), 0.88 (dd, J=6.6, 2.2 Hz, 6H). ¹³C NMR (126 MHz, CDCl₃) δ 204.22, 204.05, 173.35, 173.30, 170.39, 170.25, 169.85, 169.77, 140.43, 140.08, 137.31, 137.29, 135.33, 135.22, 128.66, 128.62, 128.51, 128.48, 128.45, 128.42, 128.40, 128.30, 128.14, 128.04, 128.01, 127.98, 127.94, 127.81, 127.77, 127.54, 114.63, 114.52, 73.59, 73.54, 69.32, 69.29, 67.45, 67.20, 56.23, 55.79, 52.11, 51.78, 50.25, 49.41, 39.58, 38.60, 38.32, 36.55, 27.79, 27.01, 25.78, 22.59, 21.67, 21.61, 13.28, 12.41. HRMS (ESI) m/z calcd for C₃₄H₄₆N₂O₆ Na ([M+Na]⁺) 601.3254. found 601.3282.

### α-Hydroxymethylation of β-Ketoesters (21a, 21b).

General Protocol. In a roundbottom flask, α-monomethyl-β-ketoester 20a or 20b (1 equiv) and triethylamine (3 equiv) were suspended in DMF (0.1 M). Paraformaldehyde (20 equiv) was added to the mixture and stirred at 50° C. for 12 h until complete conversion as determined by TLC analysis. The mixture was diluted in water and extracted with EtOAc (3×). The combined organic extracts were washed with brine, dried (Na₂SO₄), filtered, and concentrated by rotary evaporation. The crude alcohol was carried forward directly to the next step without further purification.

In a roundbottom flask, the crude alcohol was dissolved in pyridine (0.1 M) and TESCl (10 equiv) was added dropwise. The reaction was stirred for 16 h until complete conversion as determined by TLC analysis. The mixture was diluted with water and EtOAc. The aq layer was extracted with EtOAc (3×). The combined organic extracts were washed with CuSO₄ (3×) and brine, dried (Na₂SO₄), filtered, and concentrated by rotary evaporation. Purification by silica flash chromatography (7:3 hexanes/EtOAc) afforded the protected α-hydroxymethyl-α-methyl-β-ketoester 21a or 21b.

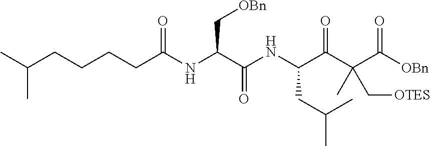

Benzyl (4S)-4-((S)-3-(benzyloxy)-2-(6-methylheptanamido)propanamido)-2,6-dimethyl-3-oxo-2-(((triethylsilyl)oxy)methyl)heptanoate (21a). 80 mg (30% over two steps), colorless oil. TLC: R_{f} 0.87 (2:1 hexanes/EtOAc). IR (ZnSe) 3343.07; 2950.84; 2834.40; 1644.02; 1450.69; 1413.50; 1107.47; 1023.55 ¹H NMR (500 MHz, CDCl₃) δ 7.25 (q, J=8.4, 6.9 Hz, 10H), 6.88 (dd, J=46.1, 8.9 Hz, 1H), 6.18 (d, J=6.4 Hz, 1H), 5.13-5.00 (m, 2H), 4.98-4.88 (m, 1H), 4.47 (s, 2H), 4.43 (tt, J=7.3, 3.4 Hz, 1H), 3.96 (dd, J=9.7, 3.1 Hz, 1H), 3.91 (dd, J=9.9, 2.1 Hz, 1H), 3.79-3.71 (m, 1H), 3.28 (td, J=8.6, 5.8 Hz, 1H), 2.17-2.07 (m, J=7.3 Hz, 2H), 1.51 (h, J=7.6 Hz, 52H), 1.49-1.32 (m, 6H), 1.22 (dd, J=13.9, 7.2 Hz, 3H), 1.11 (ddd, J=15.4, 10.3, 5.0 Hz, 2H), 0.81 (dt, J=22.5, 7.3 Hz, 19H), 0.72 (dd, J=11.6, 6.4 Hz, 3H), 0.49 (q, J=7.9 Hz, 5H). ¹³C NMR (126 MHz, CDCl₃) δ 206.66, 205.96, 173.19, 170.79, 170.64, 169.69, 169.56, 137.31, 135.46, 135.40, 128.55, 128.52, 128.49, 128.29, 128.24, 128.13, 128.11, 128.00, 127.95, 73.60, 73.56, 69.33, 67.20, 66.07, 65.81, 61.24, 61.09, 60.40, 53.63, 53.14, 51.94, 51.89, 41.07, 40.79, 38.61, 36.60, 27.80, 27.03, 25.78, 24.81, 24.68, 23.43, 22.58, 21.20, 21.15, 21.05, 17.91, 17.61, 14.21, 6.68, 4.23, 4.20. HRMS (ESI) m/z calcd for C₄₁H₆₅N₂O₇Si ([M+H]⁺) 725.4561. found 725.4552.

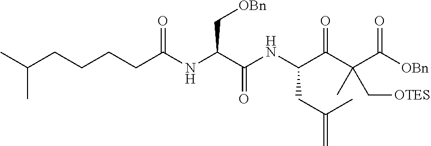

Benzyl (4S)-4-((S)-3-(benzyloxy)-2-(6-methylheptanamido)propanamido)-2,6-dimethyl-3-oxo-2-(((triethylsilyl)oxy)methyl)hept-6-enoate (21b). 220 mg (61% over two steps), colorless oil. TLC: R_{f} 0.87 (2:1 hexanes/EtOAc). IR (ZnSe) 3328.42; 2950.01; 2833.77; 1746.00; 1645.82; 1453.59; 1413.25; 1107.05; 1023.05 ¹H NMR (500 MHz, CDCl₃) δ 7.24 (p, J=7.3, 6.2 Hz, 10H), 6.83 (dd, J=41.0, 8.3 Hz, 1H), 6.16 (d, J=6.4 Hz, 1H), 5.15-5.01 (m, 2H), 4.97 (ddt, J=10.8, 8.5, 3.1 Hz, 1H), 4.62 (d, J=5.2 Hz, 1H), 4.52-4.35 (m, 3H), 4.09-3.98 (m, 1H), 3.97-3.86 (m, 1H), 3.73 (ddt, J=9.3, 7.3, 3.6 Hz, 1H), 3.33 (dq, J=16.4, 8.7, 8.2 Hz, 1H), 2.38 (ddd, J=18.4, 14.4, 3.6 Hz, 1H), 2.11 (td, J=7.4, 2.7 Hz, 2H), 2.04-1.84 (m, 2H), 1.61-1.48 (m, 4H), 1.43 (s, 2H), 1.20 (dt, J=18.4, 7.2 Hz, 5H), 1.09 (h, J=7.6 Hz, 2H), 0.93-0.80 (m, 8H), 0.78 (d, J=6.7 Hz, 7H), 0.59-0.42 (m, 6H). ¹³C NMR (126 MHz, CDCl₃) δ 206.19, 205.35, 173.20, 173.18, 171.13, 170.90, 170.73, 169.69, 169.56, 140.35, 140.33, 137.41, 137.36, 135.45, 135.39, 128.55, 128.52, 128.46, 128.32, 128.26, 128.21, 128.16, 128.13, 128.11, 128.00, 127.96, 127.93, 127.80, 114.34, 114.28, 73.52, 73.48, 69.13, 67.22, 66.22, 65.84, 61.35, 61.18, 60.39, 53.32, 52.80, 51.76, 51.73, 40.01, 39.80, 38.61, 36.58, 34.13, 33.91, 29.51, 27.79, 27.02, 25.75, 22.58, 21.67, 21.63, 21.04, 17.84, 17.79, 14.20, 6.67, 4.30, 4.22, 4.20, 4.17, 3.99. HRMS (ESI) m/z calcd for C₄₁H₆₃N₂O₇Si ([M+H]⁺) 723.4405. found 723.4406.

Global Deprotection of β-Ketoesters with Boron Trichloride (11c, 11d).

General Protocol. In a roundbottom flask, protected β-ketoester 21a or 21b (1 equiv) was dissolved in anhyd CH₂Cl₂ (0.1 M) and cooled to −78° C. Boron trichloride (6 equiv) was added dropwise and the reaction was stirred for 20 min until complete conversion as determined by TLC analysis. The solvent was removed by rotary evaporation and the crude product was dissolved in CH₃CN/water (1:1). Purification by HPLC (5-65% CH₃CN in H₂O with 0.1% TFA, 20 min) afforded the individual diastereomers (relative stereochemistry not determined) of free β-ketoacid 11c or 11d.

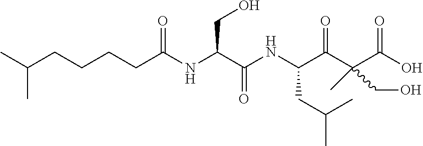 [SMILES: CC(C)CCCCC(=O)N[C@@H](CO)C(=O)N[C@@H](CC(C)C)C(=O)C(C)(CO)C(=O)O]

### (4S)-4-((S)-3-Hydroxy-2-(6-methylheptanamido)propanamido)-2-(hydroxymethyl)-2,6-dimethyl-3-oxoheptanoic Acid (11c)

Diastereomer 1: 6 mg (13%), white powder. TLC: R_{f} 0.5 (9:1 CH₂Cl₂/MeOH). IR (ZnSe) 3306.74; 3084.26; 2955.55; 2931.54; 2870.35; 1737.84; 1642.79; 1540.31; 1466.33; 1367.27; 1228.88; 1216.79; 1143.98; 1055.00; 910.02; 734.82 ¹H NMR (500 MHz, CDCl₃) δ 7.70 (d, J=9.0 Hz, 1H), 6.63 (dd, J=61.9, 7.7 Hz, 1H), 5.16 (t, J=9.1 Hz, 1H), 4.63 (s, 1H), 4.10-4.01 (m, 1H), 3.90 (d, J=12.2 Hz, 1H), 3.65-3.53 (m, 1H), 2.28 (q, J=9.3, 7.6 Hz, 3H), 1.93 (s, 1H), 1.63 (h, J=7.5 Hz, 2H), 1.58-1.42 (m, 3H), 1.41-1.25 (m, 5H), 1.20 (p, J=7.8, 6.8 Hz, 3H), 1.06-0.98 (m, 4H), 0.98-0.92 (m, 3H), 0.88 (dd, J=6.6, 1.9 Hz, 7H). HRMS (ESI) m/z calcd for C₂₁H₃₈N₂O₇ ([M+Na]⁺) 453.2577. found 453.2577.

Diastereomer 2: 4.4 mg (9%), white powder. TLC: R_{f} 0.5 (9:1 CH₂Cl₂/MeOH). IR (ZnSe) 3291.63; 2955.83; 2869.92; 2252.49; 1723.02; 1643.11; 1539.75; 1467.56; 1384.46; 1368.14; 1230.41; 1205.30; 1172.24; 1138.99; 1063.26; 909.84; 734.83 ¹H NMR (500 MHz, CDCl₃) δ 7.07 (d, J=62.9 Hz, 1H), 6.49 (d, J=17.2 Hz, 1H), 4.61 (d, J=77.6 Hz, 2H), 4.09 (d, J=11.9 Hz, 1H), 3.90-3.53 (m, 2H), 3.06 (s, OH), 2.25 (s, 2H), 1.63 (s, 6H), 1.55 (p, J=7.1, 6.5 Hz, 1H), 1.44 (d, J=12.2 Hz, 1H), 1.20 (d, J=7.6 Hz, 4H), 1.06-0.91 (m, 9H), 0.88 (dt, J=7.0, 2.0 Hz, 7H).

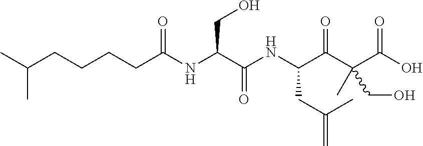 [SMILES: C=C(C)C[C@H](NC(=O)[C@H](CO)NC(=O)CCCCC(C)C)C(=O)C(C)(CO)C(=O)O]

### (4S)-4-((S)-3-Hydroxy-2-(6-methylheptanamido)propanamido)-2-(hydroxymethyl)-2,6-dimethyl-3-oxohept-6-enoic Acid (11d)

Diastereomer 1: 11 mg (13%), white powder. TLC: R_{f} 0.53 (9:1 CH₂Cl₂/MeOH). IR (ZnSe) 3298.30; 3078.89; 2954.11; 2933.40; 2869.99; 2253.65; 1708.25; 1537.10; 1461.94; 1383.27; 1287.61; 1244.19; 1206.51; 1180.98; 1145.76; 1051.73; 906.43; 732.48 ¹H NMR (500 MHz, CDCl₃) δ 7.55 (d, J=8.7 Hz, 1H), 6.79-6.59 (m, 1H), 5.43-5.15 (m, 1H), 4.91-4.80 (m, 2H), 4.77 (d, J=17.1 Hz, 1H), 4.57 (d, J=26.1 Hz, 1H), 4.09-4.00 (m, 1H), 3.98-3.85 (m, 1H), 3.85-3.71 (m, 1H), 3.61 (q, J=10.2, 7.9 Hz, 1H), 2.62-2.50 (m, 1H), 2.36-2.28 (m, 1H), 2.28-2.17 (m, 3H), 1.78 (d, J=8.0 Hz, 2H), 1.75 (d, J=6.1 Hz, 2H), 1.62 (p, J=7.8 Hz, 2H), 1.57-1.51 (m, 1H), 1.50 (s, 1H), 1.43-1.24 (m, 4H), 1.19 (q, J=7.0, 5.1 Hz, 2H), 0.89 (d, J=1.4 Hz, 3H), 0.88 (d, J=1.5 Hz, 3H). HRMS (ESI) m/z calcd for C₂₁H₃₆N₂O₇ ([M+Na]⁺) 451.2420. found 451.2430.

Diastereomer 2: 6 mg (7%), white powder. TLC: R_{f} 0.53 (9:1 CH₂Cl₂/MeOH). (1:1 hexanes/EtOAc). IR (ZnSe) 3302.69; 3080.15; 2953.93; 2933.31; 2869.78; 2252.71; 2225.53; 1712.41; 1644.91; 1532.63; 1462.82; 1383.35; 1367.62; 1287.18; 1244.64; 1203.79; 1179.96; 1142.48; 1053.90; 908.66; 734.60 ¹H NMR (500 MHz, CDCl₃) δ 7.05 (d, J=6.1 Hz, 1H), 6.49 (dd, J=17.0, 7.3 Hz, 1H), 4.90 (d, J=43.9 Hz, 2H), 4.83-4.68 (m, 1H), 4.55 (s, 1H), 4.07 (d, J=7.3 Hz, 1H), 3.80-3.57 (m, 2H), 3.09 (d, J=7.5 Hz, 1H), 2.54 (dd, J=14.3, 4.6 Hz, 1H), 2.23 (qd, J=14.5, 13.2, 7.3 Hz, 3H), 1.94 (s, 1H), 1.79-1.72 (m, 3H), 1.66-1.49 (m, 4H), 1.33 (dq, J=15.6, 7.3 Hz, 3H), 1.20 (q, J=7.4 Hz, 3H), 1.04 (d, J=6.8 Hz, 2H), 0.88 (d, J=6.6 Hz, 6H).

Synthesis of Other β-Ketoacid Substrate Analogues (11f-11r). Compounds 11f-11r were prepared according to below scheme.

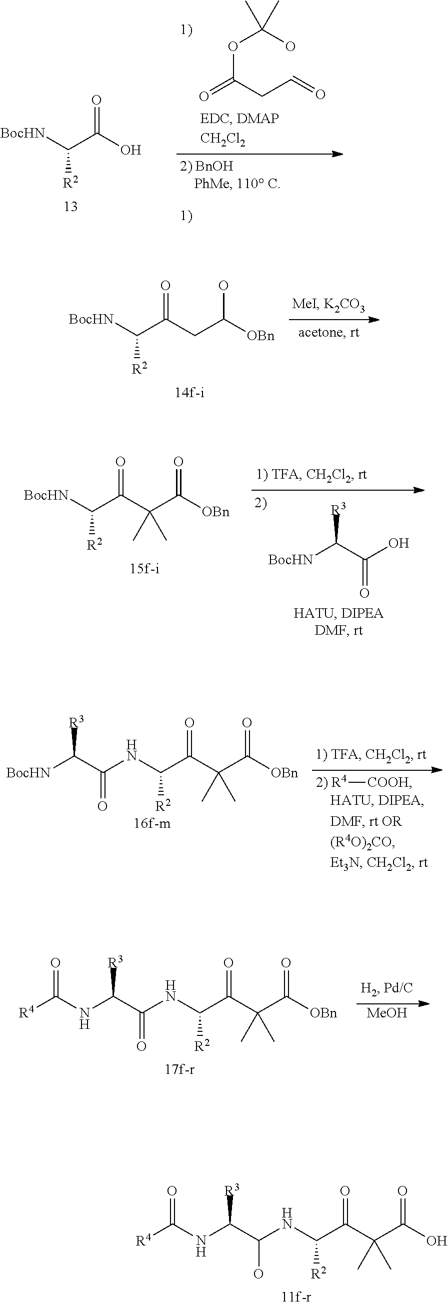

Synthesis of β-Ketoester Fragments (14f-14i).

General Protocol. In a roundbottom flask, the appropriate Boc-protected amino acid 13 (1 equiv), DMAP (1.1 equiv), and Meldrum's acid (1.1 equiv) were suspended in anhyd CH₂Cl₂ (0.1 M), then EDC (3 equiv) was added portion-wise and the reaction was stirred until complete conversion as determined by TLC analysis. The mixture was poured over 1 N HCl and extracted with CH₂Cl₂ (2×). The combined organic extracts were washed with brine, dried (NaSO₄), filtered, and concentrated by rotary evaporation. The crude Meldrum's acid adduct and benzyl alcohol (1.1 equiv) were dissolved in anhyd toluene (0.1 M) and heated to 110° C. for 4 h, then concentrated by rotary evaporation. Purification by silica flash chromatography (9:1 hexanes/EtOAc) afforded the O-benzyl p-ketoester 14.

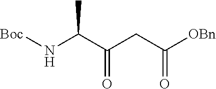 [SMILES: C[C@H](NC(=O)OC(C)(C)C)C(=O)CC(=O)OCc1ccccc1]

Benzyl (4S)-4-{[(tert-butoxy)carbonyl]amino}-3-oxopentanoate (14f). 469 mg (55% over two steps), white crystalline solid. TLC: R_{f} 0.7 (2:1 Hexanes:EtOAC). IR (ZnSe) 3032.52; 2979.45; 2933.82; 1745.90; 1497.99; 1454.41; 1367.72; 1316.67; 1264.83; 1249.52; 1046.43; 1022.95. ¹H NMR (500 MHz, CDCl₃) δ 7.31-7.23 (m, 5H), 5.25 (d, J=7.2 Hz, 1H), 5.08 (d, J=2.9 Hz, 2H), 4.23 (p, J=7.0 Hz, 1H), 3.56-3.44 (m, 2H), 1.36 (s, 9H), 1.20 (d, J=7.2 Hz, 3H). ¹³C NMR (126 MHz, CDCl₃) δ 202.55, 166.96, 155.37, 141.19, 135.31, 128.71-127.87 (m), 127.44, 126.97, 80.14, 67.26 (d, J=11.0 Hz), 64.96, 55.46 (d, J=9.7 Hz), 45.72, 28.32 (d, J=9.2 Hz), 16.76 (d, J=7.2 Hz). HRMS (ESI) m/z calcd for C₁₇H₂₃NO₅ ([M+H]⁺) 344.1474. found 344.1485.

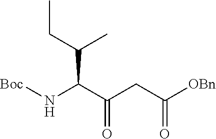 [SMILES: CCC(C)[C@H](NC(=O)OC(C)(C)C)C(=O)CC(=O)OCc1ccccc1]

Benzyl (4S)-4-{[(tert-butoxy)carbonyl]amino}-5-methyl-3-oxoheptanoate (14g). 789 mg (96% over two steps), white crystalline powder. TLC: R_{f} 0.17 (9:1 hexanes/EtOAc). IR (ZnSe) 3364.04; 2970.42; 2933.79; 2878.69; 1747.38; 1499.44; 1456.30; 1367.42; 1314.41; 1265.80; 1165.58. ¹H NMR (500 MHz, CDCl₃) δ 7.39-7.30 (m, 7H), 5.30-5.21 (m, 1H), 5.21-5.10 (m, 2H), 4.35-3.97 (m, 1H), 3.65-3.53 (m, 2H), 2.00-1.77 (m, 1H), 1.45 (s, 9H), 1.36-1.23 (m, 1H), 1.17-1.00 (m, 1H), 1.00-0.81 (m, 6H). ¹³C NMR (126 MHz, CDCl₃) δ 202.33, 166.71, 155.89, 141.25, 135.35, 128.61, 128.58, 128.43, 128.41, 128.37, 128.26, 127.39, 126.92, 89.85, 81.00, 80.01, 67.15, 66.02, 64.94, 64.33, 58.25, 47.16, 46.35, 37.02, 36.13, 28.37, 28.30, 28.16, 24.73, 24.38, 24.09, 15.99, 15.68, 11.56, 11.43, 11.29. HRMS (ESI) m/z calcd for C₂₀H₂₉NO₅ ([M+Na]⁺) 386.1946. found 386.1943.

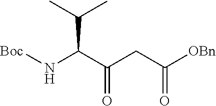 [SMILES: CC(C)[C@H](NC(=O)OC(C)(C)C)C(=O)CC(=O)OCc1ccccc1]

Benzyl (4S)-4-{[(tert-butoxy)carbonyl]amino}-5-methyl-3-oxohexanoate (14h). 494 mg (62% over two steps), white crystalline solid. TLC: R_{f} 0.17 (9:1 hexanes/EtOAc). IR (ZnSe) 2968.76; 2931.50; 1746.13; 1708.33; 1498.13; 1455.14; 1391.92; 1367.16; 1311.74; 1244.07; 1161.01; 1011.45. ¹H NMR (500 MHz, CDCl₃) δ 7.41-7.29 (m, 5H), 5.26 (d, J=8.9 Hz, 1H), 5.20-5.10 (m, 2H), 4.15 (ddd, J=161.8, 9.1, 5.5 Hz, 1H), 3.58 (s, 2H), 2.26-2.01 (m, 1H), 1.45 (s, 9H), 1.03-0.94 (m, 3H), 0.90-0.73 (m, 3H). ¹³C NMR (126 MHz, CDCl₃) δ 202.13, 166.69, 155.94, 141.26, 135.33, 128.59, 128.42, 128.37, 128.26, 127.38, 126.92, 89.67, 79.98, 67.16, 66.43, 66.02, 64.92, 64.44, 59.08, 46.97, 46.27, 30.60, 29.40, 28.36, 28.30, 28.15, 19.74, 19.40, 17.92, 17.23, 16.73. HRMS (ESI) m/z calcd for C₁₉H₂₇NO₅ ([M+Na]⁺) 372.1793. found 372.1787.

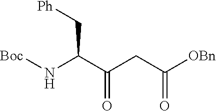 [SMILES: CC(C)(C)OC(=O)N[C@@H](Cc1ccccc1)C(=O)CC(=O)OCc1ccccc1]

Benzyl (4S)-4-{[(tert-butoxy)carbonyl]amino}-3-oxo-5-phenylpentanoate (14i). 556 mg (74% over two steps), white crystalline solid. TLC: R_{f} 0.17 (9:1 hexanes/EtOAc). IR (ZnSe) 3419.46; 3031.38; 2979.07; 2932.64; 2874.28; 1744.28; 1496.81; 1454.57; 1392.65; 1368.15; 1317.10; 1251.89; 1164.37; 1044.60; 1021.7. ¹H NMR (500 MHz, CDCl₃) δ 7.45-7.08 (m, 10H), 5.19 (q, J=10.0, 7.3 Hz, 2H), 5.07 (d, J=8.2 Hz, 1H), 4.62-4.29 (m, 1H), 3.64-3.45 (m, 2H), 3.20-3.02 (m, 1H), 3.06-2.82 (m, 1H), 1.42 (s, 9H). 13C NMR (126 MHz, CDCl₃) δ 201.87, 166.77, 155.29, 136.15, 135.31, 129.29, 128.72, 128.65, 128.63, 128.49, 128.41, 128.14, 127.05, 80.26, 67.20, 62.30, 60.56, 46.84, 46.20, 36.86, 28.27. HRMS (ESI) m/z calcd for C₂₃H₂₇NO₅ ([M+Na]⁺) 420.1791. found 420.1787.

Dimethylation of β-Ketoesters (15f-15i)

General Protocol. In a roundbottom flask, β-ketoester 14 (1 equiv) and K₂CO₃ (2.1 equiv) were suspended in anhyd acetone (0.1 M). Iodomethane (9 equiv) was added dropwise via syringe and the reaction was stirred at rt until complete conversion as determined by TLC analysis. Water was added to the mixture and the aq layer was extracted with EtOAc (3×). The combined organic extracts were washed with brine, dried (Na₂SO₄), filtered, and concentrated by rotary evaporation. Purification by silica flash chromatography (9:1 hexanes/EtOAc) afforded the α,α-dimethyl-β-ketoester 15.

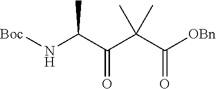 [SMILES: C[C@H](NC(=O)OC(C)(C)C)C(=O)C(C)(C)C(=O)OCc1ccccc1]

Benzyl 4-{[(tert-butoxy)carbonyl]amino}-2,2-dimethyl-3-oxopentanoate (15f). 106 mg (21%), colorless oil. TLC: R_{f} 0.25 (9:1 hexanes/EtOAc). IR (ZnSe) 3382.20; 2980.74; 2936.08; 1709.35; 1499.20; 1455.58; 1391.47; 1367.72; 1247.66; 1162.86; 1029.03; 997.00; 909.82; 861.28; 755.21. ¹H NMR (500 MHz, CDCl₃) δ 7.40-7.30 (m, 5H), 5.22-5.12 (m, 2H), 4.94 (d, J=9.0 Hz, 1H), 4.66 (p, J=7.6 Hz, 1H), 1.46 (s, 3H), 1.45-1.41 (m, 12H), 1.20 (d, J=7.2 Hz, 3H). ¹³C NMR (126 MHz, CDCl₃) δ 208.29, 172.79, 154.71, 135.32, 128.64, 128.42, 128.20, 79.83, 67.14, 54.50, 51.54, 28.34, 28.28, 22.39, 22.33, 22.15, 22.09, 18.81, 18.76. HRMS (ESI) m/z calcd for C17H23NO5 ([M+H]⁺) 344.1474. found 344.1485.

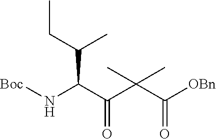 [SMILES: CCC(C)[C@H](NC(=O)OC(C)(C)C)C(=O)C(C)(C)C(=O)OCc1ccccc1]

Benzyl (4S)-4-{[(tert-butoxy)carbonyl]amino}-2,2,5-trimethyl-3-oxoheptanoate (15g). 284 mg (35%), colorless oil. TLC: R_{f} 0.4 (9:1 hexanes/EtOAc). IR (ZnSe) 3382.66; 2969.75; 2934.73; 2877.66; 1706.24; 1497.73; 1456.77; 1390.97; 1366.88; 1257.66; 1235.49; 1043.33; 1006.30. ¹H NMR (500 MHz, CDCl₃) δ 7.37-7.26 (m, 6H), 5.22 (d, J=12.3 Hz, 1H), 5.07 (d, J=12.3 Hz, 1H), 4.77 (d, J=10.3 Hz, 1H), 4.54 (dd, J=10.3, 4.7 Hz, 1H), 1.92-1.80 (m, 1H), 1.45-1.39 (m, 16H), 1.32-1.20 (m, 1H), 0.84 (d, J=6.8 Hz, 3H), 0.79 (d, 3H). ¹³C NMR (126 MHz, CDCl₃) δ 207.60, 172.76, 155.43, 135.44, 128.60, 128.35, 128.22, 79.77, 66.98, 60.38, 54.69, 36.11, 28.29, 28.24, 23.14, 22.74, 22.68, 22.05, 22.00, 15.94, 11.55. HRMS (ESI) m/z calcd for C₂₂H₃₃NO₅ ([M+Na]⁺) 414.2271. found 414.2256.

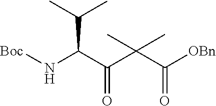 [SMILES: CC(C)[C@H](NC(=O)OC(C)(C)C)C(=O)C(C)(C)C(=O)OCc1ccccc1]

Benzyl (4S)-4-{[(tert-butoxy)carbonyl]amino}-2,2,5-trimethyl-3-oxohexanoate (15h). 385 mg (72%), colorless oil. TLC: R_{f} 0.4 (9:1 hexanes/EtOAc). IR (ZnSe) 3378.81; 2973.82; 2934.98; 2875.61; 1705.80; 1497.14; 1456.87; 1390.88; 1367.15; 1238.82; 1157.87; 1142.22; 1043.18; 1002.19; 877.04. ¹H NMR (500 MHz, CDCl₃) δ 7.35-7.30 (m, 5H), 5.18 (d, J=12.3 Hz, 1H), 5.08 (d, J=12.5 Hz, 1H), 4.84 (d, J=10.5 Hz, 1H), 4.52 (dd, J=10.2, 4.4 Hz, 1H), 2.11 (dp, J=17.5, 6.0 Hz, 1H), 1.40 (d, J=8.6 Hz, 16H), 0.84 (d, J=6.6 Hz, 3H), 0.66 (d, J=6.7 Hz, 3H). ¹³C NMR (126 MHz, CDCl₃) δ 207.37, 172.71, 155.46, 135.43, 128.58, 128.34, 128.24, 79.71, 67.07, 66.99, 66.91, 61.39, 60.24, 54.64, 29.43, 28.28, 28.22, 22.65, 22.58, 22.07, 22.02, 19.79, 16.04. HRMS (ESI) m/z calcd for C₂₁H₃₁NO₅ ([M+Na]⁺) 400.2096. found 400.2100.

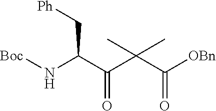 [SMILES: CC(C)(C)OC(=O)N[C@@H](Cc1ccccc1)C(=O)C(C)(C)C(=O)OCc1ccccc1]

Benzyl (4S)-4-{[(tert-butoxy)carbonyl]amino}-2,2-dimethyl-3-oxo-5-phenylpentanoate (15i). 515 mg (45%), colorless oil. TLC: R_{f} 0.35 (9:1 hexanes/EtOAc). IR (ZnSe) 3377.49; 2979.66; 2933.82; 1497.06; 1455.28; 1391.17; 1367.03; 1247.12; 1216.14; 1149.49; 1023.16; 908.72; 751.09. ¹H NMR (500 MHz, CDCl₃) δ 7.28-7.24 (m, 4H), 7.18-7.08 (m, 3H), 7.00 (d, J=7.3 Hz, 2H), 5.09 (d, J=11.5 Hz, 1H), 5.02 (d, J=12.3 Hz, 1H), 4.82-4.74 (m, 1H), 4.69 (d, J=9.9 Hz, 1H), 3.03 (dd, J=14.0, 5.3 Hz, 1H), 2.62 (dd, J=14.0, 8.4 Hz, 1H), 1.38-1.09 (m, 15H). ¹³C NMR (126 MHz, CDCl₃) δ 207.40, 172.81, 154.79, 136.56, 135.49, 129.49, 128.70, 128.43, 128.39, 128.28, 126.70, 80.30, 79.84, 67.13, 67.04, 58.27, 56.66, 54.76, 39.04, 38.29, 30.87, 28.22, 28.16, 22.29, 22.23, 21.73, 21.68. HRMS (ESI) m/z calcd for C₂₃H₃₁NO₅ ([M+H]⁺) 426.2270. found 426.2280.

Boc Deprotection of β-Ketoesters and Coupling with Protected Amino Acids (16f-16m).

General Protocol. In a roundbottom flask, Boc-protected β-ketoester 15 (1 equiv) was dissolved in a mixture of anhyd CH₂Cl₂ and trifluoroacetic acid (4:1) and the reaction was stirred until complete conversion as determined by LC-MS analysis. The solvent was removed by rotary evaporation and the crude amine was carried forward directly to the next step without further purification.

In a glass vial, the crude amine was dissolved in DMF (0.2 M) and cooled at 0° C. The appropriate Boc-protected amino acid (1.1 equiv) and HATU (1.1 equiv) were suspended in anhyd DMF (0.2 M). The crude amine solution in DMF was added dropwise to the stirring solution of HATU and Boc-protected amino acid (0.1 M final conc) and stirred for 10 min. N,N-Diisopropylethylamine (3 equiv) was added dropwise. The reaction was allowed to warm to rt and stirred until complete conversion as determined by LC-MS analysis. The mixture was diluted with EtOAc and H₂O, the layers were separated, and the aq layer was extracted with EtOAc (2×). The combined organic extracts were washed with satd NaHCO₃, H₂O, and brine, dried (Na₂SO₄), filtered, and concentrated by rotary evaporation. Purification by silica flash chromatography (7:3 hexanes/EtOAc) afforded the dipeptide β-ketoester 16.

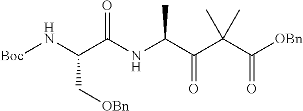 [SMILES: C[C@H](NC(=O)[C@H](COCc1ccccc1)NC(=O)OC(C)(C)C)C(=O)C(C)(C)C(=O)OCc1ccccc1]

Benzyl (4S)-4-[(2S)-3-(benzyloxy)-2-{[(tert-butoxy)carbonyl]amino}propanamido]-2,2-dimethyl-3-oxopentanoate (16f). 74 mg (92%), colorless oil. TLC: R_{f} 0.6 (2:1 hexanes:EtOAc). IR (ZnSe) 3321.76; 3032.83; 2980.45; 2935.76; 2870.46; 1789.86; 1716.16; 1672.30; 1498.60; 1455.03; 1391.42; 1367.23; 1253.32; 1164.30; 1113.57; 1027.40. ¹H NMR (500 MHz, CDCl₃) δ 7.31-7.20 (m, 10H), 6.93 (s, 1H), 5.30-5.25 (m, 1H), 5.13-5.03 (m, 2H), 4.94-4.84 (m, 1H), 4.49-4.41 (m, 2H), 4.14 (s, 1H), 3.77 (dd, J=9.4, 3.9 Hz, 1H), 3.42 (dd, J=9.3, 6.3 Hz, 1H), 1.38 (s, 3H), 1.36 (s, 9H), 1.34 (s, 3H), 1.12 (d, J=6.9 Hz, 3H). ¹³C NMR (126 MHz, CDCl₃) δ 207.39, 172.67, 169.42, 155.43, 137.40, 135.28, 128.63, 128.47, 128.43, 128.25, 127.93, 127.83, 80.29, 73.48, 69.75, 67.43, 67.34, 54.81, 53.99, 50.30, 28.30, 28.24, 22.34, 18.65, 18.60. HRMS (ESI) m/z calcd for C₂₉H₃₈N₂O₇ ([M+Na]⁺) 549.2559. found 549.2577.

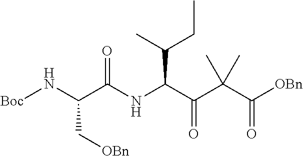 [SMILES: CCC(C)[C@H](NC(=O)[C@H](COCc1ccccc1)NC(=O)OC(C)(C)C)C(=O)C(C)(C)C(=O)OCc1ccccc1]

Benzyl (4S)-4-[(2S)-3-(benzyloxy)-2-{[(tert-butoxy)carbonyl]amino}propanamido]-2,2,5-trimethyl-3-oxoheptanoate (16g). 133 mg (64%), colorless oil. TLC: R_{f} 0.7 (2:1 hexanes:EtOAc). IR (ZnSe) 3427.76; 3338.27; 3034.07; 2971.35; 2934.98; 2876.79; 2252.44; 1710.23; 1676.68; 1496.16; 1455.50; 1391.48; 1367.39; 1256.42; 1158.37; 907.47. ¹H NMR (500 MHz, CDCl₃) δ 7.30-7.16 (m, 11H), 6.87-6.82 (m, 1H), 5.28 (s, 1H), 5.07 (s, 2H), 4.85 (dd, J=9.8, 5.0 Hz, 1H), 4.45 (s, 2H), 4.16 (s, 1H), 3.81 (dd, J=9.3, 3.9 Hz, 1H), 3.46 (dd, J=9.2, 6.5 Hz, 1H), 1.78-1.69 (m, 1H), 1.37 (s, 3H), 1.36 (s, 9H), 1.31 (s, 3H), 1.24-1.13 (m, 1H), 0.73 (d, J=6.8 Hz, 3H), 0.63 (t, J=7.3 Hz, 3H). ¹³C NMR (126 MHz, CDCl₃) δ 206.95, 172.69, 170.13, 155.52, 137.31, 135.45, 128.57, 128.45, 128.32, 128.21, 127.95, 127.94, 80.32, 73.62, 73.60, 69.63, 67.25, 58.56, 55.27, 53.92, 36.38, 28.30, 28.24, 23.06, 22.59, 22.53, 22.39, 22.32, 16.10, 11.38. HRMS (ESI) m/z calcd for C₃₂H₄₄N₂O₇ ([M+H]⁺) 569.3251. found 569.3227.

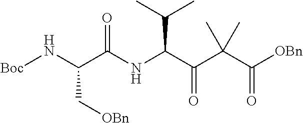 [SMILES: CC(C)[C@H](NC(=O)[C@H](COCc1ccccc1)NC(=O)OC(C)(C)C)C(=O)C(C)(C)C(=O)OCc1ccccc1]

Benzyl (4S)-4-[(2S)-3-(benzyloxy)-2-{[(tert-butoxy)carbonyl]amino}propanamido]-2,2,5-trimethyl-3-oxohexanoate (16h). 194 mg (68%), colorless oil. TLC: R_{f} 0.7 (2:1 hexanes:EtOAc). IR (ZnSe) 3400.57; 3324.28; 3033.54; 2975.76; 2934.15; 2873.77; 2251.70; 1711.43; 1677.92; 1497.33; 1470.24; 1455.20; 1391.29; 1366.94; 1250.48; 1160.95; 1110.80; 1046.08; 1028.03; 910.05. ¹H NMR (500 MHz, CDCl₃) δ 7.29-7.17 (m, 11H), 6.88 (d, J=9.4 Hz, 1H), 5.29 (s, 1H), 5.12-5.02 (m, 2H), 4.84 (dd, J=9.9, 4.5 Hz, 1H), 4.45 (s, 2H), 4.18 (d, J=7.7 Hz, 1H), 3.79 (dd, J=9.2, 4.0 Hz, 1H), 3.45 (dd, J=9.3, 6.4 Hz, 1H), 1.99 (qt, J=7.7, 3.9 Hz, 1H), 1.36 (d, J=2.8 Hz, 12H), 1.32 (s, 3H), 0.74 (d, J=6.8 Hz, 3H), 0.59 (d, J=6.8 Hz, 3H). ¹³C NMR (126 MHz, CDCl₃) δ 206.72, 172.65, 170.17, 155.52, 137.32, 135.44, 128.57, 128.54, 128.44, 128.41, 128.34, 128.27, 128.17, 127.96, 127.93, 127.78, 127.58, 80.28, 73.58, 69.60, 67.34, 67.25, 67.14, 58.51, 55.17, 53.99, 29.86, 28.31, 28.25, 22.51, 22.45, 22.35, 21.04, 20.01, 19.98, 16.17, 14.18. HRMS (ESI) m/z calcd for C₃₁H₄₂N₂O₇ ([M+H]⁺) 555.3060. found 555.3070.

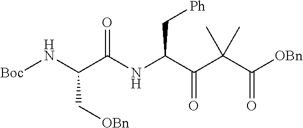 [SMILES: CC(C)(C)OC(=O)N[C@@H](COCc1ccccc1)C(=O)N[C@@H](Cc1ccccc1)C(=O)C(C)(C)C(=O)OCc1ccccc1]

Benzyl (4S)-4-[(2S)-3-(benzyloxy)-2-{[(tert-butoxy)carbonyl]amino}propanamido]-2,2-dimethyl-3-oxo-5-phenylpentanoate (16i). 233 mg (64%), colorless oil. TLC: R_{f} 0.7 (2:1 hexanes:EtOAc). IR (ZnSe) 3414.01; 3325.20; 3064.97; 3031.96; 2980.01; 2932.61; 2868.06; 2251.63; 1712.70; 1677.01; 1496.28; 1454.67; 1391.13; 1366.55; 1248.97; 1161.21; 1103.76; 1049.00; 1026.69; 909.42. ¹H NMR (500 MHz, CDCl₃) δ 7.30-7.16 (m, 12H), 7.15-7.03 (m, 3H), 6.96-6.89 (m, 2H), 6.76 (d, J=9.1 Hz, 1H), 5.16-5.08 (m, 2H), 5.08-4.97 (m, 2H), 4.44-4.31 (m, 2H), 3.62 (s, 1H), 3.33 (dd, J=9.3, 6.7 Hz, 1H), 2.95 (dd, J=14.0, 5.5 Hz, 1H), 2.62 (dd, J=14.0, 8.2 Hz, 1H), 1.34 (s, 9H), 1.27 (d, J=14.9 Hz, 6H). ¹³C NMR (126 MHz, CDCl₃) δ 206.75, 172.67, 169.51, 155.38, 137.37, 136.05, 135.46, 129.39, 128.62, 128.55, 128.50, 128.45, 128.42, 128.38, 128.33, 128.21, 127.97, 127.91, 127.78, 127.59, 126.90, 80.23, 73.46, 73.42, 73.38, 69.52, 67.36, 67.26, 67.18, 60.39, 55.31, 54.67, 53.80, 38.13, 28.31, 28.24, 22.05, 21.98, 21.92, 21.85, 21.05, 14.26. HRMS (ESI) m/z calcd for C₃₅H₄₂N₂O₇ ([M+H]⁺) 603.3092. found 603.3070.

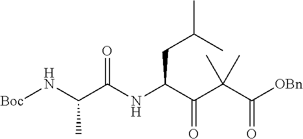 [SMILES: CC(C)C[C@H](NC(=O)[C@H](C)NC(=O)OC(C)(C)C)C(=O)C(C)(C)C(=O)OCc1ccccc1]

Benzyl (4S)-4-[(2S)-2-{[(tert-butoxy)carbonyl]amino}propanamido]-2,2,6-trimethyl-3-oxoheptanoate (16j). 132 mg (83%), colorless oil. TLC: R_{f} 0.7 (2:1 hexanes:EtOAc). IR (ZnSe) 2977.51; 1714.16; 1662.36; 1516.04; 1455.47; 1389.87; 1367.13; 1324.08; 1250.37; 1068.62; 1048.37; 1026.58. ¹H NMR (500 MHz, CDCl₃) δ 7.38-7.27 (m, 5H), 6.46 (d, J=8.5 Hz, 1H), 5.18 (d, J=12.4 Hz, 1H), 5.13 (d, J=12.3 Hz, 1H), 4.96 (t, J=9.9 Hz, 2H), 4.02 (s, 1H), 1.60-1.49 (m, 1H), 1.45-1.35 (m, 17H), 1.25 (d, J=7.0 Hz, 3H), 0.85 (d, J=6.4 Hz, 3H), 0.82 (d, J=6.6 Hz, 3H). ¹³C NMR (126 MHz, CDCl₃) δ 207.90, 172.80, 172.03, 155.53, 135.38, 128.62, 128.39, 128.19, 80.15, 67.34, 67.26, 67.17, 54.88, 52.54, 50.01, 41.54, 28.29, 28.22, 24.55, 23.46, 22.35, 22.29, 22.06, 21.20, 17.51. HRMS (ESI) m/z calcd for C₂₅H₃₈N₂O₆ ([M+H]⁺) 463.2795. found 463.2808.

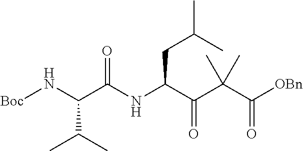 [SMILES: CC(C)C[C@H](NC(=O)[C@@H](NC(=O)OC(C)(C)C)C(C)C)C(=O)C(C)(C)C(=O)OCc1ccccc1]

Benzyl (4S)-4-[(2S)-2-{[(tert-butoxy)carbonyl]amino}-3-methylbutanamido]-2,2,6-trimethyl-3-oxoheptanoate (16k). 85 mg (51%), colorless oil. TLC: R_{f} 0.8 (2:1 hexanes:EtOAc). IR (ZnSe) 3319.43; 2960.90; 1743.58; 1714.10; 1687.19; 1650.17; 1524.71; 1469.71; 1390.49; 1366.85; 1297.51; 1172.05; 1043.35; 1017.95. ¹H NMR (500 MHz, CDCl₃) δ 7.16-7.05 (m, 5H), 5.99 (d, J=9.2 Hz, 1H), 4.96 (d, J=12.3 Hz, 1H), 4.90 (d, J=12.4 Hz, 1H), 4.84-4.72 (m, 2H), 3.57 (dd, J=8.7, 6.4 Hz, 1H), 1.86-1.78 (m, 1H), 1.41-1.30 (m, 1H), 1.23 (s, 3H), 1.20 (s, 9H), 1.18 (s, 3H), 1.17-1.11 (m, 2H), 0.69-0.63 (m, 9H), 0.60 (d, J=6.7 Hz, 3H). ¹³C NMR (126 MHz, CDCl₃) δ 208.05, 172.66, 171.02, 155.81, 135.38, 128.60, 128.38, 128.18, 79.95, 67.26, 60.16, 55.11, 52.13, 41.61, 30.47, 28.29, 28.23, 24.64, 23.47, 22.26, 22.20, 21.16, 19.27, 17.86. HRMS (ESI) m/z calcd for C₂₇H₄₂N₂O₆ ([M+H]⁺) 491.3109. found 491.3121.

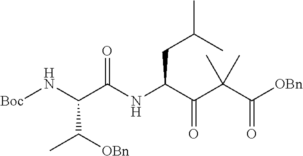 [SMILES: CC(C)C[C@H](NC(=O)[C@@H](NC(=O)OC(C)(C)C)C(C)OCc1ccccc1)C(=O)C(C)(C)C(=O)OCc1ccccc1]

Benzyl (4S)-4-[(2S)-3-(benzyloxy)-2-{[(tert-butoxy)carbonyl]amino}butanamido]-2,2,6-trimethyl-3-oxoheptanoate (161). 209 mg (29%), colorless oil. TLC: R_{f} 0.8 (2:1 hexanes:EtOAc). IR (ZnSe) 3412.70; 3010.58; 2977.54; 2934.99; 2871.60; 1713.49; 1677.36; 1497.69; 1390.29; 1378.49; 1251.27; 1216.48; 1165.07; 1069.75; 752.94. ¹H NMR (500 MHz, CDCl₃) δ 7.38-7.25 (m, 10H), 6.97 (d, J=9.0 Hz, 1H), 5.42 (d, J=7.0 Hz, 1H), 5.19 (d, J=12.4 Hz, 1H), 5.12 (d, J=12.4 Hz, 1H), 4.99 (td, J=9.9, 3.6 Hz, 1H), 4.64 (d, J=11.2 Hz, 1H), 4.52 (d, J=11.2 Hz, 1H), 4.25 (dd, J=7.0, 3.0 Hz, 1H), 4.18-4.12 (m, 1H), 1.46 (d, J=26.1 Hz, OH), 1.52-1.40 (m, 16H), 1.37-1.23 (m, 2H), 1.14 (d, J=6.4 Hz, 3H), 0.89-0.76 (m, 3H), 0.73 (d, J=6.6 Hz, 3H). ¹³C NMR (126 MHz, CDCl₃) δ 207.64, 172.72, 169.50, 155.78, 137.95, 135.44, 128.60, 128.43, 128.35, 128.16, 127.78, 127.71, 80.21, 74.67, 71.41, 67.27, 57.56, 55.19, 52.26, 41.38, 28.31, 24.63, 23.39, 22.36, 22.24, 21.11, 14.97. HRMS (ESI) m/z calcd for C₃₃H₄₈N₂O₇ ([M+H]⁺) 583.3373. found 583.3383.

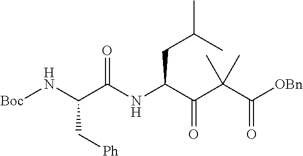 [SMILES: CC(C)C[C@H](NC(=O)[C@H](Cc1ccccc1)NC(=O)OC(C)(C)C)C(=O)C(C)(C)C(=O)OCc1ccccc1]

Benzyl (4S)-4-[(2S)-2-{[(tert-butoxy)carbonyl]amino}-3-phenylpropanamido]-2,2,6-trimethyl-3-oxoheptanoate (16m). 157 mg (85%), colorless oil. TLC: R_{f} 0.9 (2:1 hexanes:EtOAc). IR (ZnSe) 3307.84; 2958.71; 1713.16; 1685.60; 1654.02; 1522.68; 1498.47; 1469.65; 1455.37; 1390.28; 1366.95; 1251.20; 1168.92; 1047.51; 1020.05. ¹H NMR (500 MHz, CDCl₃) δ 7.40-7.20 (m, 8H), 7.20-7.15 (m, 2H), 6.33 (d, J=9.3 Hz, 1H), 5.23-5.11 (m, 2H), 5.04-4.95 (m, 2H), 4.29 (q, J=8.1, 7.6 Hz, 1H), 3.09-2.97 (m, 2H), 1.55-1.47 (m, 1H), 1.42 (s, 12H), 1.36 (t, J=2.6 Hz, 3H), 1.35-1.29 (m, 2H), 0.89 (dd, J=6.7, 4.3 Hz, 4H), 0.81 (d, J=6.7 Hz, 3H). ¹³C NMR (126 MHz, CDCl₃) δ 207.53, 172.71, 170.52, 155.41, 136.49, 135.39, 129.35, 128.63, 128.61, 128.38, 128.18, 128.12, 126.92, 80.27, 67.34, 67.25, 67.17, 60.39, 55.68, 55.02, 52.19, 52.11, 41.61, 37.55, 31.59, 28.25, 28.18, 24.47, 23.43, 22.66, 22.20, 22.14, 21.19, 21.03, 14.12. HRMS (ESI) m/z calcd for C₃₁H₄₂N₂O₆ ([M+H]⁺) 539.3118. found 539.3121.

Boc Deprotection of Dipeptides and N-Acylation with Carboxylic Acids (17f-17m, 17q).

General Protocol. In a roundbottom flask, Boc-protected dipeptide β-ketoester 16 (1 equiv) was dissolved in a mixture of anhyd CH₂Cl₂ and trifluoroacetic acid (4:1) and the reaction was stirred until complete conversion as determined by LC-MS analysis. The solvent was removed by rotary evaporation and the crude amine was carried forward directly to the next step without further purification.

In a glass vial, the crude amine was dissolved in DMF (0.2 M) and cooled at 0° C. The appropriate carboxylic acid (1.1 equiv) and HATU (1.1 equiv) were suspended in anhyd DMF (0.2 M). The crude amine solution in DMF was added dropwise to the stirring solution of HATU and acyl acid (0.1 M final conc) and stirred for 10 min. N,N-Diisopropylethylamine (3 equiv) was added dropwise. The reaction was allowed to warm to rt and stirred until complete conversion as determined by LC-MS analysis. The mixture was diluted with EtOAc and H₂O, the layers were separated, and the aq layer was extracted with EtOAc (2×). The combined organic extracts were washed with satd NaHCO₃, H₂O, and brine, dried (Na₂SO₄), filtered, and concentrated by rotary evaporation. Purification by silica flash chromatography (7:3 hexanes/EtOAc) afforded the N-acyl dipeptide β-ketoester 17.

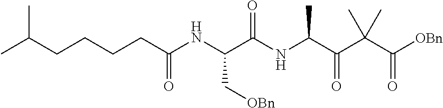 [SMILES: CC(C)CCCCC(=O)N[C@@H](COCc1ccccc1)C(=O)N[C@@H](C)C(=O)C(C)(C)C(=O)OCc1ccccc1]

Benzyl (4S)-4-[(2S)-3-(benzyloxy)-2-(6-methylheptanamido)propanamido]-2,2-dimethyl-3-oxopentanoate (17f). 74 mg (76% over two steps), colorless oil. TLC: R_{f} 0.3 (2:1 hexanes/EtOAc). IR (ZnSe) 3418.88; 3292.57; 3066.73; 3034.12; 2954.65; 2868.37; 2251.06; 1738.74; 1716.76; 1645.55; 1511.60; 1467.66; 1454.96; 1385.99; 1366.87; 1258.71; 1147.87; 1110.29; 1028.34; 1001.43; 908.79. ¹H NMR (500 MHz, CDCl₃) δ 7.26 (ddq, J=10.1, 6.9, 4.2, 3.4 Hz, 11H), 6.93 (d, J=8.1 Hz, 1H), 6.18 (d, J=6.6 Hz, 1H), 5.11-5.03 (m, 2H), 4.91-4.81 (m, 1H), 4.52-4.43 (m, 2H), 4.43-4.38 (m, 1H), 3.75 (dd, J=9.2, 4.1 Hz, 1H), 3.34 (dd, J=9.3, 7.5 Hz, 1H), 2.14 (td, J=7.4, 1.8 Hz, 2H), 1.57-1.41 (m, 4H), 1.38 (s, 3H), 1.35 (s, 3H), 1.26-1.17 (m, 2H), 1.11 (m, 3H), 1.14-1.07 (m, 1H), 0.79 (d, J=6.6 Hz, 6H). ¹³C NMR (126 MHz, CDCl₃) δ 207.24, 206.95, 173.23, 172.71, 169.21, 137.32, 137.26, 135.26, 128.64, 128.53, 128.51, 128.46, 128.22, 128.18, 128.03, 127.99, 127.95, 127.91, 73.55, 67.34, 54.77, 52.22, 52.19, 50.44, 38.61, 36.59, 30.94, 27.80, 27.01, 25.79, 22.59, 22.27, 18.52. HRMS (ESI) m/z calcd for C₃₂H₄₄N₂O₆ ([M+Na]⁺) 575.3105. found 575.3097.

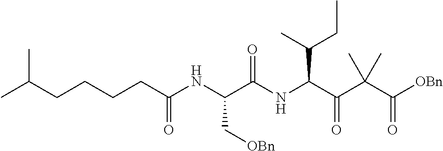 [SMILES: CCC(C)[C@H](NC(=O)[C@H](COCc1ccccc1)NC(=O)CCCCC(C)C)C(=O)C(C)(C)C(=O)OCc1ccccc1]

Benzyl (4S)-4-[(2S)-3-(benzyloxy)-2-(6-methylheptanamido)propanamido]-2,2,5-trimethyl-3-oxoheptanoate (17g). 112 mg (79% over two steps), colorless oil. TLC: R_{f} 0.45 (2:1 hexanes/EtOAc). IR (ZnSe) 3286.10; 3064.91; 3034.17; 2957.96; 2869.28; 1742.27; 1713.32; 1640.44; 1543.11; 1455.85; 1385.50; 1366.21; 1265.06; 1212.94; 1141.88; 1051.39; 1028.78; 969.57; 909.36. ¹H NMR (500 MHz, CDCl₃) δ 7.38-7.28 (m, 10H), 7.00 (d, J=9.4 Hz, 1H), 6.30 (d, J=6.3 Hz, 1H), 5.15 (d, J=2.2 Hz, 2H), 4.90 (ddd, J=9.5, 4.6, 2.0 Hz, 1H), 4.58-4.51 (m, 3H), 3.85 (ddd, J=9.2, 4.3, 1.9 Hz, 1H), 3.49-3.42 (m, 1H), 2.21 (t, J=7.1 Hz, 2H), 1.81 (m, 1H), 1.63-1.56 (m, 2H), 1.56-1.48 (m, 1H), 1.45 (s, 3H), 1.40 (s, 3H), 1.35-1.26 (m, 2H), 1.26-1.13 (m, 3H), 0.86 (m, 7H), 0.83-0.75 (m, 3H), 0.74-0.68 (m, 3H). ¹³C NMR (126 MHz, CDCl₃) δ 206.70, 173.38, 172.79, 170.13, 137.20, 135.41, 128.59, 128.48, 128.36, 128.19, 128.08, 128.01, 73.66, 69.12, 67.27, 59.02, 55.15, 52.11, 38.61, 36.58, 36.15, 27.80, 27.03, 25.82, 23.02, 22.59, 22.45, 22.39, 16.21, 11.48. HRMS (ESI) m/z calcd for C₃₅H₅₀N₂O₆ ([M+Na]⁺) 617.3553. found 617.3567.

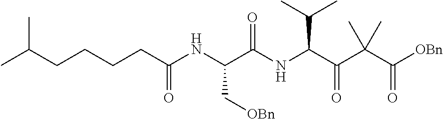 [SMILES: CC(C)CCCCC(=O)N[C@@H](COCc1ccccc1)C(=O)N[C@H](C(=O)C(C)(C)C(=O)OCc1ccccc1)C(C)C]

Benzyl (4S)-4-[(2S)-3-(benzyloxy)-2-(6-methylheptanamido)propanamido]-2,2,5-trimethyl-3-oxohexanoate (17h). 138 mg (66% over two steps), colorless oil. TLC: R_{f} 0.45 (2:1 hexanes/EtOAc). IR (ZnSe) 3283.89; 3062.76; 2956.24; 2869.14; 1739.64; 1713.52; 1642.16; 1539.57; 1467.93; 1389.35; 1368.45; 1265.60; 1214.07; 1141.96; 1114.14; 1044.89. ¹H NMR (500 MHz, CDCl₃) δ 7.35 (dq, J=4.7, 3.2, 2.6 Hz, 9H), 7.05 (d, J=9.5 Hz, 1H), 6.30 (d, J=6.3 Hz, 1H), 5.22-5.12 (m, 2H), 4.96-4.89 (m, 1H), 4.61-4.52 (m, 3H), 3.87 (dd, J=8.7, 4.1 Hz, 1H), 3.50-3.43 (m, 1H), 2.23 (t, J=7.8 Hz, 2H), 1.62 (p, J=7.4 Hz, 2H), 1.54 (dt, J=13.0, 6.7 Hz, 1H), 1.47 (s, 3H), 1.43 (s, 3H), 1.37-1.26 (m, 3H), 1.19 (q, J=7.7 Hz, 2H), 0.90-0.86 (m, 6H), 0.86-0.83 (m, 3H), 0.66 (d, J=6.9 Hz, 3H). ¹³C NMR (126 MHz, CDCl₃) δ 206.52, 173.38, 172.77, 170.13, 137.21, 135.38, 128.59, 128.49, 128.38, 128.26, 128.07, 128.01, 73.66, 67.29, 55.07, 52.17, 36.58, 29.59, 25.80, 22.58, 20.05, 15.99. HRMS (ESI) m/z calcd for C₃₄H₄₈N₂O₆ ([M+Na]⁺) 603.3415. found 603.3410.

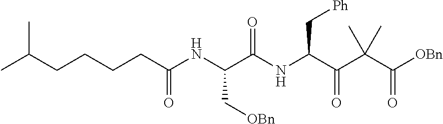 [SMILES: CC(C)CCCCC(=O)N[C@@H](COCc1ccccc1)C(=O)N[C@@H](Cc1ccccc1)C(=O)C(C)(C)C(=O)OCc1ccccc1]

Benzyl (4S)-4-[(2S)-3-(benzyloxy)-2-(6-methylheptanamido)propanamido]-2,2-dimethyl-3-oxo-5-phenylpentanoate (17i). 183 mg (74% over two steps), colorless oil. TLC: R_{f} 0.45 (2:1 hexanes/EtOAc). IR (ZnSe) 3284.75; 3064.65; 3031.80; 2952.67; 2866.57; 1743.45; 1715.66; 1640.52; 1545.20; 1497.42; 1467.38; 1455.03; 1387.31; 1366.15; 1254.46; 1215.31; 1143.75; 1108.69; 1029.10; 1001.16; 967.69; 910.53. ¹H NMR (500 MHz, CDCl₃) δ 7.41-7.26 (m, 11H), 7.25-7.15 (m, 3H), 7.09-6.98 (m, 2H), 6.94 (d, J=9.0 Hz, 1H), 6.15 (d, J=6.5 Hz, 1H), 5.24-5.16 (m, 1H), 5.15 (d, J=1.9 Hz, 2H), 4.54 (d, J=11.7 Hz, 1H), 4.49 (d, J=11.6 Hz, 1H), 4.41 (td, J=7.1, 4.3 Hz, 1H), 3.78-3.72 (m, 1H), 3.42-3.34 (m, 1H), 3.07 (dd, J=14.1, 5.2 Hz, 1H), 2.70 (dd, J=14.1, 8.6 Hz, 1H), 2.16 (td, J=7.4, 2.0 Hz, 2H), 1.61-1.50 (m, 2H), 1.42 (s, 3H), 1.39 (s, 3H), 1.38-1.26 (m, 3H), 1.23-1.15 (m, 2H), 0.88 (d, J=6.7 Hz, 6H). 13C NMR (126 MHz, CDCl₃) δ 206.56, 173.23, 172.79, 169.46, 137.31, 136.04, 135.40, 129.32, 128.65, 128.53, 128.48, 128.42, 128.17, 128.03, 128.00, 126.94, 73.53, 73.50, 73.47, 69.01, 68.94, 67.31, 67.26, 55.22, 55.13, 55.03, 52.01, 51.95, 38.61, 37.99, 36.52, 27.80, 27.01, 25.74, 22.60, 22.24, 22.18, 21.99, 21.93. HRMS (ESI) m/z calcd for C₃₈H₄₈N₂O₆ ([M+Na]⁺) 651.3425. found 651.3410.

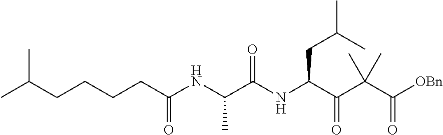 [SMILES: CC(C)CCCCC(=O)N[C@@H](C)C(=O)N[C@@H](CC(C)C)C(=O)C(C)(C)C(=O)OCc1ccccc1]

Benzyl (4S)-2,2,6-trimethyl-4-[(2S)-2-(6-methylheptanamido)propanamido]-3-oxoheptanoate (17j). 102 mg (73% over two steps), colorless oil. TLC: R_{f} 0.6 (2:1 hexanes/EtOAc). IR (ZnSe) 3286.85; 2956.26; 2869.95; 1741.45; 1716.01; 1642.38; 1544.45; 1468.22; 1456.36; 1387.14; 1368.28; 1255.96; 1216.62; 1144.16; 1046.29 ¹H NMR (500 MHz, CDCl₃) δ 7.38-7.26 (m, 5H), 6.63 (d, J=9.1 Hz, 1H), 6.17 (d, J=7.5 Hz, 1H), 5.21-5.08 (m, 2H), 4.93 (ddd, J=10.7, 9.1, 3.3 Hz, 1H), 4.43 (p, J=7.0 Hz, 1H), 2.21-2.09 (m, 2H), 1.66-1.51 (m, 2H), 1.55-1.45 (m, 2H), 1.44 (s, 3H), 1.39 (s, 3H), 1.33-1.27 (m, 2H), 1.27-1.23 (m, 3H), 1.19-1.10 (m, 2H), 0.84 (m, J=6.6 Hz, 9H), 0.82 (d, J=6.5 Hz, 3H). ¹³C NMR (126 MHz, CDCl₃) δ 207.67, 173.02, 172.80, 171.94, 151.72, 135.36, 128.65, 128.46, 128.20, 128.14, 67.24, 54.86, 52.86, 52.81, 48.57, 48.52, 41.28, 38.60, 36.59, 27.78, 27.01, 25.87, 24.64, 23.39, 22.57, 22.41, 22.35, 22.12, 22.06, 21.19, 18.10, 18.05. HRMS (ESI) m/z calcd for C₂₈H₄₄N₂O₅ ([M+Na]⁺) 511.3172. found 511.3148.

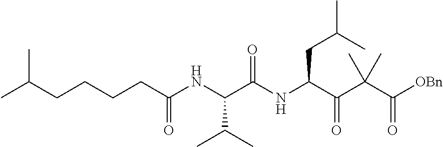 [SMILES: CC(C)CCCCC(=O)N[C@H](C(=O)N[C@@H](CC(C)C)C(=O)C(C)(C)C(=O)OCc1ccccc1)C(C)C]

Benzyl (4S)-2,2,6-trimethyl-4-[(2S)-3-methyl-2-(6-methylheptanamido)butanamido]-3-oxoheptanoate (17k). 72 mg (80% over two steps), colorless oil. TLC: R_{f} 0.7 (2:1 hexanes/EtOAc). IR (ZnSe) 3280.81; 3019.09; 2961.05; 2871.65; 1737.21; 1714.14; 1645.91; 1511.45; 1468.87; 1389.64; 1370.21; 1215.89; 1147.06; 755.93 ¹H NMR (500 MHz, CDCl₃) δ 7.16-7.06 (m, 5H), 5.99 (d, J=9.0 Hz, 1H), 5.82 (d, J=8.7 Hz, 1H), 4.97 (d, J=12.3 Hz, 1H), 4.91 (d, J=12.3 Hz, 1H), 4.77 (ddd, J=10.4, 9.0, 3.5 Hz, 1H), 3.98 (dd, J=8.7, 6.9 Hz, 1H), 1.98 (t, J=7.6 Hz, 2H), 1.77 (dt, J=13.6, 6.8 Hz, 1H), 1.44-1.31 (m, 2H), 1.29 (dd, J=13.2, 6.6 Hz, 1H), 1.24 (s, 3H), 1.19 (s, 3H), 1.19-1.11 (m, 2H), 1.08 (dd, J=7.6, 1.8 Hz, 1H), 1.04 (dt, J=13.5, 5.3 Hz, 1H), 0.98-0.90 (m, 2H), 0.66 (m, J=6.7, 1.3 Hz, 6H), 0.63 (m, J=6.6, 4.9 Hz, 9H), 0.60 (d, J=6.7 Hz, 3H). ¹³C NMR (126 MHz, CDCl₃) δ 207.88, 173.27, 172.64, 170.78, 135.34, 128.64, 128.43, 128.17, 67.28, 58.17, 55.10, 52.39, 52.32, 41.47, 38.61, 36.80, 31.04, 27.82, 27.01, 26.01, 24.72, 23.41, 22.57, 22.29, 21.17, 19.15, 18.11. HRMS (ESI) m/z calcd for C30H48N2O5 ([M+Na]⁺) 539.3477. found 539.3461.

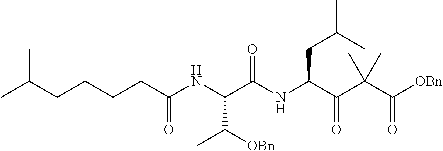 [SMILES: CC(C)CCCCC(=O)N[C@H](C(=O)N[C@@H](CC(C)C)C(=O)C(C)(C)C(=O)OCc1ccccc1)C(C)OCc1ccccc1]

Benzyl (4S)-4-[(2S)-3-(benzyloxy)-2-(6-methylheptanamido)butanamido]-2,2,6-trimethyl-3-oxoheptanoate (171). 43 mg (81% over two steps), colorless oil. TLC: R_{f} 0.7 (2:1 hexanes/EtOAc. IR (ZnSe) 3296.49; 2956.60; 2869.83; 2251.13; 1740.57; 1715.63; 1498.23; 1468.60; 1385.87; 1261.45; 1144.38; 1099.45; 909.12 ¹H NMR (500 MHz, CDCl₃) δ 7.34-7.17 (m, 10H), 6.95 (d, J=8.7 Hz, 1H), 6.33 (d, J=6.3 Hz, 1H), 5.12 (d, J=12.5 Hz, 1H), 5.06 (d, J=12.6 Hz, 1H), 4.88 (ddd, J=11.4, 8.8, 2.8 Hz, 1H), 4.64 (d, J=11.3 Hz, 1H), 4.53 (d, J=11.3 Hz, 1H), 4.49 (dd, J=6.3, 3.4 Hz, 1H), 4.03 (qt, J=6.3, 2.7 Hz, 1H), 2.16 (td, J=7.4, 1.9 Hz, 2H), 1.54 (p, J=7.7 Hz, 2H), 1.50-1.32 (m, 2H), 1.41 (s, 3H), 1.36 (s, 3H), 1.30-1.19 (m, 3H), 1.17 (ddd, J=14.4, 11.1, 4.0 Hz, 1H), 1.15-1.07 (m, 2H), 1.01 (d, J=6.4 Hz, 3H), 0.79 (d, J=6.6 Hz, 6H), 0.76 (d, J=6.4 Hz, 3H), 0.68 (d, J=6.6 Hz, 3H). ¹³C NMR (126 MHz, CDCl₃) δ 207.50, 173.28, 172.72, 169.00, 137.95, 135.43, 128.59, 128.49, 128.34, 128.09, 127.84, 127.81, 74.24, 74.13, 71.52, 67.25, 55.43, 55.18, 52.45, 41.20, 38.61, 36.65, 27.82, 27.03, 25.86, 24.78, 23.37, 22.58, 22.43, 21.06, 14.47. HRMS (ESI) m/z calcd for C₃₆H₃₂N₂O₆ ([M+Na]⁺) 631.3719. found 631.3723.

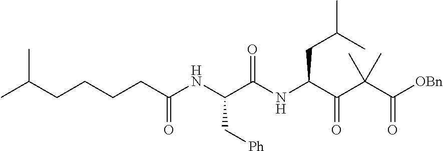 [SMILES: CC(C)CCCCC(=O)N[C@@H](Cc1ccccc1)C(=O)N[C@@H](CC(C)C)C(=O)C(C)(C)C(=O)OCc1ccccc1]

Benzyl (4S)-2,2,6-trimethyl-4-[(2S)-2-(6-methylheptanamido)-3-phenylpropanamido]-3-oxoheptanoate (17m). 97 mg (59% over two steps), colorless oil. TLC: R_{f} 0.8 (2:1 hexanes/EtOAc). IR (ZnSe) 3288.73; 3019.08; 2958.45; 2870.16; 1736.92; 1715.44; 1646.46; 1541.76; 1507.07; 1468.89; 1456.55; 1387.90; 1369.25; 1246.77; 1216.21; 1146.12; 1045.89; 909.18 ¹H NMR (500 MHz, CDCl₃) δ 7.32-7.24 (m, 4H), 7.27-7.06 (m, 4H), 6.13 (d, J=9.0 Hz, 1H), 5.89 (d, J=7.7 Hz, 1H), 5.11 (d, J=12.3 Hz, 1H), 5.06 (d, J=11.9 Hz, 1H), 4.85 (dt, J=10.8, 9.0, 3.1 Hz, 1H), 4.55 (q, J=21.9, 7.5 Hz, 1H), 2.93 (t, J=7.2 Hz, 2H), 2.06 (t, J=7.6 Hz, 2H), 1.50-1.36 (m, 3H), 1.34 (s, 3H), 1.29 (s, 3H), 1.40-1.25 (m, 1H), 1.25-1.12 (m, 4H), 1.06 (ddd, J=10.0, 6.9, 5.0 Hz, 2H), 0.84-0.75 (m, 9H), 0.73 (d, 3H). ¹³C NMR (126 MHz, CDCl₃) δ 207.25, 173.19, 172.72, 170.29, 136.34, 135.37, 129.27, 128.70, 128.66, 128.64, 128.43, 128.18, 128.10, 127.03, 67.28, 54.99, 54.07, 52.42, 41.55, 38.59, 37.67, 36.59, 27.76, 26.96, 25.81, 24.64, 23.39, 22.58, 22.25, 21.19. HRMS (ESI) m/z calcd for C₃₄H₄₈N₂O₅ ([M+Na]⁺) 587.3445. found 587.3461.

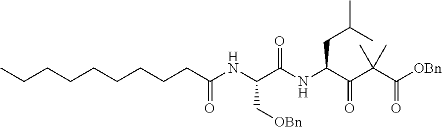 [SMILES: CCCCCCCCCC(=O)N[C@@H](COCc1ccccc1)C(=O)N[C@@H](CC(C)C)C(=O)C(C)(C)C(=O)OCc1ccccc1]

Benzyl (4S)-4-[(2S)-3-(benzyloxy)-2-decanamidopropanamido]-2,2,6-trimethyl-3-oxoheptanoate (17q). 39 mg (59% over two steps), colorless oil. TLC: R_{f} 0.65 (2:1 hexanes/EtOAc). IR (ZnSe) 3281.30; 3066.58; 3034.90; 2926.07; 2855.39; 2251.82; 1740.77; 1715.90; 1498.42; 1467.31; 1455.52; 1388.06; 1368.56; 1257.46; 1213.22; 1142.09; 1120.01; 1047.51; 907.89; 730.73 ¹H NMR (500 MHz, CDCl₃) δ 7.31-7.17 (m, 10H), 6.79 (d, J=9.1 Hz, 1H), 6.19 (d, J=6.5 Hz, 1H), 5.07 (p, J=13.5, 12.9 Hz, 2H), 4.95-4.87 (m, 1H), 4.47 (s, 1H), 4.43 (ddd, J=10.6, 5.4, 2.9 Hz, 1H), 3.75 (dd, J=9.3, 4.0 Hz, 1H), 3.31 (dd, J=9.2, 7.7 Hz, 1H), 2.12 (dd, J=8.9, 6.8 Hz, 2H), 1.54 (q, J=7.2 Hz, 2H), 1.51-1.40 (m, 1H), 1.38 (s, 2H), 1.35 (d, J=2.6 Hz, 3H), 1.34-1.14 (m, 15H), 0.85-0.76 (m, 6H), 0.75-0.69 (m, 3H). ¹³C NMR (126 MHz, CDCl₃) δ 207.43, 173.27, 172.77, 169.66, 137.29, 135.39, 128.62, 128.51, 128.38, 128.11, 127.99, 127.83, 73.61, 69.33, 67.28, 55.08, 52.65, 52.04, 52.01, 41.41, 36.57, 31.86, 29.42, 29.34, 29.27, 25.56, 24.71, 23.40, 22.67, 22.26, 21.18, 14.11. HRMS (ESI) m/z calcd for C₃₇H₅₄N₂O₆ ([M+Na]⁺) 645.3893. found 645.3880.

Boc Deprotection of Dipeptides and N-Acylation with Anhydrides (17n, 17o, 17p, 17r).

General Protocol. In a roundbottom flask, Boc-protected dipeptide O-ketoester 16 (1 equiv) was dissolved in a mixture of anhyd CH₂Cl₂ and trifluoroacetic acid (4:1) and the reaction was stirred until complete conversion as determined by LC-MS analysis. The solvent was removed by rotary evaporation and the crude amine was carried forward directly to the next step without further purification.

In a glass vial, the crude amine was dissolved in anhyd CH₂Cl₂ (0.1 M) and cooled at 0° C. The appropriate anhydride (1.1 equiv), was added to the stirring mixture. Triethylamine (3 equiv) was added dropwise. The reaction was allowed to warm to rt and stirred until complete conversion as determined by LC-MS analysis. The mixture was diluted with CH₂Cl₂, washed with water (2×) and brine, dried (Na₂SO₄), filtered, and concentrated by rotary evaporation. Purification by silica flash chromatography (7:3 hexanes/EtOAc) afforded the N-acyl dipeptide β-ketoester 17.

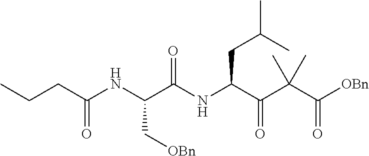 [SMILES: CCCC(=O)N[C@@H](COCc1ccccc1)C(=O)N[C@@H](CC(C)C)C(=O)C(C)(C)C(=O)OCc1ccccc1]

Benzyl (S)-4-((S)-3-(benzyloxy)-2-butyramidopropanamido)-2,2,6-trimethyl-3-oxoheptanoate (17n). 31 mg (88% over two steps), colorless oil. TLC: R_{f} 0.4 (2:1 hexanes/EtOAc). IR (ZnSe) 3308.60; 2944.30; 2832.71; 1643.03; 1453.81; 1410.98; 1112.82; 1023.19 ¹H NMR (600 MHz, CDCl₃) δ 7.38-7.26 (m, 10H), 7.10 (d, J=9.0 Hz, 1H), 6.94 (d, J=7.5 Hz, 1H), 5.21-5.15 (m, 1H), 5.13 (d, J=12.4 Hz, 1H), 5.00 (tdd, J=9.1, 6.4, 4.5 Hz, 1H), 4.67 (td, J=7.4, 4.5 Hz, 1H), 4.52 (s, 2H), 3.76 (dd, J=9.4, 4.5 Hz, 1H), 3.45 (dd, J=9.4, 7.5 Hz, 1H), 2.24-2.18 (m, 2H), 2.06 (s, 1H), 1.65 (dt, J=14.9, 7.4 Hz, 2H), 1.58-1.48 (m, 1H), 1.47-1.39 (m, 6H), 1.39-1.33 (m, 2H), 0.93 (td, J=7.4, 3.4 Hz, 3H), 0.85 (dd, J=6.5, 5.0 Hz, 3H), 0.79 (dd, J=16.3, 6.7 Hz, 3H). ¹³C NMR (151 MHz, CDCl₃) δ 207.40, 177.23, 173.94, 172.76, 170.20, 137.24, 135.35, 128.61, 128.47, 128.46, 128.38, 128.10, 128.07, 127.93, 127.79, 73.48, 73.46, 69.51, 69.35, 67.28, 60.51, 55.09, 52.72, 52.66, 52.34, 52.15, 41.36, 41.18, 38.14, 24.63, 24.52, 23.36, 22.21, 22.14, 22.12, 21.10, 21.06, 21.04, 20.83, 19.02, 14.17, 13.62. HRMS (ESI) calcd for C₃₁H₄₃N₂O₆ ([M+H]⁺) 539.3121. found 539.3137.

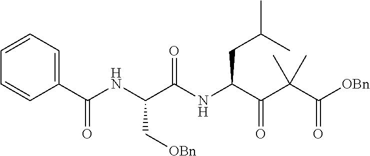 [SMILES: CC(C)C[C@H](NC(=O)[C@H](COCc1ccccc1)NC(=O)c1ccccc1)C(=O)C(C)(C)C(=O)OCc1ccccc1]

Benzyl (4S)-4-[(2S)-3-(benzyloxy)-2-(phenylformamido)propanamido]-2,2,6-trimethyl-3-oxoheptanoate (17o). 28 mg (46% over two steps), colorless oil. TLC: R_{f} 0.5 (2:1 hexanes/EtOAc). IR (ZnSe) 3290.44; 3065.09; 2957.36; 2869.01; 1741.29; 1715.53; 1539.74; 1490.43; 1468.89; 1454.90; 1387.39; 1368.40; 1313.47; 1213.44; 1141.48; 1106.37; 1045.68; 910.05 ¹H NMR (500 MHz, CDCl₃) δ 7.83-7.76 (m, 2H), 7.55-7.46 (m, 1H), 7.48-7.41 (m, 2H), 7.40-7.27 (m, 10H), 7.07 (d, J=6.2 Hz, 1H), 7.00 (d, J=9.1 Hz, 1H), 5.19 (d, J=12.4 Hz, 1H), 5.14 (d, J=12.4 Hz, 1H), 5.09-4.99 (m, 1H), 4.74-4.66 (m, 1H), 4.59 (s, 2H), 3.95 (dd, J=9.2, 4.0 Hz, 1H), 3.46 (dd, J=9.2, 8.1 Hz, 1H), 1.54 (ttd, J=11.8, 5.9, 5.1, 3.4 Hz, 1H), 1.48 (s, 3H), 1.44 (s, 3H), 1.44-1.29 (m, 2H), 0.86 (d, J=6.5 Hz, 3H), 0.79 (d, J=6.6 Hz, 3H). ¹³C NMR (126 MHz, CDCl₃) δ 207.43, 172.78, 169.68, 167.23, 137.26, 135.39, 133.68, 131.88, 128.63, 128.60, 128.55, 128.40, 128.10, 128.07, 128.04, 127.88, 127.14, 73.71, 69.37, 67.32, 55.12, 52.76, 52.41, 41.40, 24.75, 23.38, 22.30, 21.19. HRMS (ESI) m/z calcd for C₃₄H₄₀N₂O₆ ([M+Na]⁺) 595.2774. found 595.2784.

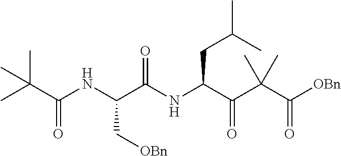 [SMILES: CC(C)C[C@H](NC(=O)[C@H](COCc1ccccc1)NC(=O)C(C)(C)C)C(=O)C(C)(C)C(=O)OCc1ccccc1]

Benzyl (4S)-4-[(2S)-3-(benzyloxy)-2-(2,2-dimethylpropanamido)propanamido]-2,2,6-trimethyl-3-oxoheptanoate (17p). 33 mg (56% over two steps), colorless oil. TLC: R_{f} 0.6 (2:1 hexanes/EtOAc). IR (ZnSe) 3427.84; 3295.15; 2960.11; 2870.41; 2251.73; 1740.32; 1715.45; 1679.56; 1642.87; 1498.94; 1468.63; 1388.60; 1367.38; 1259.84; 1206.84; 1142.93; 1106.75; 1047.16; 908.26 ¹H NMR (500 MHz, CDCl₃) δ 7.37-7.27 (m, 10H), 6.90 (d, J=9.0 Hz, 1H), 6.52 (d, J=6.1 Hz, 1H), 5.18 (d, J=12.3 Hz, 1H), 5.13 (d, J=12.4 Hz, 1H), 4.97 (ddd, J=10.7, 9.0, 3.1 Hz, 1H), 4.60-4.51 (m, 2H), 4.46 (ddd, J=7.6, 6.1, 4.1 Hz, 1H), 3.81 (dd, J=9.2, 4.1 Hz, 1H), 3.38 (dd, J=9.2, 7.8 Hz, 1H), 1.57-1.45 (m, 1H), 1.46 (s, 3H), 1.42 (s, 3H), 1.41-1.27 (m, 2H), 1.20 (d, J=1.3 Hz, 9H), 0.85 (d, J=6.4 Hz, 3H), 0.79 (d, J=6.6 Hz, 3H). 13C NMR (126 MHz, CDCl₃) δ 207.46, 178.68, 172.77, 169.86, 137.38, 135.41, 128.61, 128.52, 128.37, 128.11, 128.00, 127.97, 127.94, 127.78, 73.53, 69.10, 67.28, 55.06, 52.69, 51.98, 51.94, 41.36, 38.79, 27.47, 27.42, 24.70, 23.40, 22.34, 22.29, 21.20. HRMS (ESI) m/z calcd for C₃₂H₄₄N₂O₆ ([M+Na]⁺) 575.3112. found 575.3097.

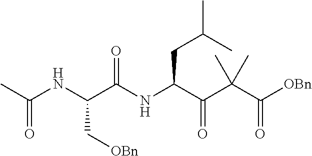 [SMILES: CC(=O)N[C@@H](COCc1ccccc1)C(=O)N[C@@H](CC(C)C)C(=O)C(C)(C)C(=O)OCc1ccccc1]

Benzyl (4S)-4-[(2S)-3-(benzyloxy)-2-acetamidopropanamido]-2,2,6-trimethyl-3-oxoheptanoate (17r). 29 mg (53% over two steps), colorless oil. TLC: R_{f} 0.4 (2:1 hexanes/EtOAc). IR (ZnSe) 3292.00; 3067.21; 2958.98; 2870.40; 2251.22; 1715.95; 1649.60; 1538.29; 1469.02; 1455.29; 1388.11; 1371.49; 1260.51; 1144.26; 909.81 ¹H NMR (500 MHz, CDCl₃) δ 7.25 (m, 10H), 6.80 (d, J=9.1 Hz, 1H), 6.24 (d, J=6.6 Hz, 1H), 5.14-5.00 (m, 2H), 4.92 (ddd, J=10.6, 9.0, 3.3 Hz, 1H), 4.47 (s, 2H), 4.43 (td, J=7.1, 4.0 Hz, 1H), 3.74 (dd, J=9.2, 4.0 Hz, 1H), 3.30 (dd, J=9.1, 7.9 Hz, 1H), 1.93 (s, 3H), 1.49-1.41 (m, 1H), 1.38 (s, 3H), 1.35 (s, 3H), 1.35-1.19 (m, 2H), 0.79 (d, J=6.4 Hz, 3H), 0.73 (d, J=6.6 Hz, 3H). ¹³C NMR (126 MHz, CDCl₃) δ 207.47, 172.76, 170.09, 169.57, 137.22, 135.38, 128.63, 128.52, 128.40, 128.10, 128.05, 128.03, 73.64, 67.29, 55.10, 52.68, 52.19, 52.16, 41.41, 24.72, 23.39, 23.20, 22.26, 21.17. HRMS (ESI) m/z calcd for C₂₉H₃₈N₂O₆ ([M+Na]⁺) 533.2620. found 533.2628.

### Global Deprotection of β-Ketoesters by Pd-Catalyzed Hydrogenolysis (11f-11r).

General Protocol. In a roundbottom flask, protected β-ketoester 17 (1 equiv) was dissolved in MeOH (0.1 M). Pd/C (1:1 w/w) was added and the mixture was stirred under H2 at atmospheric pressure at rt for 2 h until complete conversion as determined by LC-MS analysis. The mixture was filtered through a 0.2 μm syringe filter to remove Pd/C and concentrated by rotary evaporation. The crude product was dissolved in 50:50 CH₃CN/water. Purification by HPLC (45-95% CH₃CN in H₂O with 0.1% TFA) afforded the free β-ketoacid 11.

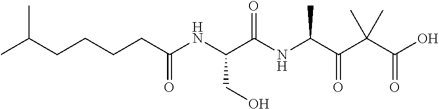 [SMILES: CC(C)CCCCC(=O)N[C@@H](CO)C(=O)N[C@@H](C)C(=O)C(C)(C)C(=O)O]

(4S)-4-[(2S)-3-Hydroxy-2-(6-methylheptanamido)propanamido]-2,2-dimethyl-3-oxopentanoic acid (11f). 5 mg (37%), white powder. IR (ZnSe) 2945.08; 2832.84; 2509.14; 2231.98; 2071.84; 1658.74; 1449.50; 1417.44; 1119.75; 1023.72 ¹H NMR (500 MHz, CDCl₃) δ 7.46 (d, J=8.7 Hz, 1H), 7.18 (d, J=7.6 Hz, 1H), 5.04-4.93 (m, 1H), 4.61-4.51 (m, 1H), 3.91 (dd, J=11.6, 4.2 Hz, 1H), 3.73 (dd, J=11.6, 5.1 Hz, 1H), 2.27 (qd, J=9.8, 9.1, 5.4 Hz, 2H), 1.61 (tt, J=15.2, 7.7 Hz, 2H), 1.55-1.47 (m, 1H), 1.45-1.24 (m, 11H), 1.21-1.14 (m, 2H), 0.87-0.82 (m, 6H). ¹³C NMR (126 MHz, CDCl₃) δ 207.68, 176.14, 170.50, 170.24, 54.59, 53.91, 52.96, 38.58, 36.37, 27.80, 27.01, 25.83, 22.57. HRMS (ESI) m/z calcd for C₁₈H₃₂N₂O₆ ([M+Na]⁺) 395.2153. found 395.2158.

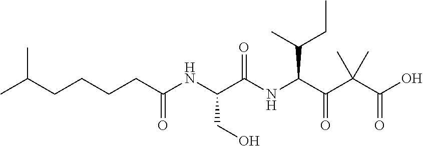 [SMILES: CCC(C)[C@H](NC(=O)[C@H](CO)NC(=O)CCCCC(C)C)C(=O)C(C)(C)C(=O)O]

(4S)-4-[(2S)-3-Hydroxy-2-(6-methylheptanamido)propanamido]-2,2,5-trimethyl-3-oxoheptanoic acid (11g). 12 mg (57%), white powder. IR (ZnSe) 3397.67; 2959.73; 2932.27; 2873.51; 2474.07; 2415.21; 2366.69; 2073.45; 1707.14; 1635.52; 1463.50; 1385.15; 1367.00; 1263.8; 1177.16; 1151.11; 1121.44; 1060.78; 978.56 ¹H NMR (500 MHz, CDCl₃) δ 7.45 (d, J=7.8 Hz, 1H), 7.13 (d, J=10.0 Hz, 1H), 4.85 (dd, J=10.1, 6.1 Hz, 1H), 4.62 (dt, J=7.8, 3.9 Hz, 1H), 4.08-3.97 (m, 1H), 3.73 (dd, J=11.9, 4.4 Hz, 1H), 2.30 (m, 2H), 2.04 (m, 1H), 1.67-1.56 (m, 2H), 1.59-1.47 (m, 1H), 1.40 (s, 3H), 1.38 (s, 3H), 1.38-1.23 (m, 3H), 1.21-1.11 (m, 3H), 0.90-0.81 (m, 12H). ¹³C NMR (126 MHz, CDCl₃) δ 206.42, 176.00, 174.77, 170.75, 54.90, 54.30, 36.29, 36.19, 22.56, 15.91. HRMS (ESI) m/z calcd for C₂₁H₃₈N₂O₆ ([M+Na]⁺) 437.2613. found 437.2628.

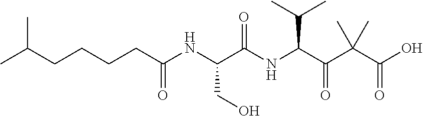 [SMILES: CC(C)CCCCC(=O)N[C@@H](CO)C(=O)N[C@H](C(=O)C(C)(C)C(=O)O)C(C)C]

(4S)-4-[(2S)-3-Hydroxy-2-(6-methylheptanamido)propanamido]-2,2,5-trimethyl-3-oxohexanoic acid (11h). 5 mg (30%) as a white powder. IR (ZnSe) 3371.26; 2243.96; 2216.11; 2071.33; 1121.68; 1092.70; 973.54 ¹H NMR (500 MHz, CD₃OD) δ 4.56-4.46 (m, 1H), 3.85-3.74 (m, 2H), 2.36-2.29 (m, 2H), 2.29-2.20 (m, 1H), 1.70-1.53 (m, 3H), 1.45 (s, 3H), 1.40 (s, 3H), 1.43-1.35 (m, 2H), 1.29-1.21 (m, 2H), 0.96-0.92 (m, 9H), 0.90 (d, J=6.8 Hz, 3H). ¹³C NMR (126 MHz, CD₃OD) δ 206.90, 175.10, 170.97, 61.31, 58.45, 55.13, 38.50, 35.53, 29.77, 27.65, 26.74, 25.80, 21.64, 21.32, 18.98, 15.92. HRMS (ESI) m/z calcd for C₂₀H₃₆N₂O₆ ([M+Na]⁺) 423.2472. found 423.2471.

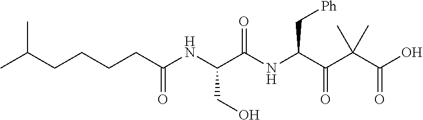 [SMILES: CC(C)CCCCC(=O)N[C@@H](CO)C(=O)N[C@@H](Cc1ccccc1)C(=O)C(C)(C)C(=O)O]

(4S)-4-[(2S)-3-Hydroxy-2-(6-methylheptanamido)propanamido]-2,2-dimethyl-3-oxo-5-phenylpentanoic acid (11i). 5 mg (35%), white powder. IR (ZnSe) 3341.97; 2944.54; 2832.42; 2523.88; 2045.04; 1651.46; 1449.51; 1417.41; 1114.80; 1023.54 ¹H NMR (500 MHz, CD₃OD) δ 7.32-7.15 (m, 5H), 5.23-5.16 (m, 1H), 4.45-4.34 (m, 1H), 3.76-3.63 (m, 1H), 3.66-3.57 (m, 1H), 3.15 (dd, J=13.9, 4.9 Hz, 1H), 2.84-2.75 (m, 1H), 2.30-2.20 (m, 2H), 1.65-1.50 (m, 3H), 1.36 (s, 3H), 1.42-1.29 (m, 1H), 1.30 (s, 3H), 1.28-1.17 (m, 3H), 0.90 (d, J=6.7 Hz, 6H). ¹³C NMR (126 MHz, CD₃OD) δ 206.66, 174.99, 170.13, 129.12, 127.97, 61.50, 55.03, 54.77, 48.11, 47.94, 47.77, 47.60, 47.43, 47.26, 47.09, 35.53, 27.62, 26.73, 25.66, 21.60. HRMS (ESI) m/z calcd for C24H36N2O6 ([M+Na]⁺) 471.2486. found 471.2471.

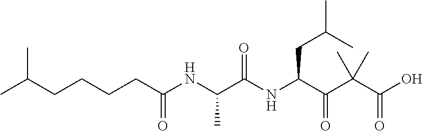 [SMILES: CC(C)CCCCC(=O)N[C@@H](C)C(=O)N[C@@H](CC(C)C)C(=O)C(C)(C)C(=O)O]

(4S)-2,2,6-Trimethyl-4-[(2S)-2-(6-methylheptanamido)propanamido]-3-oxoheptanoic acid (11j). 5.3 mg (32%), white powder. IR (ZnSe) 3370.84; 2954.21; 2835.25; 2360.07; 2332.17; 1676.56; 1652.55; 1456.36; 1418.69; 1205.35; 1182.26; 1144.59; 1116.63; 1032.43 ¹H NMR (500 MHz, CD₃OD) δ 5.05-4.98 (m, 1H), 4.36 (dq, J=21.4, 7.0 Hz, 1H), 2.28-2.16 (m, 2H), 1.62-1.47 (m, 5H), 1.44-1.27 (m, 9H), 1.22 (dt, J=10.8, 6.9 Hz, 2H), 0.99-0.84 (m, 12H). ¹³C NMR (126 MHz, CD₃OD) δ 207.71, 174.92, 174.66, 173.11, 55.29, 54.43, 52.38, 48.76, 40.56, 38.47, 36.89, 35.37, 27.62, 26.70, 25.77, 24.37, 22.42, 21.59, 21.45, 21.22, 20.24, 17.08, 16.60, 16.37. HRMS (ESI) m/z calcd for C₂₁H₃₈N₂O₅ ([M+Na]⁺) 421.2674. found 421.2678.

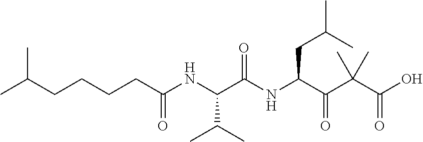 [SMILES: CC(C)CCCCC(=O)N[C@H](C(=O)N[C@@H](CC(C)C)C(=O)C(C)(C)C(=O)O)C(C)C]

(4S)-2,2,6-Trimethyl-4-[(2S)-3-methyl-2-(6-methylheptanamido)butanamido]-3-oxoheptanoic acid (11k). 5 mg (49%), white powder. IR (ZnSe) 3300.06; 2958.49; 2934.10; 2872.23; 2360.41; 2341.89; 1638.74; 1558.29; 1541.30; 1466.47; 1033.43 ¹H NMR (500 MHz, CDCl₃) δ 6.84 (s, 1H), 6.72 (s, 1H), 5.06 (td, J=9.3, 3.8 Hz, 1H), 4.36-4.26 (m, 1H), 2.26 (td, J=7.9, 7.5, 1.6 Hz, 2H), 2.04 (dt, J=13.6, 6.7 Hz, 1H), 1.67-1.57 (m, 2H), 1.57-1.50 (m, 3H), 1.49 (s, 3H), 1.44 (s, 3H), 1.37-1.27 (m, 2H), 1.23-1.17 (m, 2H), 0.99-0.90 (m, 12H), 0.89-0.85 (m, 6H). ¹³C NMR (126 MHz, CDCl₃) δ 208.00, 174.39, 171.74, 54.97, 38.57, 36.59, 31.34, 30.80, 27.82, 26.98, 26.01, 24.86, 23.41, 22.55, 21.25, 19.08, 18.24. HRMS (ESI) m/z calcd for C₂₃H₄₂N₂O₅ ([M+Na]⁺) 449.2986. found 449.2991.

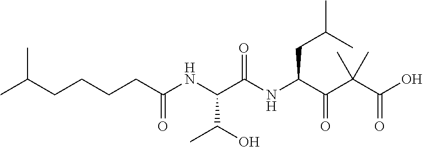 [SMILES: CC(C)CCCCC(=O)N[C@H](C(=O)N[C@@H](CC(C)C)C(=O)C(C)(C)C(=O)O)C(C)O]

(4S)-4-[(2S)-3-Hydroxy-2-(6-methylheptanamido)butanamido]-2,2,6-trimethyl-3-oxoheptanoic acid (111). 7 mg (61%), white powder. IR (ZnSe) 3301.81; 2957.21; 2935.18; 2871.57; 2360.23; 2341.71; 1701.57; 1637.86; 1541.15; 1469.34; 1385.54; 1368.45; 1209.32; 1183.63; 1152.04; 1033.73 ¹H NMR (500 MHz, CD₃OD) S 5.06 (dt, J=10.6, 5.3 Hz, 1H), 4.31 (t, J=6.3 Hz, 1H), 4.14-3.99 (m, 1H), 2.30 (m, 2H), 1.67-1.46 (m, 5H), 1.39 (m, Hz, 9H), 1.28-1.19 (m, 2H), 1.17 (d, J=6.3 Hz, 3H), 0.92 (tt, J=9.6, 5.4 Hz, 12H). ¹³C NMR (126 MHz, CD₃OD) δ 207.70, 175.02, 170.73, 124.90, 66.93, 58.57, 54.56, 52.25, 40.65, 38.49, 35.56, 27.64, 26.71, 25.82, 24.40, 22.52, 21.59, 21.48, 21.26, 20.20, 18.85. HRMS (ESI) m/z calcd for C₂₂H₄₀N₂O₆ ([M+Na]⁺) 451.2788. found 451.2784.

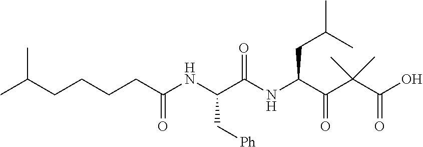 [SMILES: CC(C)CCCCC(=O)N[C@@H](Cc1ccccc1)C(=O)N[C@@H](CC(C)C)C(=O)C(C)(C)C(=O)O]

(4S)-2,2,6-Trimethyl-4-[(2S)-2-(6-methylheptanamido)-3-phenylpropanamido]-3-oxoheptanoic acid (11m). 3 mg (22%), white powder. IR (ZnSe) 2957.08; 2931.09; 2869.27; 2480.47; 2248.03; 2073.03; 1707.06; 1633.92; 1466.36; 1264.44; 1123.70; 1051.18; 979.30. ¹H NMR (500 MHz, CD₃OD) δ 8.22 (d, J=9.0 Hz, 1H), 8.08 (d, J=8.4 Hz, OH), 7.35-7.19 (m, 5H), 5.05 (dd, J=8.9, 5.1 Hz, 1H), 4.69 (dd, J=10.4, 5.3 Hz, 1H), 3.14 (dd, J=14.1, 5.5 Hz, 1H), 2.87 (dd, J=13.9, 9.5 Hz, 1H), 2.18 (t, J=7.6 Hz, 2H), 1.74-1.65 (m, 1H), 1.58-1.46 (m, 4H), 1.40 (s, 3H), 1.36 (s, 3H), 1.27-1.13 (m, 5H), 1.00-0.84 (m, 12H). ¹³C NMR (126 MHz, CD₃OD) δ 207.59, 174.83 (d, J=23.6 Hz), 171.70, 137.10, 128.92, 128.02, 126.32, 54.23, 52.30, 40.65, 38.48, 37.23, 35.48, 27.57, 26.57, 25.82, 24.44, 22.56, 21.57 (d, J=14.5 Hz), 21.22, 20.25. HRMS (ESI) m/z calcd for C₂₇H₄₂N₂O₅ ([M+Na]⁺) 497.2979. found 497.2991.

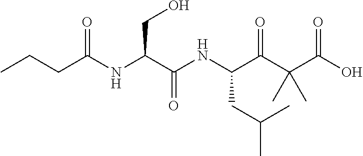 [SMILES: CCCC(=O)N[C@@H](CO)C(=O)N[C@@H](CC(C)C)C(=O)C(C)(C)C(=O)O]

(4S)-4-((S)-2-Butyramido-3-hydroxypropanamido)-2,2,6-trimethyl-3-oxoheptanoic acid (11n). 50 mg (50%), white powder. IR (ZnSe) 3304.07; 3083.36; 2959.36; 2872.77; 1708.40; 1542.55; 1468.67; 1387.64; 1369.53; 1258.17; 1208.09; 1166.41; 1044.70 ¹H NMR (600 MHz, CDCl₃) δ 7.49 (dd, J=22.8, 8.8 Hz, 1H), 7.35 (d, J=9.4 Hz, 1H), 4.94 (td, J=9.5, 4.7 Hz, 1H), 4.60 (dt, J=8.6, 4.5 Hz, 1H), 3.84 (dd, J=11.8, 4.4 Hz, 1H), 3.72-3.63 (m, 2H), 2.22 (t, J=7.5 Hz, 2H), 1.65-1.40 (m, 4H), 1.36 (s, 2H), 1.31 (d, J=10.1 Hz, 3H), 1.17 (t, J=7.0 Hz, 2H), 0.92-0.81 (m, 10H). ¹³C NMR (151 MHz, CDCl₃) δ 213.87, 207.66, 207.21, 175.98, 175.69, 174.87, 174.14, 170.95, 170.88, 170.37, 62.98, 62.96, 62.07, 58.46, 56.05, 54.93, 54.47, 54.44, 54.06, 53.94, 53.34, 53.15, 41.36, 41.24, 39.51, 38.25, 38.09, 37.96, 37.66, 25.16, 24.66, 24.63, 23.37, 23.32, 22.38, 22.11, 21.49, 21.47, 21.36, 21.34, 21.31, 19.14, 19.06, 19.03, 18.29, 17.75, 13.65, 13.62. HRMS (ESI) m/z calcd for C₁₇H₃₀N₂O₆ ([M+Na]⁺) 381.2002. found 381.2000.

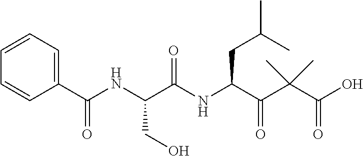 [SMILES: CC(C)C[C@H](NC(=O)[C@H](CO)NC(=O)c1ccccc1)C(=O)C(C)(C)C(=O)O]

(4S)-4-[(2S)-3-Hydroxy-2-(phenylformamido)propanamido]-2,2,6-trimethyl-3-oxoheptanoic acid (110). 3 mg (31%), white powder. IR (ZnSe) 3393.73; 2956.96; 2833.11; 2360.54; 2340.99; 1683.58; 1663.07; 1647.30; 1635.88; 1558.69; 1540.57; 1522.03; 1033.14. ¹H NMR (500 MHz, CD₃OD) δ 7.89 (d, J=7.4 Hz, 2H), 7.57 (q, J=6.9 Hz, 1H), 7.49 (td, J=7.7, 5.0 Hz, 3H), 5.10 (dt, J=12.9, 4.5 Hz, 1H), 4.73-4.64 (m, 1H), 3.60 (dd, J=11.2, 4.9 Hz, 2H), 3.53 (dd, J=11.2, 5.9 Hz, 2H), 1.76-1.66 (m, 1H), 1.55 (ddp, J=14.4, 9.8, 4.9 Hz, 2H), 1.44 (d, J=6.4 Hz, 3H), 1.36 (d, J=6.3 Hz, 3H), 0.94 (d, J=6.6 Hz, 6H). ¹³C NMR (126 MHz, CD₃OD) δ 131.52, 128.17, 127.10, 72.45, 63.00, 55.85, 54.55, 52.94, 40.58, 24.45, 22.09, 21.47, 21.28, 20.25. HRMS (ESI) m/z calcd for C₂₀H₂₈N₂O₆ ([M+Na]⁺) 415.1844. found 415.1845.

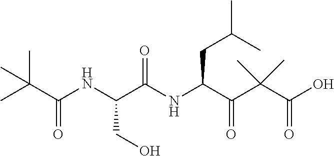 [SMILES: CC(C)C[C@H](NC(=O)[C@H](CO)NC(=O)C(C)(C)C)C(=O)C(C)(C)C(=O)O]

(4S)-4-[(2S)-2-(2,2-Dimethylpropanamido)-3-hydroxypropanamido]-2,2,6-trimethyl-3-oxoheptanoic acid (11p). 4.8 mg (44%), white powder. IR (ZnSe) 3363.23; 2964.79; 2871.07; 2359.29; 2328.27; 1733.62; 1646.02; 1558.07; 1540.65; 1521.84; 1507.50; 1465.22; 1396.23; 1261.49; 1158.89; 1033.23. ¹H NMR (500 MHz, CD₃OD) δ 7.93 (d, J=8.9 Hz, 1H), 7.18 (d, J=7.4 Hz, 1H), 4.99-4.91 (m, 1H), 4.39-4.27 (m, 1H), 3.72-3.62 (m, 2H), 1.55 (dq, J=13.2, 6.5 Hz, 1H), 1.45-1.36 (m, 2H), 1.33-1.27 (s, 3H), 1.26-1.21 (s, 3H), 1.12 (s, 9H), 0.87-0.79 (m, 6H). ¹³C NMR (126 MHz, CD₃OD) δ 207.75, 179.98, 175.06, 61.43, 55.28, 54.59, 52.35, 40.67, 38.40, 26.27, 24.42, 22.49, 22.26, 21.24, 20.24. HRMS (ESI) m/z calcd for C₁₈H₃₂N₂O₆ ([M+Na]⁺) 395.2172. found 395.2158.

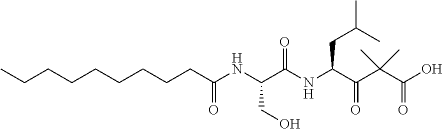 [SMILES: CCCCCCCCCC(=O)N[C@@H](CO)C(=O)N[C@@H](CC(C)C)C(=O)C(C)(C)C(=O)O]

(4S)-4-[(2S)-2-Decanamido-3-hydroxypropanamido]-2,2,6-trimethyl-3-oxoheptanoic acid (11q). 12 mg (84%), white powder. IR (ZnSe) 3383.07; 2956.78; 2926.12; 2855.27; 2485.03; 2435.71; 2362.98; 2073.78; 1708.24; 1635.75; 1467.13; 1387.37; 1368.85; 1272.39; 1163.51; 1121.38; 1054.15; 978.75. ¹H NMR (500 MHz, CD₃OD) δ 5.09 (dd, J=9.5, 4.7 Hz, 1H), 4.48 (dt, J=21.1, 5.7 Hz, 1H), 3.78 (dtd, J=21.5, 11.4, 11.0, 6.7 Hz, 2H), 2.31 (td, J=7.4, 3.9 Hz, 2H), 1.78-1.61 (m, 3H), 1.55 (tq, J=7.5, 4.2 Hz, 2H), 1.46 (s, 3H), 1.41-1.27 (m, 15H), 0.97 (dq, J=13.3, 7.2, 6.7 Hz, 9H). ¹³C NMR (126 MHz, CDCl₃) δ 213.61, 173.98, 171.06, 62.92, 55.92, 53.71, 39.63, 37.65, 36.51, 31.86, 29.41 (t, J=5.0 Hz), 29.37-29.17 (m), 25.63, 25.18, 23.35, 22.67, 21.36, 19.16, 17.76, 14.11. HRMS (ESI) m/z calcd for C₂₃H₄₂N₂O₆ ([M+Na]⁺) 465.2947. found 465.2941.

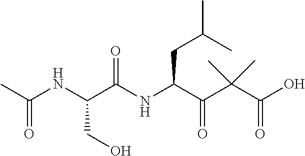 [SMILES: CC(=O)N[C@@H](CO)C(=O)N[C@@H](CC(C)C)C(=O)C(C)(C)C(=O)O]

(4S)-4-[(2S)-2-Acetamido-3-hydroxypropanamido]-2,2,6-trimethyl-3-oxoheptanoic acid (11r). 5.3 mg (49%), white powder. IR (ZnSe) 3450.46; 2958.94; 2922.90; 2480.26; 2225.89; 2073.60; 1710.18; 1643.59; 1468.55; 1433.82; 1369.61; 1270.42; 1163.76; 1121.81; 1060.21; 979.48. ¹H NMR (500 MHz, CD₃OD) δ 5.08 (dd, J=9.8, 4.5 Hz, 1H), 4.45 (t, J=5.7 Hz, 1H), 3.83-3.72 (m, 2H), 2.06 (s, 3H), 1.78-1.66 (m, 1H), 1.63-1.51 (m, 2H), 1.46 (s, 3H), 1.39 (s, 3H), 0.98 (d, J=6.7 Hz, 6H). ¹³C NMR (126 MHz, CD₃OD) δ 207.69, 175.01, 172.11, 170.50, 61.56, 55.49, 54.54, 52.46, 40.59, 24.42, 22.51, 21.48, 21.29, 21.11, 20.28. HRMS (ESI) m/z calcd for C₁₅H₂₆N₂O₆ ([M+Na]⁺) 353.1704. found 353.1689.

### Stability of β-Ketoacid Substrates

It was reported that β-ketoacid 11n can undergo spontaneous decarboxylation during isolation and characterization according to below scheme [Zabala, D.; Cartwright, J. W.; Roberts, D. M.; Law, B. J. C.; Song, L.; Samborskyy, M.; Leadlay, P. F.; Micklefield, J.; Challis, G. L. (2016) A flavin-dependent decarboxylase-dehydrogenase-monooxygenase assembles the warhead of α,β-epoxyketone proteasome inhibitors. J. Am. Chem. Soc. 138, 4342-4345.].

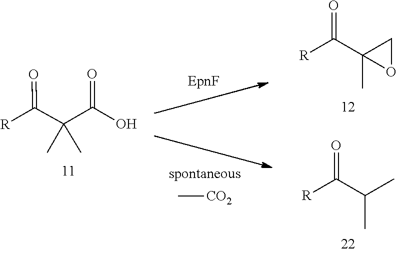

To assess rates of decarboxylation under conditions relevant to storage, biochemical assays, and cellular assays, we investigated the stability of eponemycin-based β-ketoacids 11a and 11c under a variety of conditions as shown in below Table C.

TABLE C

| substrate | form | T | | t_{1/2} (% remaining at last time point) | |
|---|---|---|---|---|---|
| 11a | neat | 25° | C. | 120 | h |
| | neat | 4° | C. | >14 | d (80%) |
| | neat | −20° | C. | >14 | d (95%) |
| | PBS | 37° | C. | 4.5 | h |
| | RPMI | 37° | C. | 2.5 | h |
| 11c | PBS | 37° | C. | 0.25 | h |

For samples stored neat, β-ketoacid 11 (lyophilized powder) was aliquoted into 1.5-2 mg samples in glass vials and placed under Ar. Samples were stored on the bench top (20-25° C.), in at 4° C. refrigerator or in a −20° C. freezer. At each time point, an entire aliquot was dissolved in CD₃OD (600 μL) analyzed by ¹H-NMR for decarboxylation to the corresponding isopropyl ketone byproduct 22. The degree of decarboxylation was determined by comparing integrations of two singlet peaks at 1.40 and 1.33 ppm, arising from the α,α-dimethyl groups in the β-ketoacid 11, to two doublet peaks at 1.09 and 1.06 ppm, arising from the isopropyl methyl groups in the decarboxylation product 22. A septuplet corresponding to the methine proton of the isopropyl group was also observed at 2.91 ppm.

For samples stored in PBS, aliquots of β-ketoacid 11 were dissolved in deuterated PBS (600 μL) and incubated in a 37° C. shaking incubator. At each time point, one aliquot was removed and analyzed by ¹H-NMR as above. NMR changes observed in deuterated PBS were similar to those observed in CD₃OD above.

For samples stored in RPMI, β-ketoacid 11 (lyophilized powder) was dissolved in EtOH to a working stock concentration of 20 mM. The sample was diluted in RPMI to 100 μM (1000 μL) and incubated at 37° C. At each time point, an aliquot (50 μL) was removed from the master sample, transferred to a 96-well plate, quenched with 4 vol MeOH, centrifuged at 4,000 g to precipitate debris, and immediately analyzed by SPE-MS/MS to quantitate β-ketoacid 11 remaining.

Results: It was found that the parent substrate 11a was fairly stable when stored neat at low temperatures, although rates of decarboxylation increased in buffer or media at 37° C. The rate of decarboxylation of the β′-hydroxy congener 11c proved to be considerably faster, although it was still stable enough to evaluate in the short timeframe of the biochemical assays.

### Biochemical Evaluation of β-Ketoacids

### Semiquantitative Epoxide Capture Assay.

Recombinant EpnF was diluted to 1 μM in 180 μL EpnF Buffer in an 1.5 mL Eppendorf tube. The β-ketoacid 11 was added at various concentrations to a total reaction volume of 200 μL. The mixture was incubated in a heated shaker at 37° C. and 225 rpm for 1 h. The reaction mixture (30 μL×3 technical replicates) was transferred to a white, opaque 96-well plate (PerkinElmer, OptiPlate-96). Each plate also contained 30 μL×3 technical replicates of EpnF Buffer as a negative control. To each well, 15 μL of 125 mM nicotinamide in EpnF Buffer was added and mixed by pipetting. The plate was sealed with an adhesive foil cover (VWR Adhesive Foil for Microplates) and heated in an oven at 80° C. for 1 h. The plate was cooled to rt and 25 μL of 15% (v/v) acetophenone in EtOH and 25 μL of 1 M potassium hydroxide in EtOH were added to each well, then mixed by pipetting. The plate was cooled on ice for 10 min, then 125 μL formic acid was added to each well. The plate was resealed and heated in an oven at 100° C. for 5 min. The plate was cooled on ice for 5-10 min, then analyzed (λₑₓ 370 nm, λₑₘ 430 nm) on a fluorescence plate reader (Molecular Devices Spectramax M3).

### Enzyme Kinetic Analysis by SPE-MS/MS Assay.

SPE-MS/MS analyses were carried out on an Agilent RapidFire 365 High-Throughput Mass Spectrometry System with 6495 triple quadrupole mass spectrometer.

Determination of initial velocity: EpnF stock at 100 μM was diluted 10-fold in EpnF Buffer to a 10 μM working solution, which was further diluted to 8 concentrations (10-0.005 μM) in EpnF Buffer (intermediate volume 1350 μL, concentrations calculated for 1500 μL theoretical final volume corresponding to addition of substrate solutions below). The 8 EpnF enzyme dilutions were aliquoted (90 μL) into the corresponding 8 tubes of PCR tube strips (12 strips, one per time point; Thermo Scientific, AB-2000). The β-ketoacid 11 (20 mM in EtOH) was diluted to 10 μM in EpnF Buffer, then 10 μL of this solution was added to each of the tubes (1 μM final concentration, 100 μL final volume) to initiate the reaction. Samples were incubated in a heated shaker at 37° C. and 225 rpm for up to 120 min, and one tube strip was removed at each time point. Each tube was quenched with 5% formic acid in EpnF Buffer (100 μL), pipetted vigorously, and flash frozen on dry ice. Samples were thawed then centrifuged to precipitate protein and transferred to a conical-bottom 96-well plate (Thermo Scientific/Nunc, 249944) for SPE-MS/MS analysis.

Measurement of Kₘ and Vₘₐₓ: Using the data from the initial velocity experiments above, an appropriate EpnF enzyme concentration was selected for each β-ketoacid 11 at which the rate of conversion to the epoxyketone 12 remained linear at 10 min (0.50 μM for 11n and 0.05 μM for all other substrates). Each substrate (20 mM in EtOH) was diluted to 1 mM in EpnF Buffer, then diluted to 8 concentrations (100-0.5 μM) in EpnF Buffer (intermediate volume 1350 μL, concentrations calculated for 1500 μL theoretical volume corresponding to addition of enzyme solutions below). The 8 substrate dilutions were aliquoted (90 μL) into the corresponding 8 tubes of PCR tube strips (12 strips, one per time point; Thermo Scientific, AB-2000). The EpnF enzyme stock solution was diluted in EpnF Buffer to the appropriate concentration for each substrate, then 10 μL of this solution was added to each of the tubes (100 μL final volume) to initiate the reaction. For the 0-min time point, 5% formic acid in EpnF Buffer was added to the β-ketoacid substrate before addition of the enzyme. Samples were incubated in a heated shaker at 37° C. and 225 rpm for up to 10 min, and one tube strip was removed at each time point. Each tube was quenched by addition of 5% formic acid in EpnF Buffer (100 μL), pipetted vigorously, and flash frozen on dry ice. Samples were thawed then centrifuged to precipitate proteins and the supernatants were transferred to a conical-bottom 96-well plate (Thermo Scientific/Nunc, 249944) for SPE-MS/MS analysis. Kₘ and Vₘₐₓ values for each substrate were calculated by Michaelis-Menten analysis.

### Discussion and Results

We adapted a reported epoxide capture assay [Nelis, H. J. C. F.; Sinsheimer, J. E. (1981) A sensitive fluorimetric procedure for the determination of aliphatic epoxides under physiological conditions. Anal. Biochem. 115, 151-157.] to 96-well plate format to detect formation of the epoxyketone products semiquantitatively. Each β-ketoacid substrate 11 was incubated with recombinant, purified EpnF (1 h), then reacted with nicotinamide and acetophenone (1 h) to form a fluorescent 1,6-naphthyridinone 24. The fluorescent signals were normalized to that resulting from the known substrate β-ketoacid 11n as a positive control [Zabala, D.; Cartwright, J. W.; Roberts, D. M.; Law, B. J. C.; Song, L.; Samborskyy, M.; Leadlay, P. F.; Micklefield, J.; Challis, G. L. (2016) A flavin-dependent decarboxylase-dehydrogenase-monooxygenase assembles the warhead of α,β-epoxyketone proteasome inhibitors. J. Am. Chem. Soc. 138, 4342-4345.].

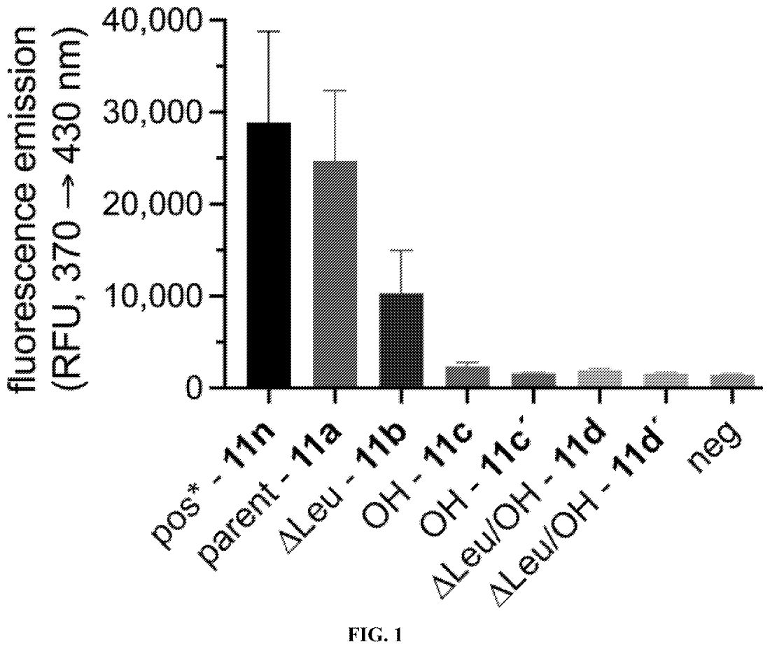

Experiments revealed that the ‘parent’ β-ketoacid 11a was converted to the corresponding epoxyketone 12a at levels comparable to that of the positive control (11n→12n). See FIG. 1. The dehydroleucine substrate 11b was also converted to epoxyketone 12b at lower levels. In contrast, no epoxide formation was detected for either diastereomer of the β′-hydroxy substrates 11c/c′ and 11d/d′. These results support a model in which the β′-hydroxy group, and possibly the leucine side-chain olefin, are introduced after epoxyketone formation in the biosynthesis of eponemycin, consistent with the sequence of tailoring reactions proposed for TMC-86A [Zabala, D.; Cartwright, J. W.; Roberts, D. M.; Law, B. J. C.; Song, L.; Samborskyy, M.; Leadlay, P. F.; Micklefield, J.; Challis, G. L. (2016) A flavin-dependent decarboxylase-dehydrogenase-monooxygenase assembles the warhead of α,β-epoxyketone proteasome inhibitors. J. Am. Chem. Soc. 138, 4342-4345.].

To obtain a quantitative assessment of EpnF substrate specificity, we developed a kinetic assay (10 min) using SPE-MS/MS (solid-phase extraction/triple quadrupole mass spectrometry; Agilent RapidFire 365 with 6495 MS) to enable direct detection of both substrates and products. In contrast to the semiquantitative epoxide capture assay above, these experiments revealed that the “parent” β-ketoacid 11a and its dehydroleucine congener 11b exhibited comparable k_{cat}/Kₘ values, as illustrated by the data in Table 1 below.

TABLE 1

| | Kₘ | k_{cat} | k_{cat}/Kₘ |
|---|---|---|---|
| Compound | (μM) | (s⁻¹) | (mM⁻¹s⁻¹) |
| 11a | 11.6 | 0.071 | 6.12 |
| 11b | 17.3 | 0.114 | 6.58 |
| 11n | 23.1 | 0.0067 | 0.29 |

Experiments were performed with 0.05 μM EpnF for 11a, b, and 0.50 μM EpnF for 11n.
Data represent mean of n = 2 technical replicates.

This difference may be due to the semiquantitative nature of the epoxide capture assay, its longer duration, and its dependence upon the rates of both epoxide formation and conversion to the fluorophore. Thus, based on the kinetic assay results, the biosynthetic order of epoxide formation and leucine γ,δ-dehydrogenation remains to be determined definitively, although previous mutagenesis experiments support a model in which epoxyketone formation precedes side-chain dehydrogenation [Zettler, J.; Zubeil, F.; Kulik, A.; Grond, S.; Kaysser, L. (2016) Epoxomicin and eponemycin biosynthesis involves gem-dimethylation and an Acyl-CoA dehydrogenase-like enzyme. ChemBioChem 17, 792-798.]. Notably, both 11a and 11b, which bear the N-isooctanoyl cap found in eponemycin, were converted ≈20-fold more efficiently than 11n, which carries the shorter N-butyryl cap found in TMC-86A.

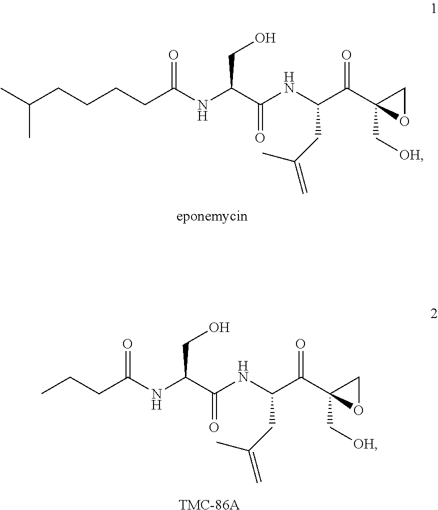

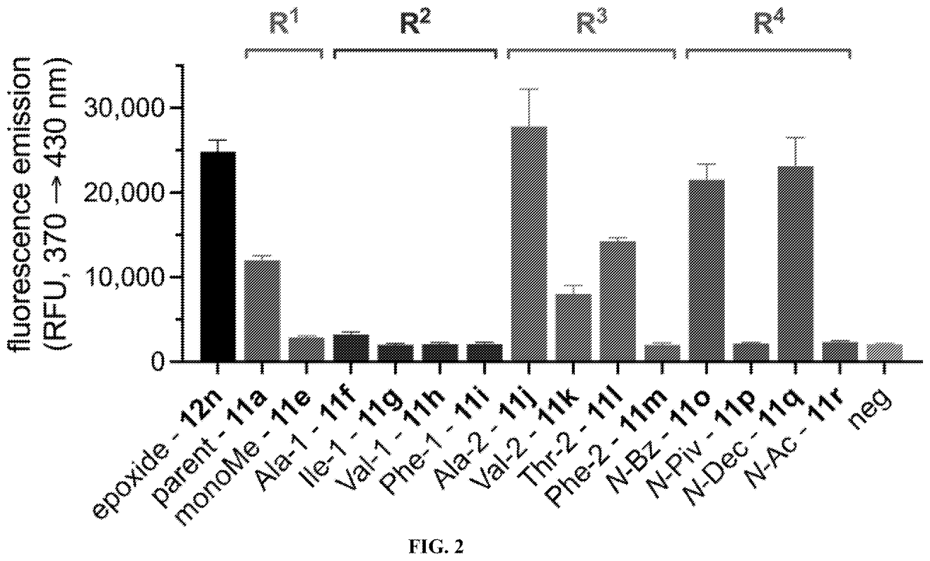

Further, we evaluated their EpnF-catalyzed conversion to the corresponding epoxyketone products 12e-r using the semiquantitative epoxide capture assay, where FIG. 2 provides a summary of the results. At the a-position (R¹), removal of the second (non-participating) α-methyl substituent was not tolerated (11e). At the first amino acid residue (R²), the leucine side chain could not be truncated to alanine (11f), replaced with a more hindered isoleucine (11g) or valine (11h) side chain, or extended to a phenylalanine side chain (11i). Certain modifications were tolerated at the second amino acid residue (R³), including truncation of the parent serine side chain to alanine (11j) and introduction of a β-substituent in a threonine side chain (11l). A valine side chain (11k), isosteric to the latter, was tolerated to a lesser degree, while an analogue with an extended phenylalanine side chain (11m) was not accepted. Some modifications of the eponemycin N-isooctanoyl group (R⁴) were also tolerated. Truncation to a butyryl chain (11n) or extension to a decanoyl chain (11q) were permitted, but not complete truncation to an acetyl group (11r). Replacement with a benzoyl group (11o) was also tolerated, but not a more sterically demanding pivaloyl group (11p).

Kinetic parameters. Kinetic parameters for EpnF-catalyzed conversion of the substrate analogues to the corresponding epoxyketones was determined, using the quantitative SPE-MS/MS assay, the results of which are provided in Table 2 below. Compared to the parent substrate 11a, the analogue with alanine at the second amino acid position (11j) was converted with similar efficiency. The analogues with threonine at the second amino acid position (11l) or an N-benzoyl cap (11o) were processed with slightly lower efficiency. Kinetic parameters could not be determined for the valine analogue 11k due to low conversion nor for the N-decanoyl analogue 11q due to solubility limitations.

TABLE 2

| | Kₘ | k_{cat} | k_{cat}/Kₘ |
|---|---|---|---|
| Compound | (μM) | (s⁻¹) | (mM⁻¹s⁻¹) |
| 11a | 11.6 | 0.071 | 6.12 |
| 11j | 4.70 | 0.025 | 5.32 |
| 11l | 13.3 | 0.016 | 1.20 |
| 11o | 6.13 | 0.0125 | 1.88 |

Experiments were performed with 0.05 μM EpnF.
Data represent mean of n = 2 technical replicates.

These experiments demonstrated that EpnF has narrow substrate specificity near the epoxyketone warhead but tolerates some modifications at the distal amino acid and N-terminal cap positions.

### Synthesis of α,β-Epoxyketones

### Enzymatic Conversion of β-Ketoacids 11 to Epoxyketones 12.

EpnF was diluted to 50 μM in EpnF Buffer (20 mM Tris HCl, pH 7.5; 100 mM NaCl; 10% glycerol) and incubated with 200 μM substrate (100 μL at 20 mM in EtOH) at a total volume of 10 mL. The mixture was incubated in a heated shaker at 37° C. and 225 rpm for 3 h, then transferred to a 50 mL Falcon tube. An equal volume of 5% formic acid in EpnF buffer was added and the mixture was centrifuged (4,000 g) for 10 min to precipitate the enzyme. The supernatant fluid was separated by semi-preparative HPLC as described in the Supporting Information. The epoxyketones 12 and decarboxylated byproducts 22 were collected separately and lyophilized prior to further analysis.

### Chemoenzymatic Synthesis of α,β-Epoxyketones (12a, 12 j, 12l, 12o)

General Protocol. β-Ketoacid 11 was diluted to 20 mM in EtOH. The reaction was carried out as described in the Methods section of the manuscript. Purification by HPLC (45-95% CH₃CN in H₂O with 0.1% TFA) afforded the epoxyketone 12.

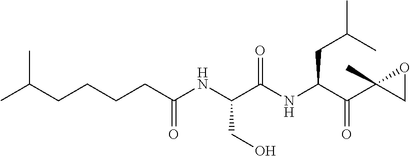 [SMILES: CC(C)CCCCC(=O)N[C@@H](CO)C(=O)N[C@@H](CC(C)C)C(=O)[C@@]1(C)CO1]

N-[(1S)-2-Hydroxy-1-{[(2S)-4-methyl-1-[(2R)-2-methyloxiran-2-yl]-1-oxopentan-2-yl]carbamoyl}ethyl]-6-methylheptanamide (12a). 1.1 mg (24%), white powder. ¹H NMR (500 MHz, CD₃OD) δ 4.63-4.56 (m, 1H), 4.45 (t, J=5.7 Hz, 1H), 3.74 (qd, J=11.3, 6.4 Hz, 2H), 3.26 (d, J=5.1 Hz, 1H), 2.94 (d, J=5.1 Hz, 1H), 2.28 (t, J=7.6 Hz, 2H), 1.74 (s, 1H), 1.68-1.45 (m, 6H), 1.41-1.26 (m, 4H), 1.22 (q, J=7.4 Hz, 2H), 0.99-0.93 (m, 6H), 0.90 (d, J=6.6 Hz, 6H). ¹³C NMR (126 MHz, CD₃OD) S 208.30, 175.04, 171.14, 78.12, 77.86, 77.60, 63.03, 61.66, 58.69, 55.05, 51.71, 50.49, 39.07, 38.49, 35.53, 27.64, 26.72, 25.71, 24.88, 22.38, 21.63, 20.19, 15.65. HRMS (ESI) m/z calcd for C₂₀H₃₆N₂O₅ ([M+Na]⁺) 407.2509. found 407.2522.

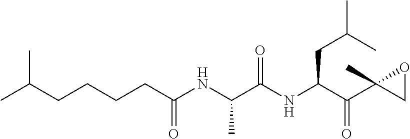 [SMILES: CC(C)CCCCC(=O)N[C@@H](C)C(=O)N[C@@H](CC(C)C)C(=O)[C@@]1(C)CO1]

6-Methyl-N-[(1S)-1-{[(2S)-4-methyl-1-[(2R)-2-methyloxiran-2-yl]-1-oxopentan-2-yl]carbamoyl}ethyl]heptanamide (12j). 2.6 mg (56%), white powder. ¹H NMR (500 MHz, CD₃OD) δ 4.58-4.47 (m, 1H), 4.36 (q, J=7.1 Hz, 1H), 3.27 (d, J=5.2 Hz, 1H), 2.95 (d, J=5.2 Hz, 1H), 2.23 (t, J=7.5 Hz, 2H), 1.76 (ddt, J=13.7, 6.8, 4.0 Hz, 1H), 1.64-1.42 (m, 6H), 1.40-1.27 (m, 7H), 1.22 (dt, J=10.5, 6.8 Hz, 2H), 1.00-0.82 (m, 12H). ¹³C NMR (126 MHz, CD₃OD) δ 208.27, 174.65, 173.90, 58.72, 51.68, 50.48, 48.52, 38.78, 38.45, 35.35, 27.63, 26.68, 25.73, 24.89, 22.35, 21.58, 20.07, 16.59, 15.62. HRMS (ESI) m/z calcd for C₂₀H₃₆N₂O₄ ([M+Na]⁺) 391.2581. found 391.2573.

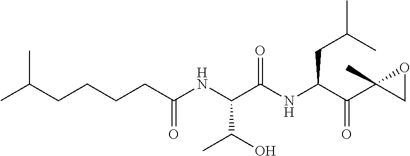 [SMILES: CC(C)CCCCC(=O)N[C@H](C(=O)N[C@@H](CC(C)C)C(=O)[C@@]1(C)CO1)C(C)O]

N-[(1S)-2-Hydroxy-1-{[(2S)-4-methyl-1-[(2R)-2-methyloxiran-2-yl]-1-oxopentan-2-yl]carbamoyl}propyl]-6-methylheptanamide (121). 1.1 mg (24%), white powder. ¹H NMR (500 MHz, CD₃OD) δ 4.58 (dd, J=10.6, 3.1 Hz, 1H), 4.32 (d, J=4.8 Hz, 1H), 4.06 (dd, J=6.6, 5.0 Hz, 1H), 3.27 (d, J=5.0 Hz, 1H), 2.95 (dd, J=5.2, 1.5 Hz, 1H), 2.31 (t, J=7.5 Hz, 2H), 1.75 (ddd, J=10.2, 6.8, 4.0 Hz, 1H), 1.66-1.46 (m, 6H), 1.42-1.30 (m, 3H), 1.27-1.17 (m, 5H), 0.96 (dd, J=12.4, 6.6 Hz, 6H), 0.90 (dd, J=6.6, 1.3 Hz, 6H). ¹³C NMR (126 MHz, CD₃OD) δ 208.27, 175.13, 171.41, 67.09, 58.71, 58.47, 51.71, 50.49, 38.95, 38.48, 35.55, 27.66, 26.69, 25.80, 24.84, 22.35, 21.60, 20.13, 18.75, 15.61. HRMS (ESI) m/z calcd for C₂₁H₃₈N₂O₅ ([M+Na]⁺) 421.2664. found 421.2678.

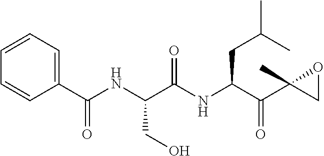 [SMILES: CC(C)C[C@H](NC(=O)[C@H](CO)NC(=O)c1ccccc1)C(=O)[C@@]1(C)CO1]

(2S)-3-Hydroxy-N-[(2S)-4-methyl-1-[(2R)-2-methyloxiran-2-yl]-1-oxopentan-2-yl]-2-(phenylformamido)propenamide (120). 0.6 mg (13%), white powder. ¹H NMR (500 MHz, CDCl₃) δ 7.86-7.77 (m, 2H), 7.56 (t, J=7.3 Hz, 1H), 7.48 (t, J=7.6 Hz, 2H), 7.22 (d, J=7.1 Hz, 1H), 6.96 (d, J=7.7 Hz, 1H), 4.74-4.67 (m, 1H), 4.57 (td, J=8.9, 7.3, 2.9 Hz, 1H), 4.20 (dd, J=11.5, 3.5 Hz, 1H), 3.72 (dd, J=11.5, 5.8 Hz, 1H), 3.33 (d, J=5.0 Hz, 1H), 2.95 (d, J=5.0 Hz, 1H), 1.75-1.59 (m, 2H), 1.56 (d, J=3.6 Hz, 3H), 1.40-1.30 (m, 1H), 0.95 (dd, J=6.4, 4.9 Hz, 6H). ¹³C NMR (126 MHz, CDCl₃) δ 218.54, 208.75, 187.09, 128.73, 127.11, 39.25, 25.37, 21.08, 16.83. HRMS (ESI) m/z calcd for C₁₉H₂₆N₂O₅ ([M+Na]⁺) 385.1729. found 385.1739.

### EpnF Expression and Purification.

A pET-30a expression vector containing EpnF with a C-terminal His₆ tag was purchased from Genscript. E. coli BL21 cells were transformed with the plasmid and cultured in 30 mL LB media at 37° C. overnight. The culture was added to 3 L of Luria-Bertani (LB) media and cultured at 37° C. to OD 0.6. Protein expression was induced by addition of IPTG (0.5 mM final concentration) and the culture was shaken overnight at 18° C. The bacteria were collected by centrifugation (5,000 g) and the pellet lysed by sonication in EpnF Buffer on ice. The protein was purified using HisPur Ni-NTA resin followed by size-exclusion chromatography (GE Life Sciences preparative scale S200 10/300: GE Healthcare, AKTA FPLC equipped with P-920 pump and UPC-900 monitor). The purified EpnF was quantified (6.9 mg/mL, Bradford assay), aliquoted (25 μL), flash frozen on dry ice, and stored at −80° C.

### Example 2. Cytotoxicity Experiments

### Cytotoxicity of Decarboxylation Byproduct 22a.

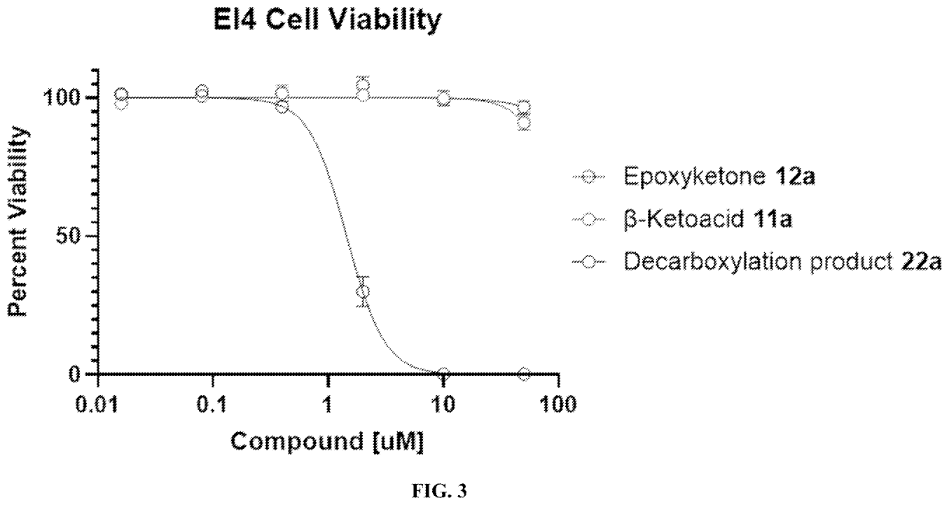

EL4 (mouse lymphoma) cells were cultured in a 96-well plate as described in the Methods section of the manuscript. Epoxyketone 12a, β-ketoacid 11a, and decarboxylation byproduct 22a were dissolved in DMSO (20 mM, 20 μL stock aliquot), then diluted in RPMI media to a concentration of 500 μM (5% DMSO, 75 μL). Compounds were then log-diluted across 10 concentrations (500-0.025 μM) with 5% DMSO in RMPI. Compound dilutions (10 μL) were added to the cells for a final DMSO concentration of 0.5% across all wells. Negative control wells were treated with 5% DMSO in RPMI (10 μL). Cells were incubated at 37° C. (5% CO₂, 95% relative humidity) with compounds for 48 h, then cell viability was determined by CellTitre-Glo assay (Promega G9241) according to the manufacturer's instructions (FIG. 3).

### Cytotoxicity of Purified EpnF Enzyme.

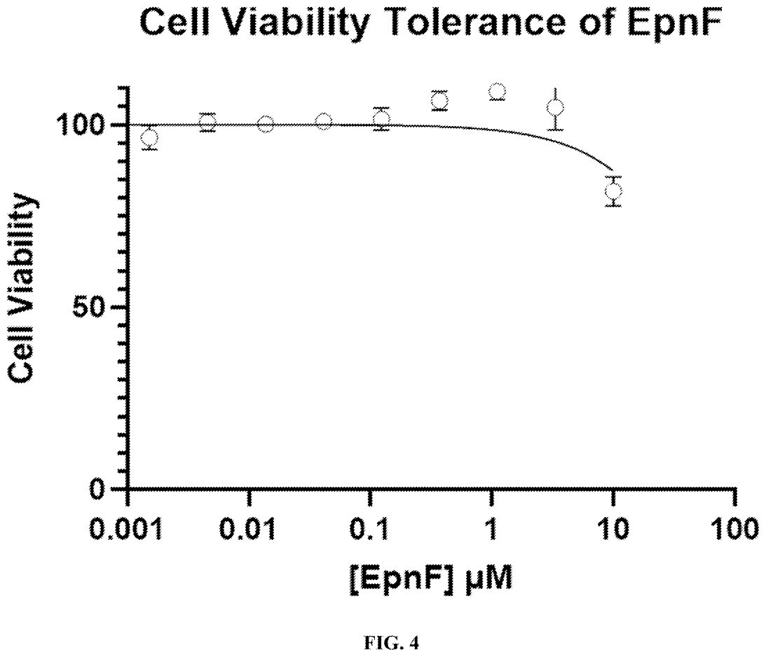

EL4 (mouse lymphoma) cells were cultured in a 96-well plate as described in the Methods section of the manuscript. Recombinant, purified EpnF (100 μM in EpnF Buffer, 75 μL total volume) was diluted across 9 concentrations (10-0.0025 μM) in RPMI (50 μL total volume). Enzyme dilutions (10 μL) were added to the cells. EpnF Buffer was diluted in RPMI as above to serve as a negative control. Negative control wells were treated with EpnF Buffer dilutions (10 μL), across a dilution curve of 10-0.025% EpnF Buffer in RPMI. Cells were incubated at 37° C. (5% CO₂, 95% relative humidity) for 48 h, then cell viability was measured by CellTitre-Glo assay (Promega G9241) according to the manufacturer's instructions (FIG. 4).

### Cytotoxicity of Epoxyketones.

Cytotoxicity Assays. Cancer cell lines (ATCC: U266 multiple myeloma, EL4 mouse lymphoma, Raji B-cell lymphoma) were cultured in Roswell Park Memorial Institute (RPMI) medium supplemented with 10% fetal bovine serum (FBS), 2 mM L-glutamine, 100 IU/mL penicillin, 100 μg/mL nonessential amino acids, 1 mM sodium pyruvate, and 57 μM β-mercaptoethanol. Cells (90 μL) were seeded in the inner 60 wells of an opaque-wall, clear flat-bottom 96-well plate (Corning/Falcon, 351177) at 20,000 cells per well. The outer perimeter wells were filled with 200 μL PBS to reduce evaporation in the central 60 wells. Epoxyketones 12 (20 mM in DMSO) were diluted in RPMI media to 1 mM (5% DMSO final concentration). Each epoxyketone was then diluted log-fold into 10 concentrations with 5% DMSO in RPMI. Each epoxyketone solution (10 μL) was added to the cells for a 0.5% DMSO final concentration across all wells and final epoxyketone concentrations of 100-0.005 μM. Negative control wells were treated with 10 μL of 5% DMSO in RPMI. Cells were incubated with compounds for 48 h, then cytotoxicity was measured with the CellTitre-Glo assay (Promega G9241).

For in situ activation of β-ketoacid 11a by EpnF, U266 cells were seeded in a 96-well plate as above, at 20,000 cells per well in 80 μM RPMI. Recombinant EpnF (100 μM in EpnF Buffer) was diluted in RPMI to 5 μM concentration (300 μL final volume), then 10 μL of this enzyme solution was added to the plate wells to 0.5 μM EpnF final concentration. Stock solutions (20 mM) of β-ketoacid 11a and epoxyketone 12a in EtOH or DMSO, respectively, were diluted to 5% EtOH or DMSO in RPMI to provide working stocks at 1 mM. The working stocks were log-diluted to 1 mM-0.05 μM using RPMI containing 5% EtOH or 5% DMSO, respectively. Aliquots of compounds (10 μL) were added to the sample wells for final concentrations of 100-0.005 μM. Separate negative control wells were treated with 10 μL of 5% EtOH or 5% DMSO in RPMI (0.5% final concentration). The cells were incubated for 48 hr, then cell viability was measured with the CellTitre-Glo assay. Results of compound-treated wells were normalized to negative control wells.

### Proteasome Inhibition Assay.

Epoxyketones 12 were analyzed for proteasome inhibition using a Proteasome Activity Assay Kit (abcam ab107921). The protocol was followed as described by the manufacturer, except that the proteasome sample supplied in the kit was diluted by 10-fold more than indicated in the manual to increase the number of samples that could be analyzed per kit. Thus, the supplied proteasome sample was resuspended in 100 μL ddH₂O, then further diluted with 900 μL of the supplied assay buffer. The supplied proteasome inhibitor, MG-132 (benzyl [(2S)-4-methyl-1-{[(2S)-4-methyl-1-{[(2S)-4-methyl-1-oxopentan-2-yl]amino}-1-oxopentan-2-yl]amino}-1-oxopentan-2-yl]carbamate; structure illustrated below), was also diluted 10-fold to a working concentration of 1 mM in DMSO.

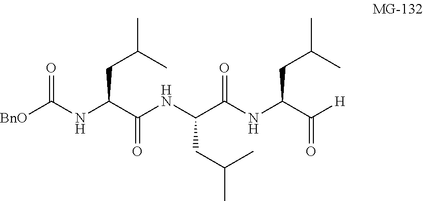 [SMILES: CC(C)C[C@@H](C=O)NC(=O)[C@H](CC(C)C)NC(=O)[C@H](CC(C)C)NC(=O)OCc1ccccc1]

The epoxyketones 12 and epoxomicin positive control (Selleck Chemicals) were also diluted to working stocks of 1 mM in DMSO. Proteasome samples (99 μL) were added to a white, opaque 96-well plate (PerkinElmer, OptiPlate-96) and treated with 1 μL of compound solutions (10 μM final concentration). Samples were incubated for 1 h at 37° C. then analyzed for fluorescence (λ^{ex} 350 nm, λ^{em} 440 nm) on a plate reader (Molecular Devices Spectramax M3). Three separate assay kits were used as biological replicates. Data were normalized to the average signal from the no-inhibitor negative control.

### Discussion and Results

We evaluated the cytotoxicity of the epoxyketones against U266 multiple multiple myeloma, EL4 mouse lymphoma, and Raji B-cell lymphoma cell lines, the results of which are provided in Table 3 below. Epoxyketone 12a has been prepared previously by total synthesis and reported to have an IC₅₀ of 260±50 nM against EL4 lymphoma cells [Kim, K. B.; Myung, J.; Sin, N.; Crews, C. M. (1999) Proteasome inhibition by the natural products epoxomicin and dihydroeponemycin: insights into specificity and potency. Bioorg. Med. Chem. Lett. 9, 3335-3340]. All of the chemoenzymatically produced epoxyketones exhibited cytotoxicity against all three cell lines, at potencies within approximately 1 log of the parent epoxyketone.

TABLE 3

| | U266 | | EL4 | | Raji | |
|---|---|---|---|---|---|---|
| | IC₅₀ | log IC₅₀ | IC₅₀ | log IC₅₀ | IC₅₀ | log IC₅₀ |
| Compound | (μM) | (log μM) | (μM) | (log μM) | (μM) | (log μM) |
| 12a | 0.23 | −0.64 ± | 0.44 | −0.39 ± | 0.20 | −0.71 ± |
| | | 0.07 | | 0.18 | | 0.12 |
| 12j | 2.2 | 0.34 ± | 3.2 | 0.48 ± | 1.9 | 0.28 ± |
| | | 0.04 | | 0.15 | | 0.08 |
| 12l | 2.1 | 0.33 ± | 3.7 | 0.55 ± | 2.1 | 0.32 ± |
| | | 0.02 | | 0.09 | | 0.05 |
| 12n | 4.8 | 0.68 ± | 5.7 | 0.73 ± | 5.4 | 0.73 ± |
| | | 0.03 | | 0.14 | | 0.02 |
| 12o | 2.7 | 0.43 ± | 7.2 | 0.86 ± | 3.6 | 0.54 ± |
| | | 0.06 | | 0.03 | | 0.08 |

Data represent mean ± standard deviation of n = 3 biological replicates. Epoxyketone 12a has been reported to have IC₅₀ = 260 ± 50 nM against EL4 cells.

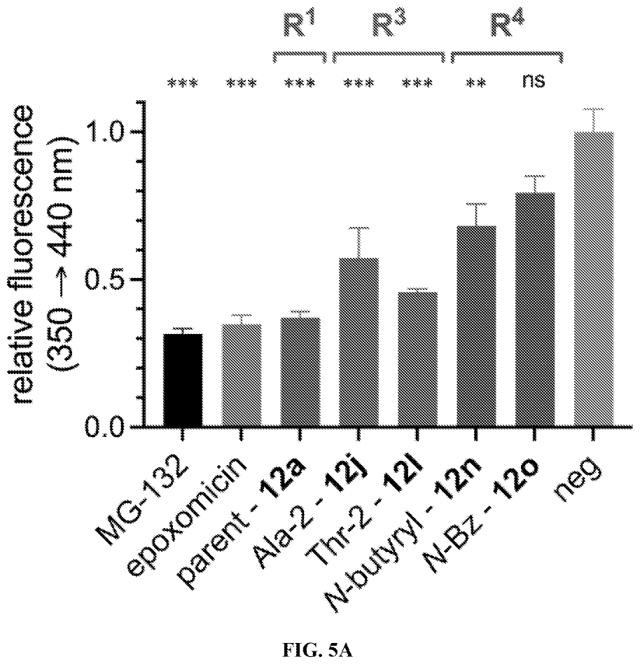

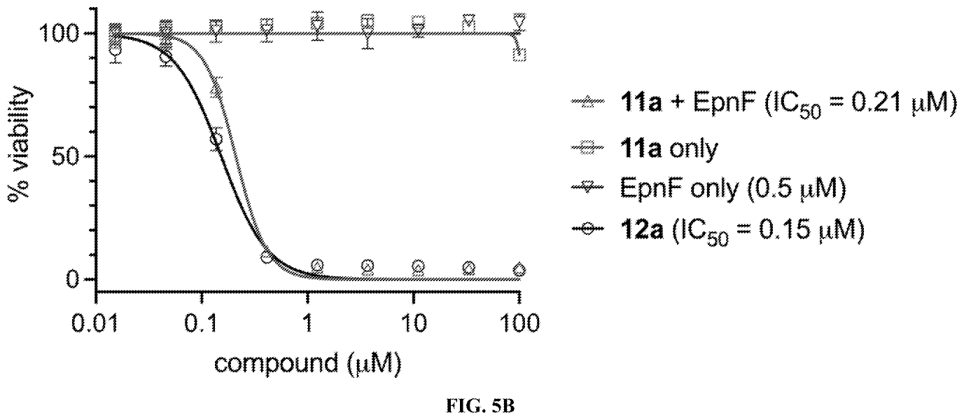

To test whether or not these cytotoxic eponemycin analogues were also proteasome inhibitors, we tested them in an in vitro assay using Jurkat cell lysates having proteasome activity and a fluorescent substrate that is cleaved by the chymotrypsin-like activity of the proteasome (abcam, Proteasome Activity Assay Kit). At 10 μM concentration, the parent epoxyketone 12a inhibited proteasome activity at a level comparable to that of epoxomicin (Selleck Chemicals) and a known proteasome inhibitor MG-132 (FIG. 5A). The four eponemycin analogues 12j, 12l, 12n, and 12o also inhibited proteasome activity by 21-55% under these conditions. Rank ordering generally mirrored that seen in the cell cytotoxicity assay, consistent with proteasome inhibition as a mechanism of action.

Finally, to demonstrate the potential utility of these β-ketoacids in enzyme-prodrug systems, we evaluated their in situ activation by EpnF. U266 cells were treated with β-ketoacid 11a, in the absence or presence of recombinant, purified EpnF (FIG. 5B). Strikingly, the combination of β-ketoacid 11a and EpnF (0.5 μM) exhibited cytotoxicity comparable to that of the corresponding isolated epoxyketone 12a. The prodrug alone exhibited no cytotoxicity at up to 100 μM concentration. In separate experiments, we also confirmed that the corresponding decarboxylation byproduct 22a was non-cytotoxic at up to 50 μM (see FIG. 3), as was recombinant, purified EpnF at up to 3 μM (see FIG. 4).

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such
A. An epoxyketone compound of Formula (I)

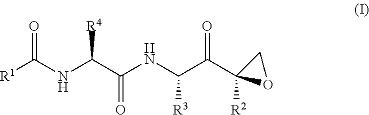

or a pharmaceutically acceptable salt and/or solvate thereof, wherein
   R¹ is alkyl, unsubstituted phenyl, or substituted phenyl;
   R² is H or C₁₋₆ alkyl optionally substituted with OH;
   R³ is C₁₋₆ alkyl optionally substituted with C₂₋₆ alkenyl or phenyl;
   R⁴ is C₁₋₆ alkyl optionally substituted with OH or phenyl; and
wherein the epoxyketone compound is not

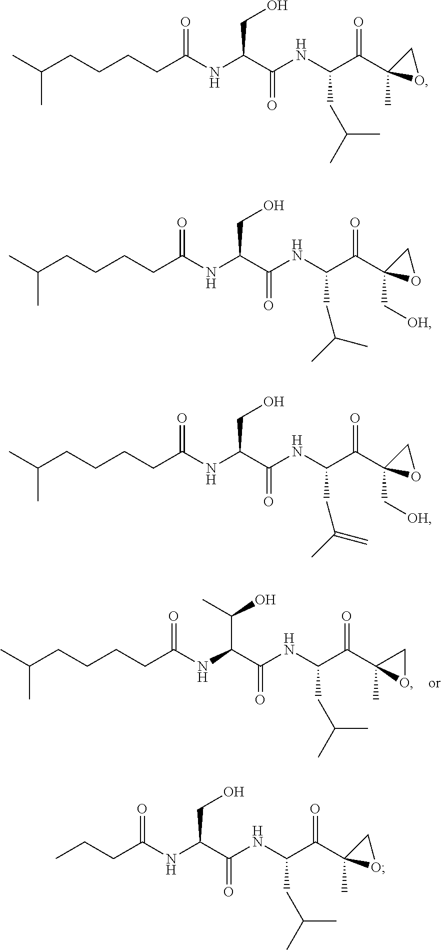 [SMILES: CC(C)CCCCC(=O)N[C@@H](CO)C(=O)N[C@@H](CC(C)C)C(=O)[C@@]1(C)CO1 ; CC(C)CCCCC(=O)N[C@@H](CO)C(=O)N[C@@H](CC(C)C)C(=O)[C@@]1(CO)CO1 ; C=C(C)C[C@H](NC(=O)[C@H](CO)NC(=O)CCCCC(C)C)C(=O)[C@@]1(CO)CO1 ; CC(C)CCCCC(=O)N[C@H](C(=O)N[C@@H](CC(C)C)C(=O)[C@@]1(C)CO1)[C@@H](C)O ; CCCC(=O)N[C@@H](CO)C(=O)N[C@@H](CC(C)C)C(=O)[C@@]1(C)CO1]

and
optionally wherein the epoxyketone compound is not

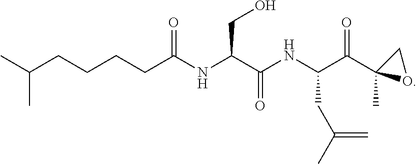 [SMILES: C=C(C)C[C@H](NC(=O)[C@H](CO)NC(=O)CCCCC(C)C)C(=O)[C@@]1(C)CO1]

B. The epoxyketone compound of Paragraph A, wherein the epoxyketone compound is according to Formula (II)

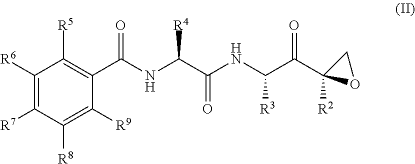

or a pharmaceutically acceptable salt and/or solvate thereof, wherein
   R⁵, R⁶, R⁷, R⁸, and R⁹ are each independently selected from —H, —F, —Cl, —Br, —I, alkoxy, —OH, —CF₃, —CN, —COOH, —COOR¹⁰, —C(O)NR¹¹R¹², —CHNOH, —NR¹³R¹⁴, —NHNH₂, —NO₂, —N₃, —NR¹⁵C(O)R¹⁶, —NR¹⁷C(O)OR¹⁸, —OC(O)NR¹⁹R²⁰, —SH, —SR²¹, —S(O)R²², —SO₂R²³, —SO₂OR²⁴, alkyl, heteroalkyl, alkenyl, alkynyl, heterocycloalkyl, aryl, and heteroaryl.
C. The epoxyketone compound of Paragraph B, wherein R⁵, R⁶, R⁷, R⁸, and R⁹ are each independently —H.
D. The epoxyketone compound of Paragraph A, wherein R¹ is alkyl.
E. The epoxyketone compound of Paragraph A or Paragraph D, wherein
   R¹ is unsubstituted C₁₋₁₈ alkyl.
F. The epoxyketone compound of any one of Paragraphs A-E, wherein R⁴ is unsubstituted C₁₋₆ alkyl or C₁₋₆ alkyl substituted with a phenyl group.
G. The epoxyketone compound of any one of Paragraphs A and D-F, wherein the epoxyketone compound is according to Formula (III)

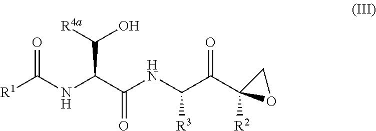

or a pharmaceutically acceptable salt and/or solvate thereof, wherein
   R¹ is unsubstituted C₁₋₁₈ alkyl; and
   R^{4a} is H or C₁₋₆ alkyl.
H. The epoxyketone compound of any one of Paragraphs A-G, wherein when R² is unsubstituted C₁₋₆ alkyl, R³ is not unsubstituted C₁₋₆ alkyl.
I. The epoxyketone compound of any one of Paragraphs A-H, wherein when R² is hydroxyl substituted C₁₋₆ alkyl, R³ is not unsubstituted C₁₋₆ alkyl or C₁₋₆ alkyl substituted with a C₂₋₆ alkenyl group.
J. The epoxyketone compound of any one of Paragraphs A-I, wherein R² is H, CH₃, or CH₂OH.
K. The epoxyketone compound of any one of Paragraphs A-J, wherein R³ is

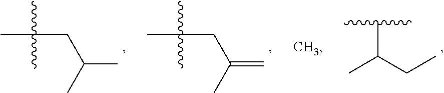

CH(CH₃)₂, or benzyl.
L. The epoxyketone compound of any one of Paragraphs A-K, wherein R⁴ is CH₂OH, benzyl, CH(OH)CH₃, CH(CH₃)₂, or CH₃.
M. The epoxyketone compound of any one of Paragraphs A-L, wherein the epoxyketone compound is

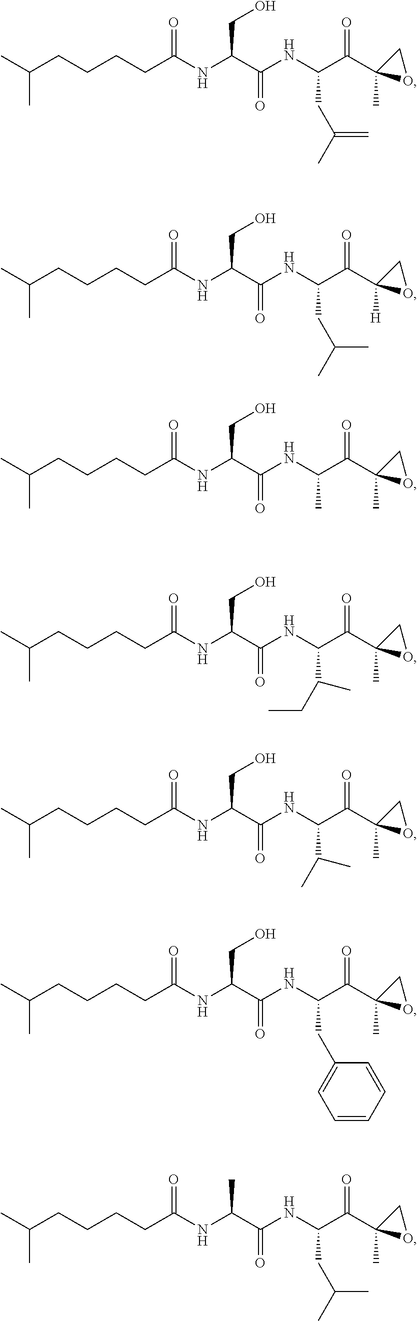

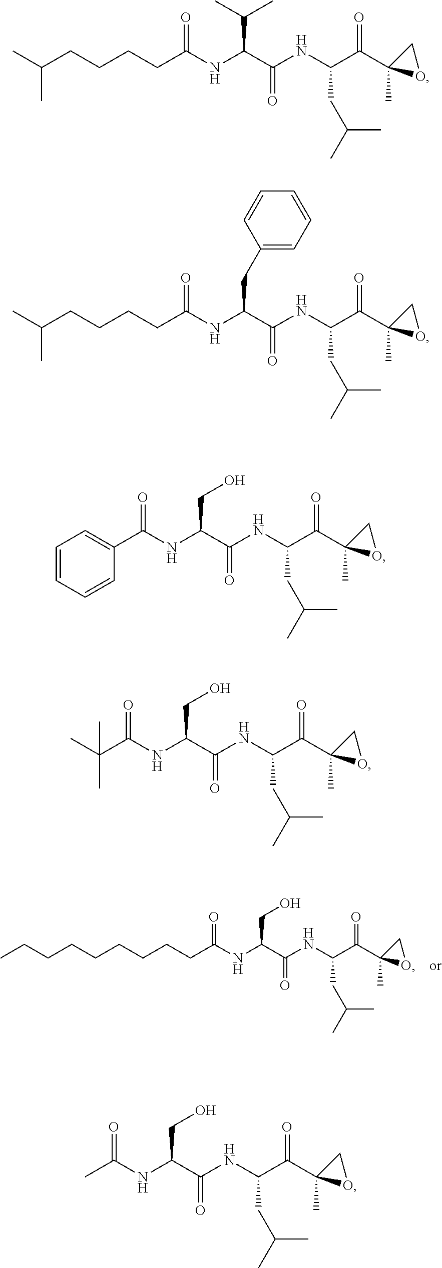 [SMILES: CC(C)CCCCC(=O)N[C@H](C(=O)N[C@@H](CC(C)C)C(=O)[C@@]1(C)CO1)C(C)C ; CC(C)CCCCC(=O)N[C@@H](Cc1ccccc1)C(=O)N[C@@H](CC(C)C)C(=O)[C@@]1(C)CO1 ; CC(C)C[C@H](NC(=O)[C@H](CO)NC(=O)c1ccccc1)C(=O)[C@@]1(C)CO1 ; CC(C)C[C@H](NC(=O)[C@H](CO)NC(=O)C(C)(C)C)C(=O)[C@@]1(C)CO1 ; CCCCCCCCCC(=O)N[C@@H](CO)C(=O)N[C@@H](CC(C)C)C(=O)[C@@]1(C)CO1 ; CC(=O)N[C@@H](CO)C(=O)N[C@@H](CC(C)C)C(=O)[C@@]1(C)CO1]

or a pharmaceutically acceptable salt and/or solvate thereof.
N. The epoxyketone compound of any one of Paragraphs A-MK wherein the epoxyketone compound is

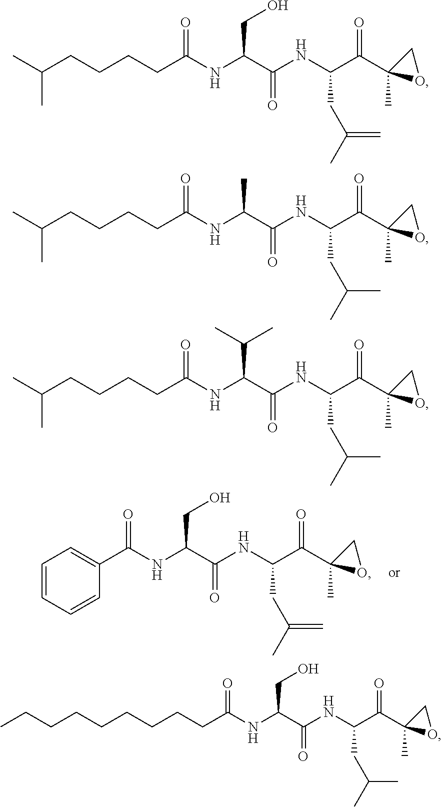

or a pharmaceutically acceptable salt and/or solvate thereof.
O. A composition comprising
   an epoxyketone compound of any one of Paragraphs A-N; and
   a pharmaceutically acceptable carrier.
P. A pharmaceutical composition comprising
   an effective amount of an epoxyketone compound of any one of Paragraphs A-N to treat a disease associated with proteasome activity; and
   a pharmaceutically acceptable carrier.
Q. The pharmaceutical composition of Paragraph P, wherein the disease is cancer.
R. The pharmaceutical composition of Paragraph P or Paragraph Q, wherein the disease is a hematopoietic cancer.
S. The pharmaceutical composition of Paragraph P or Paragraph Q, wherein the disease is acute lymphoblastic leukemia (ALL).
T. The pharmaceutical composition of Paragraph P or Paragraph Q, wherein the disease is multiple myeloma.
U. A method of treating a disease comprising administering to a subject an effective amount of an epoxyketone compound of any one of Paragraphs A-N.
V. The method of Paragraph U, wherein the disease is associated with proteasome activity.
W. The method of Paragraph U or Paragraph V, wherein the disease is cancer.
X. The method of Paragraph W, wherein the cancer is a hematopoietic cancer.
Y. The method of Paragraph W, wherein the cancer is acute lymphoblastic leukemia (ALL).
Z. The method of Paragraph W, wherein the cancer is multiple myeloma.
AA. A β-ketoacid of Formula (A)

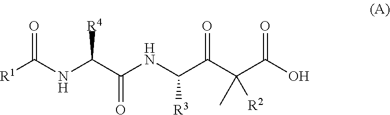

or a pharmaceutically acceptable salt and/or solvate thereof, wherein
   R¹ is alkyl, unsubstituted phenyl, or substituted phenyl;
   R² is H or C₁₋₆ alkyl optionally substituted with OH;
   R³ is C₁₋₆ alkyl optionally substituted with C₂₋₆ alkenyl or phenyl;
   R⁴ is C₁₋₆ alkyl optionally substituted with OH or phenyl; and
wherein the β-ketoacid is not

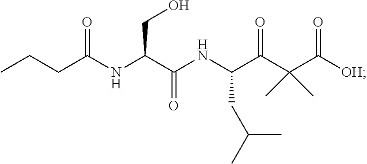 [SMILES: CCCC(=O)N[C@@H](CO)C(=O)N[C@@H](CC(C)C)C(=O)C(C)(C)C(=O)O]

optionally wherein the β-ketoacid is not one or more of

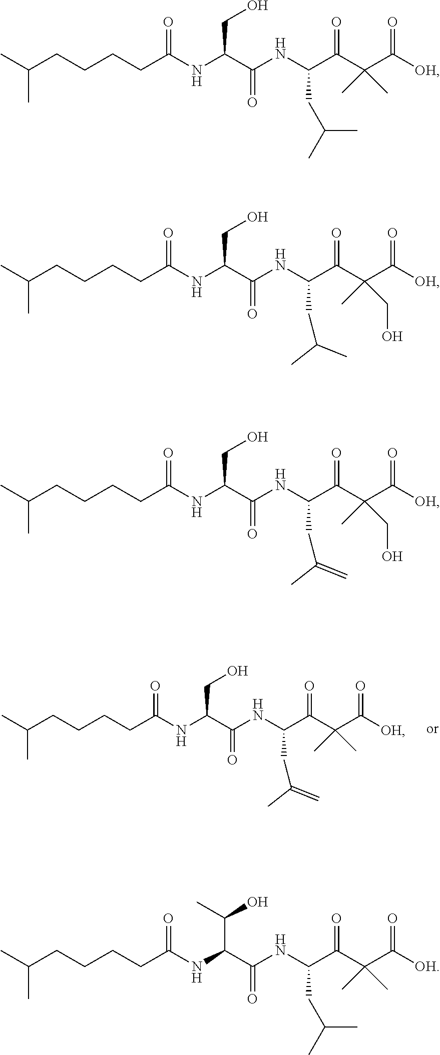 [SMILES: CC(C)CCCCC(=O)N[C@@H](CO)C(=O)N[C@@H](CC(C)C)C(=O)C(C)(C)C(=O)O ; CC(C)CCCCC(=O)N[C@@H](CO)C(=O)N[C@@H](CC(C)C)C(=O)C(C)(CO)C(=O)O ; C=C(C)C[C@H](NC(=O)[C@H](CO)NC(=O)CCCCC(C)C)C(=O)C(C)(CO)C(=O)O ; C=C(C)C[C@H](NC(=O)[C@H](CO)NC(=O)CCCCC(C)C)C(=O)C(C)(C)C(=O)O ; CC(C)CCCCC(=O)N[C@H](C(=O)N[C@@H](CC(C)C)C(=O)C(C)(C)C(=O)O)[C@@H](C)O]

AB. The β-ketoacid of Paragraph AA, wherein the β-ketoacid is according to Formula (B)

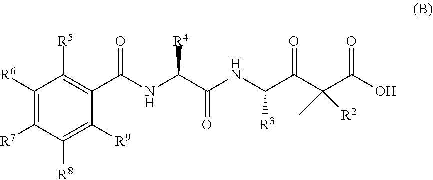

or a pharmaceutically acceptable salt and/or solvate thereof, wherein
   R⁵, R⁶, R⁷, R⁸, and R⁹ are each independently selected from —H, —F, —Cl, —Br, —I, alkoxy, —OH, —CF₃, —CN, —COOH, —COOR¹⁰, —C(O)NR¹¹R¹², —CHNOH, —NR¹³R¹⁴, —NHNH₂, —NO₂, —N₃, —NR¹⁵C(O)R¹⁶, —NR¹⁷C(O)OR¹⁸, —OC(O)NR¹⁹R²⁰, —SH, —SR²¹, —S(O)R²², —SO₂R²³, —SO₂OR²⁴, alkyl, heteroalkyl, alkenyl, alkynyl, heterocycloalkyl, aryl, and heteroaryl.
AC. The β-ketoacid of Paragraph AB, wherein R⁵, R⁶, R⁷, R⁸, and R⁹ are each independently
   —H.
AD. The β-ketoacid of Paragraph AA, wherein R¹ is alkyl.
AE. The β-ketoacid of Paragraph AA or Paragraph AD, wherein R¹ is unsubstituted C₁₋₁₈ alkyl.
AF. The β-ketoacid of Paragraph AE, wherein R⁴ is unsubstituted C₁₋₆ alkyl or C₁₋₆ alkyl substituted with a phenyl group.
AG. The β-ketoacid of any one of Paragraphs AA and AD-AF, having a structure according to Formula (C)

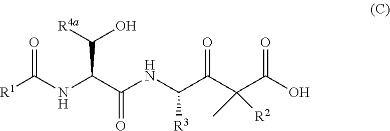

or a pharmaceutically acceptable salt and/or solvate thereof, wherein
   R¹ is unsubstituted C₁₋₁₈ alkyl; and
   R^{4a} is H or C₁₋₆ alkyl.
AH. The β-ketoacid of any one of Paragraphs AA-AG, wherein when R² is unsubstituted C₁₋₆ alkyl, R³ is not unsubstituted C₁₋₆ alkyl.
AI. The β-ketoacid of any one of Paragraphs AA-AH, wherein when R² is hydroxyl substituted C₁₋₆ alkyl, R³ is not unsubstituted C₁₋₆ alkyl or C₁₋₆ alkyl substituted with a C₂₋₆ alkenyl group.
AJ. The β-ketoacid of any one of Paragraphs AA-AI, wherein R² is H, CH₃, or CH₂OH.
AK. The β-ketoacid of an one of Paragraphs AA-AJ, wherein R³ is

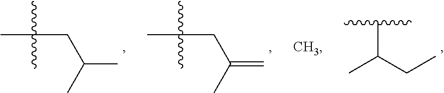

CH(CH₃)₂, or benzyl.
AL. The β-ketoacid of any one of Paragraphs AA-AK, wherein R⁴ is CH₂OH, benzyl, CH(OH)CH₃, CH(CH₃)₂, or CH₃.
AM. The β-ketoacid of any one of Paragraphs AA-AL, wherein the β-ketoacid is

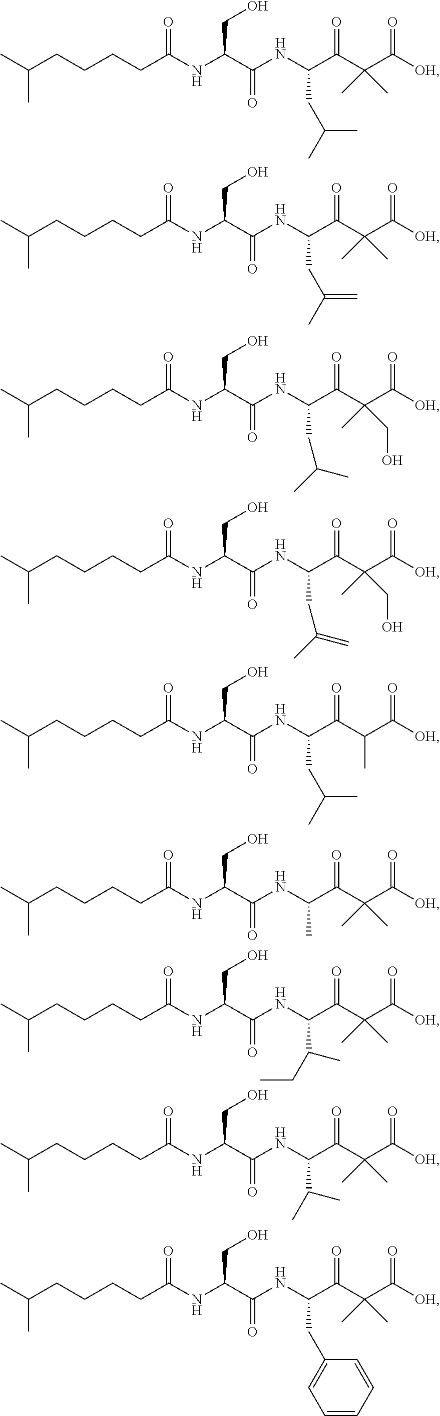

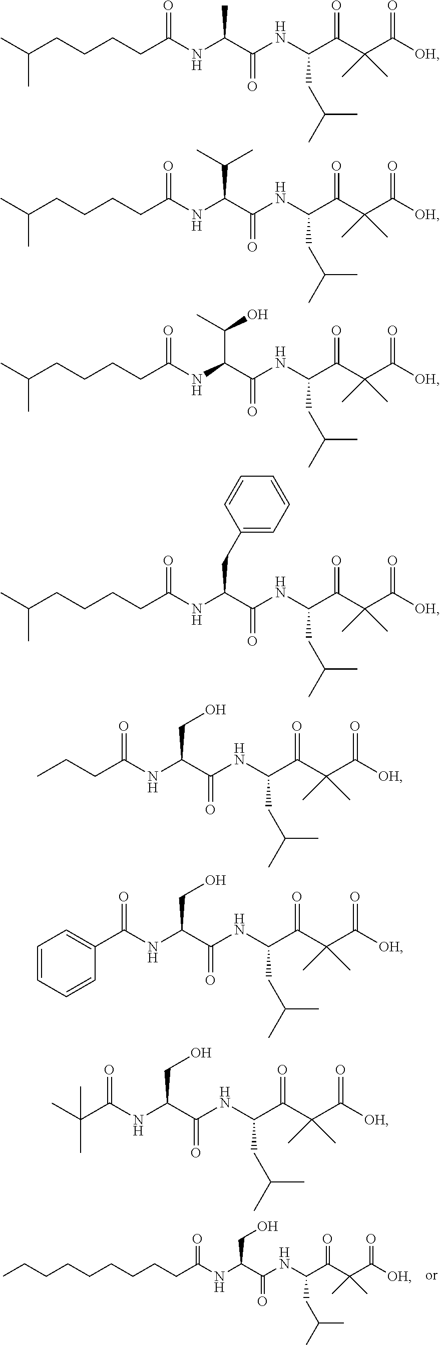

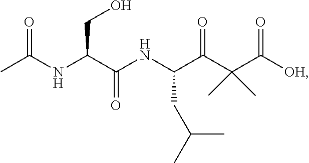 [SMILES: CC(=O)N[C@@H](CO)C(=O)N[C@@H](CC(C)C)C(=O)C(C)(C)C(=O)O]

or a pharmaceutically acceptable salt and/or solvate thereof.
AN. A composition comprising
   a β-ketoacid of any one of Paragraphs AA-AM; and
   a pharmaceutically acceptable carrier.
AO. A pharmaceutical composition comprising
   an effective amount of a β-ketoacid of any one of Paragraphs AA-AM to treat a disease associated with proteasome activity; and
   a pharmaceutically acceptable carrier.
AP. The pharmaceutical composition of Paragraph AO, wherein the disease is cancer.
AQ. The pharmaceutical composition of Paragraph AO or Paragraph AP, wherein the disease is a hematopoietic cancer.
AR. The pharmaceutical composition of Paragraph AO or Paragraph AP, wherein the disease is acute lymphoblastic leukemia (ALL).
AS. The pharmaceutical composition of Paragraph AO or Paragraph AP, wherein the disease is multiple myeloma.
AT. A method of treating a disease comprising administering to a subject an effective amount of a β-ketoacid of any one of Paragraphs AA-AM and administering to the subject an effective amount of an epoxyketone synthase.
AU. The method of Paragraph AT, wherein the disease is associated with proteasome activity.
AV. The method of Paragraph AT or Paragraph AU, wherein the disease is cancer.
AW. The method of Paragraph AV, wherein the cancer is a hematopoietic cancer, acute lymphoblastic leukemia (ALL), and/or multiple myeloma.
AX. The method of Paragraph AV or Paragraph AW, wherein the method comprises administering the epoxyketone synthase locally to a tumor comprising the cancer.
AY. The method of Paragraph AX, wherein the method comprises intratumoral injection of the epoxyketone synthase.
AZ. The method of any one of Paragraphs AT-AY, wherein the epoxyketone synthase is EpnF, EpxF, TmcF, MynC, AR412 ACAD, AR456 ACAD, AB162 ACAD, NM1663 ACAD, AZ379 ACAD, AB9 ACAD, AB1383 ACAD, AB1241 ACAD, or AZ40 ACAD.
BA. The method of any one of Paragraphs AT-AZ, wherein the epoxyketone synthase is EpnF.
BB. The method of any one of Paragraphs AT-BA, wherein the epoxyketone synthase is EpxF.
BC. A process for preparing an epoxyketone compound of any one of Paragraphs A-N, the process comprising contacting a β-ketoacid of any one of Paragraphs AA-AM with an epoxyketone synthase to provide the epoxyketone compound.
BD. The process of Paragraph BC, wherein the contacting occurs at a temperature of about 35° C. to about 40° C.
BE. The process of Paragraph BC or Paragraph BD, wherein the contacting occurs at a pH of about 7.5.
BF. The process of Paragraph BE, wherein the contacting occurs in a Tris (tris(hydroxymethyl)aminomethane) buffer solution.
BG. The process of any one of Paragraphs BC-BF, wherein the epoxyketone synthase is EpnF, EpxF, TmcF, MynC, AR412 ACAD, AR456 ACAD, AB162 ACAD, NM1663 ACAD, AZ379 ACAD, AB9 ACAD, AB1383 ACAD, AB1241 ACAD, or AZ40 ACAD.
BH. The method of any one of Paragraphs BC-BG, wherein the epoxyketone synthase is EpnF.
BI. The method of any one of Paragraphs BC-BG, wherein the epoxyketone synthase is EpxF.
Other embodiments are set forth in the following claims, along with the full scope of equivalents to which such claims are entitled.

1. An epoxyketone compound of Formula (I) 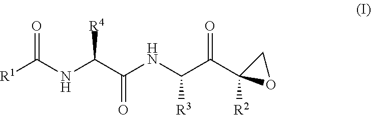
or a pharmaceutically acceptable salt and/or solvate thereof, wherein
R¹ is alkyl, unsubstituted phenyl, or substituted phenyl;
R² is H or C₁₋₆ alkyl optionally substituted with OH;
R³ is C₁₋₆ alkyl optionally substituted with C₂₋₆ alkenyl or phenyl;
R⁴ is C₁₋₆ alkyl optionally substituted with OH or phenyl; and
wherein the epoxyketone compound is not 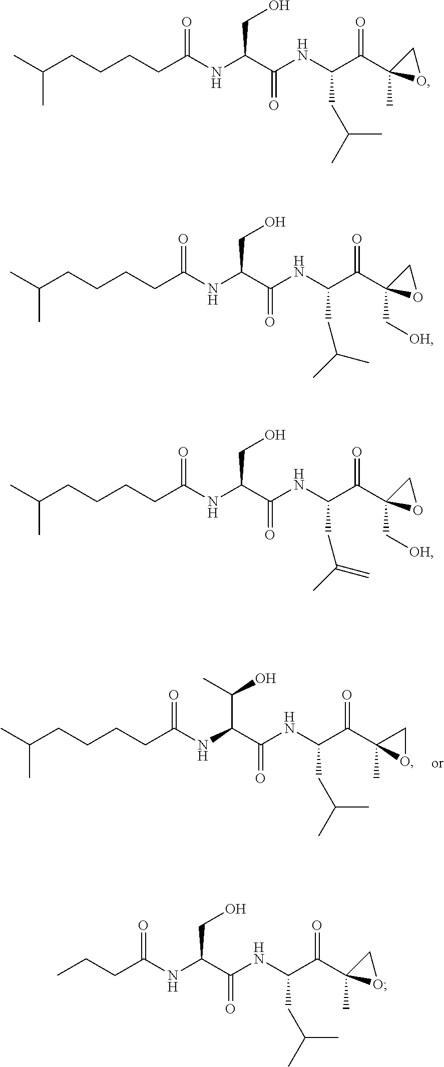 [SMILES: CC(C)CCCCC(=O)N[C@@H](CO)C(=O)N[C@@H](CC(C)C)C(=O)[C@@]1(C)CO1 ; CC(C)CCCCC(=O)N[C@@H](CO)C(=O)N[C@@H](CC(C)C)C(=O)[C@@]1(CO)CO1 ; C=C(C)C[C@H](NC(=O)[C@H](CO)NC(=O)CCCCC(C)C)C(=O)[C@@]1(CO)CO1 ; CC(C)CCCCC(=O)N[C@H](C(=O)N[C@@H](CC(C)C)C(=O)[C@@]1(C)CO1)[C@@H](C)O ; CCCC(=O)N[C@@H](CO)C(=O)N[C@@H](CC(C)C)C(=O)[C@@]1(C)CO1]
and
optionally wherein the epoxyketone compound is not

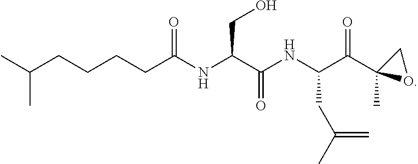 [SMILES: C=C(C)C[C@H](NC(=O)[C@H](CO)NC(=O)CCCCC(C)C)C(=O)[C@@]1(C)CO1]

2. The epoxyketone compound of claim 1, wherein the epoxyketone compound is according to Formula (II) 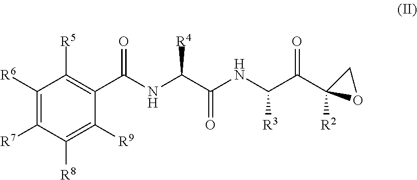
or a pharmaceutically acceptable salt and/or solvate thereof, wherein
R⁵, R⁶, R⁷, R⁸, and R⁹ are each independently selected from —H, —F, —Cl, —Br, —I, alkoxy, —OH, —CF₃, —CN, —COOH, —COOR¹⁰, —C(O)NR¹¹R¹², —CHNOH, —NR¹³R¹⁴, —NHNH₂, —NO₂, —N₃, —NR¹⁵C(O)R¹⁶, —NR¹⁷C(O)OR¹⁸, —OC(O)NR¹⁹R²⁰, —SH, —SR²¹, —S(O)R²², —SO₂R²³, —SO₂OR²⁴, alkyl, heteroalkyl, alkenyl, alkynyl, heterocycloalkyl, aryl, and heteroaryl.

3. The epoxyketone compound of claim 2, wherein R⁵, R⁶, R⁷, R⁸, and R⁹ are each independently —H.

4. (canceled)

5. The epoxyketone compound of claim 1, wherein R¹ is unsubstituted C₁₋₁₈ alkyl.

6. The epoxyketone compound of claim 1, wherein R⁴ is unsubstituted C₁₋₆ alkyl or C₁₋₆ alkyl substituted with a phenyl group.

7. The epoxyketone compound of claim 1, wherein the epoxyketone compound is according to Formula (III) 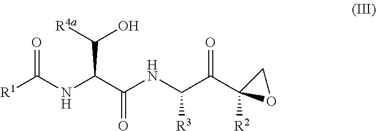
or a pharmaceutically acceptable salt and/or solvate thereof, wherein
R¹ is unsubstituted C₁₋₁₈ alkyl; and
R^{4a} is H or C₁₋₆ alkyl.

8. The epoxyketone compound of claim 1, wherein when R² is unsubstituted C₁₋₆ alkyl, R³ is not unsubstituted C₁₋₆ alkyl.

9. The epoxyketone compound of claim 1, wherein when R² is hydroxyl substituted C₁₋₆ alkyl, R³ is not unsubstituted C₁₋₆ alkyl or C₁₋₆ alkyl substituted with a C₂₋₆ alkenyl group.

10. The epoxyketone compound of claim 1, wherein R² is H, CH₃, or CH₂OH.

11. The epoxyketone compound of claim 1, wherein R³ is 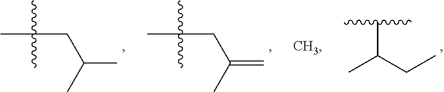
CH(CH₃)₂, or benzyl.

12. The epoxyketone compound of claim 1, wherein R⁴ is CH₂OH, benzyl, CH(OH)CH₃, CH(CH₃)₂, or CH₃.

13. The epoxyketone compound of claim 1, wherein the epoxyketone compound is 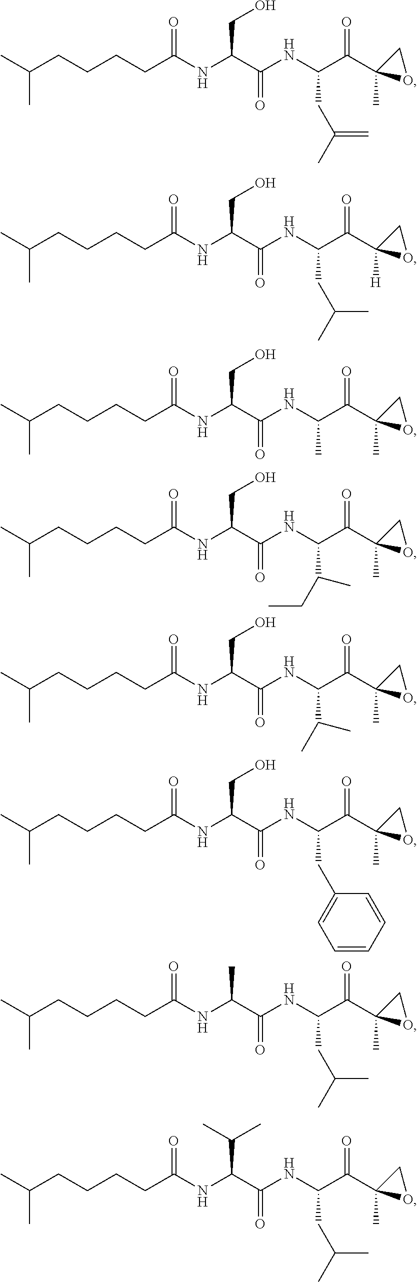 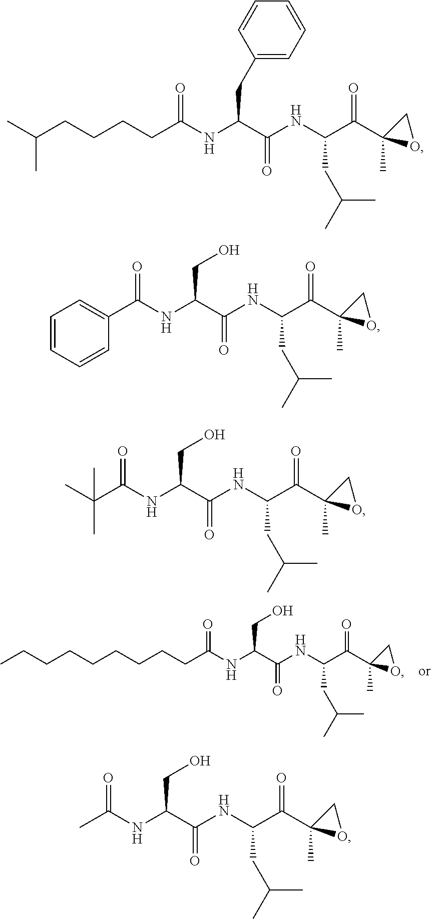
or a pharmaceutically acceptable salt and/or solvate thereof.

14. The epoxyketone compound of claim 1, wherein the epoxyketone compound is 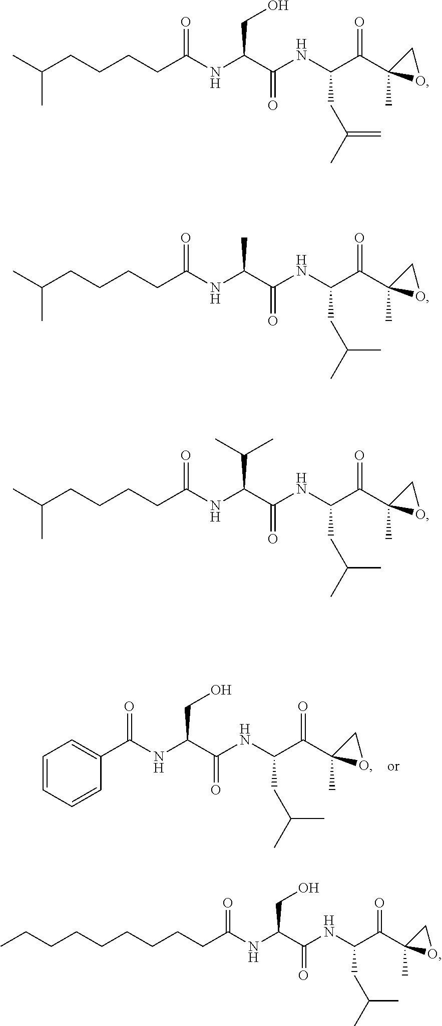 [SMILES: C=C(C)C[C@H](NC(=O)[C@H](CO)NC(=O)CCCCC(C)C)C(=O)[C@@]1(C)CO1 ; CC(C)CCCCC(=O)N[C@@H](C)C(=O)N[C@@H](CC(C)C)C(=O)[C@@]1(C)CO1 ; CC(C)CCCCC(=O)N[C@H](C(=O)N[C@@H](CC(C)C)C(=O)[C@@]1(C)CO1)C(C)C ; CC(C)C[C@H](NC(=O)[C@H](CO)NC(=O)c1ccccc1)C(=O)[C@@]1(C)CO1 ; CCCCCCCCCC(=O)N[C@@H](CO)C(=O)N[C@@H](CC(C)C)C(=O)[C@@]1(C)CO1]
or a pharmaceutically acceptable salt and/or solvate thereof.

15. A composition comprising
an epoxyketone compound of claim 1; and
a pharmaceutically acceptable carrier.

16. A pharmaceutical composition comprising
an effective amount of an epoxyketone compound of claim 1 to treat a disease associated with proteasome activity; and
a pharmaceutically acceptable carrier.

17.-26. (canceled)

27. A β-ketoacid of Formula (A) 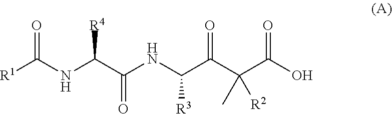
or a pharmaceutically acceptable salt and/or solvate thereof, wherein
R¹ is alkyl, unsubstituted phenyl, or substituted phenyl;
R² is H or C₁₋₆ alkyl optionally substituted with OH;
R³ is C₁₋₆ alkyl optionally substituted with C₂₋₆ alkenyl or phenyl;
R⁴ is C₁₋₆ alkyl optionally substituted with OH or phenyl; and
wherein the β-ketoacid is not 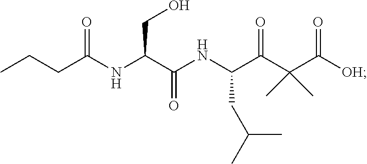 [SMILES: CCCC(=O)N[C@@H](CO)C(=O)N[C@@H](CC(C)C)C(=O)C(C)(C)C(=O)O]
optionally wherein the β-ketoacid is not one or more of

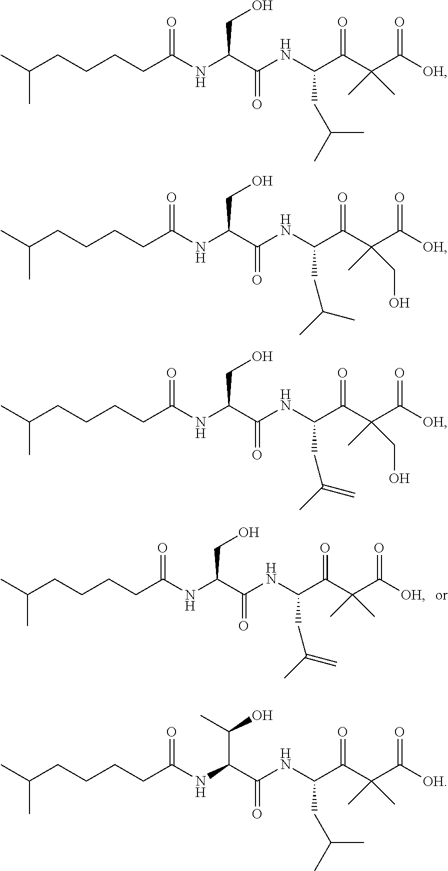

28.-39. (canceled)

40. A composition comprising
a β-ketoacid of claim 27; and
a pharmaceutically acceptable carrier.

41. A pharmaceutical composition comprising
an effective amount of a β-ketoacid of claim 27 to treat a disease associated with proteasome activity; and
a pharmaceutically acceptable carrier.

42.-45. (canceled)

46. A method of treating a disease comprising administering to a subject an effective amount of a β-ketoacid of claim 27 and administering to the subject an effective amount of an epoxyketone synthase.

47.-54. (canceled)

55. A process for preparing an epoxyketone compound of claim 1, the process comprising contacting a β-ketoacid with an epoxyketone synthase to provide the epoxyketone compound; wherein the β-ketoacid is of Formula (A) 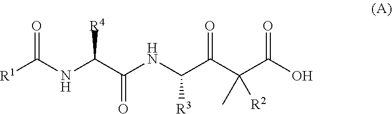
or a pharmaceutically acceptable salt and/or solvate thereof, wherein
R¹ is alkyl, unsubstituted phenyl, or substituted phenyl;
R² is H or C₁₋₆ alkyl optionally substituted with OH;
R³ is C₁₋₆ alkyl optionally substituted with C₂₋₆ alkenyl or phenyl;
R⁴ is C₁₋₆ alkyl optionally substituted with OH or phenyl; and
wherein the β-ketoacid is not 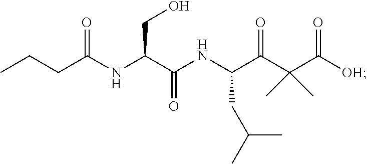 [SMILES: CCCC(=O)N[C@@H](CO)C(=O)N[C@@H](CC(C)C)C(=O)C(C)(C)C(=O)O]
optionally wherein the β-ketoacid is not one or more of

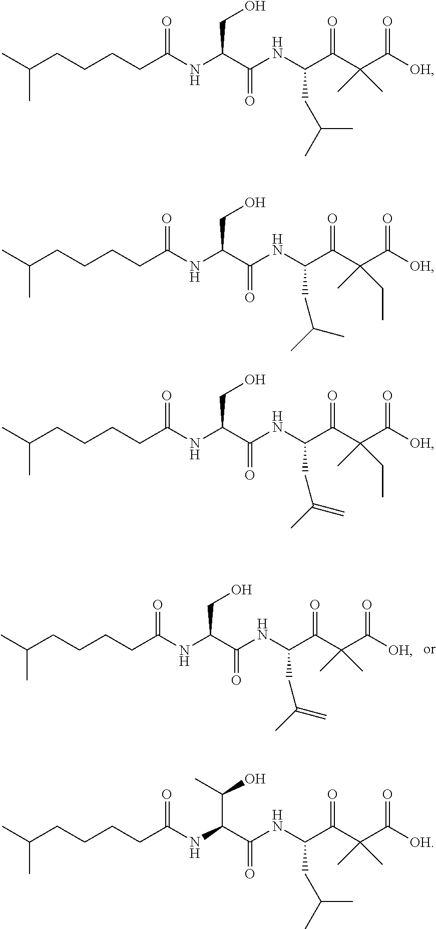 [SMILES: CC(C)CCCCC(=O)N[C@@H](CO)C(=O)N[C@@H](CC(C)C)C(=O)C(C)(C)C(=O)O ; CCC(C)(C(=O)O)C(=O)[C@H](CC(C)C)NC(=O)[C@H](CO)NC(=O)CCCCC(C)C ; C=C(C)C[C@H](NC(=O)[C@H](CO)NC(=O)CCCCC(C)C)C(=O)C(C)(CC)C(=O)O ; C=C(C)C[C@H](NC(=O)[C@H](CO)NC(=O)CCCCC(C)C)C(=O)C(C)(C)C(=O)O ; "or" ; CC(C)CCCCC(=O)N[C@H](C(=O)N[C@@H](CC(C)C)C(=O)C(C)(C)C(=O)O)[C@@H](C)O]

56.-61. (canceled)
